SEQUENCE LISTING (260 sequences)

SEQ ID NO: 1 - protein, 35 aa, Stichodactyla helianthus
RSCIDTIPKSRCTAFQCKHSMKYRLSFCRKTCGTC

SEQ ID NO: 2 - protein, 35 aa, Artificial Sequence
  ShK-Based Peptide
RSCIDTIPKSRCTAFQSKHSMKYRLSFCRKTSGTC

SEQ ID NO: 3 - protein, 35 aa, Artificial Sequence
  ShK-Based Peptide
RSSIDTIPKSRCTAFQCKHSMKYRLSFCRKTCGTS

SEQ ID NO: 4 - protein, 35 aa, Artificial Sequence
  ShK-Based Peptide
SSCIDTIPKSRCTAFQCKHSMKYRLSFCRKTCGTC

SEQ ID NO: 5 - protein, 35 aa, Artificial Sequence
  ShK-Based Peptide
  MISC_FEATURE (1)..(1): N-acetylarginine
XSCIDTIPKSRCTAFQCKHSMKYRLSFCRKTCGTC

SEQ ID NO: 6 - protein, 34 aa, Artificial Sequence
  ShK-Based Peptide
SCIDTIPKSRCTAFQCKHSMKYRLSFCRKTCGTC

SEQ ID NO: 7 - protein, 33 aa, Artificial Sequence
  ShK-Based Peptide
CIDTIPKSRCTAFQCKHSMKYRLSFCRKTCGTC

SEQ ID NO: 8 - protein, 35 aa, Artificial Sequence
  ShK-Based Peptide
ASCIDTIPKSRCTAFQCKHSMKYRLSFCRKTCGTC

SEQ ID NO: 9 - protein, 34 aa, Artificial Sequence
  ShK-Based Peptide
QCIDTIPKSRCTAFQCKHSMKYRLSFCRKTCGTC

SEQ ID NO: 10 - protein, 34 aa, Artificial Sequence
  ShK-Based Peptide
ACIDTIPKSRCTAFQCKHSMKYRLSFCRKTCGTC

SEQ ID NO: 11 - protein, 34 aa, Artificial Sequence
  ShK-Based Peptide
TCIDTIPKSRCTAFQCKHSMKYRLSFCRKTCGTC

SEQ ID NO: 12 - protein, 35 aa, Artificial Sequence
  ShK-Based Peptide
RQCIDTIPKSRCTAFQCKHSMKYRLSFCRKTCGTC

SEQ ID NO: 13 - protein, 35 aa, Artificial Sequence
  ShK-Based Peptide
RACIDTIPKSRCTAFQCKHSMKYRLSFCRKTCGTC

SEQ ID NO: 14 - protein, 35 aa, Artificial Sequence
  ShK-Based Peptide
RTCIDTIPKSRCTAFQCKHSMKYRLSFCRKTCGTC

SEQ ID NO: 15 - protein, 35 aa, Artificial Sequence
  ShK-Based Peptide
AQCIDTIPKSRCTAFQCKHSMKYRLSFCRKTCGTC

SEQ ID NO: 16 - protein, 35 aa, Artificial Sequence
  ShK-Based Peptide
AACIDTIPKSRCTAFQCKHSMKYRLSFCRKTCGTC

SEQ ID NO: 17 - protein, 35 aa, Artificial Sequence
  ShK-Based Peptide
ATCIDTIPKSRCTAFQCKHSMKYRLSFCRKTCGTC

SEQ ID NO: 18 - protein, 35 aa, Artificial Sequence
  ShK-Based Peptide
RSCADTIPKSRCTAFQCKHSMKYRLSFCRKTCGTC

SEQ ID NO: 19 - protein, 35 aa, Artificial Sequence
  ShK-Based Peptide
RSCADTIPKSRCTAAQCKHSMKYRLSFCRKTCGTC

SEQ ID NO: 20 - protein, 35 aa, Artificial Sequence
  ShK-Based Peptide
RSCADTIPKSRCTAAQCKHSMKYRASFCRKTCGTC

SEQ ID NO: 21 - protein, 35 aa, Artificial Sequence
  ShK-Based Peptide
RSCIDAIPKSRCTAFQCKHSMKYRLSFCRKTCGTC

SEQ ID NO: 22 - protein, 35 aa, Artificial Sequence; identical to SEQ ID NO: 1
  ShK-Based Peptide
  MOD_RES (35)..(35): AMIDATION

SEQ ID NO: 23 - protein, 35 aa, Artificial Sequence
  ShK-Based Peptide
RSCIDYIPKSRCTAFQCKHSMKYRLSFCRKTCGTC

SEQ ID NO: 24 - protein, 35 aa, Artificial Sequence
  ShK-Based Peptide
RSCIDLIPKSRCTAFQCKHSMKYRLSFCRKTCGTC

SEQ ID NO: 25 - protein, 35 aa, Artificial Sequence
  ShK-Based Peptide
RSCIDTAPKSRCTAFQCKHSMKYRLSFCRKTCGTC

SEQ ID NO: 26 - protein, 35 aa, Artificial Sequence; identical to SEQ ID NO: 18
  ShK-Based Peptide

SEQ ID NO: 27 - protein, 35 aa, Artificial Sequence
  ShK-Based Peptide
RSCIDTIAKSRCTAFQCKHSMKYRLSFCRKTCGTC

SEQ ID NO: 28 - protein, 35 aa, Artificial Sequence
  ShK-Based Peptide
RSCIDTIPASRCTAFQCKHSMKYRLSFCRKTCGTC

SEQ ID NO: 29 - protein, 35 aa, Artificial Sequence
  ShK-Based Peptide
RSCIDTIPESRCTAFQCKHSMKYRLSFCRKTCGTC

SEQ ID NO: 30 - protein, 35 aa, Artificial Sequence
  ShK-Based Peptide
RSCIDTIPQSRCTAFQCKHSMKYRLSFCRKTCGTC

SEQ ID NO: 31 - protein, 35 aa, Artificial Sequence
  ShK-Based Peptide
RSCIDTIPKARCTAFQCKHSMKYRLSFCRKTCGTC

SEQ ID NO: 32 - protein, 35 aa, Artificial Sequence
  ShK-Based Peptide
RSCIDTIPKSACTAFQCKHSMKYRLSFCRKTCGTC

SEQ ID NO: 33 - protein, 35 aa, Artificial Sequence
  ShK-Based Peptide
RSCIDTIPKSECTAFQCKHSMKYRLSFCRKTCGTC

SEQ ID NO: 34 - protein, 35 aa, Artificial Sequence
  ShK-Based Peptide
RSCIDTIPKSQCTAFQCKHSMKYRLSFCRKTCGTC

SEQ ID NO: 35 - protein, 35 aa, Artificial Sequence
  ShK-Based Peptide
RSCIDTIPKSRCAAFQCKHSMKYRLSFCRKTCGTC

SEQ ID NO: 36 - protein, 35 aa, Artificial Sequence
  ShK-Based Peptide
RSCIDTIPKSRCTAAQCKHSMKYRLSFCRKTCGTC

SEQ ID NO: 37 - protein, 35 aa, Artificial Sequence
  ShK-Based Peptide
RSCIDTIPKSRCTAWQCKHSMKYRLSFCRKTCGTC

SEQ ID NO: 38 - protein, 35 aa, Artificial Sequence
  ShK-Based Peptide
  MOD_RES (15)..(15): Any nonfunctional amino acid residue
RSCIDTIPKSRCTAXQCKHSMKYRLSFCRKTCGTC

SEQ ID NO: 39 - protein, 35 aa, Artificial Sequence
  ShK-Based Peptide
RSCIDTIPKSRCTAAQCKHSMKYRASFCRKTCGTC

SEQ ID NO: 40 - protein, 35 aa, Artificial Sequence
  ShK-Based Peptide
RSCIDTIPKSRCTAFACKHSMKYRLSFCRKTCGTC

SEQ ID NO: 41 - protein, 35 aa, Artificial Sequence
  ShK-Based Peptide
RSCIDTIPKSRCTAFECKHSMKYRLSFCRKTCGTC

SEQ ID NO: 42 - protein, 35 aa, Artificial Sequence
  ShK-Based Peptide
RSCIDTIPKSRCTAFQCAHSMKYRLSFCRKTCGTC

SEQ ID NO: 43 - protein, 35 aa, Artificial Sequence
  ShK-Based Peptide
RSCIDTIPKSRCTAFQCEHSMKYRLSFCRKTCGTC

SEQ ID NO: 44 - protein, 35 aa, Artificial Sequence
  ShK-Based Peptide
RSCIDTIPKSRCTAFQCKASMKYRLSFCRKTCGTC

SEQ ID NO: 45 - protein, 35 aa, Artificial Sequence
  ShK-Based Peptide
RSCIDTIPKSRCTAFQCKKSMKYRLSFCRKTCGTC

SEQ ID NO: 46 - protein, 35 aa, Artificial Sequence
  ShK-Based Peptide
RSCIDTIPKSRCTAFQCKHAMKYRLSFCRKTCGTC

SEQ ID NO: 47 - protein, 35 aa, Artificial Sequence
  ShK-Based Peptide
RSCIDTIPKSRCTAFQCKHSAKYRLSFCRKTCGTC

SEQ ID NO: 48 - protein, 35 aa, Artificial Sequence
  ShK-Based Peptide
  MOD_RES (21)..(21): Any nonfunctional amino acid residue
RSCIDTIPKSRCTAFQCKHSXKYRLSFCRKTCGTC

SEQ ID NO: 49 - protein, 35 aa, Artificial Sequence; identical to SEQ ID NO: 48
  ShK-Based Peptide
  MOD_RES (21)..(21): Nle

SEQ ID NO: 50 - protein, 35 aa, Artificial Sequence
  ShK-Based Peptide
RSCIDTIPKSRCTAFQCKHSMAYRLSFCRKTCGTC

SEQ ID NO: 51 - protein, 35 aa, Artificial Sequence
  ShK-Based Peptide
RSCIDTIPKSRCTAFQCKHSMEYRLSFCRKTCGTC

SEQ ID NO: 52 - protein, 35 aa, Artificial Sequence
  ShK-Based Peptide
RSCIDTIPKSRCTAFQCKHSMRYRLSFCRKTCGTC

SEQ ID NO: 53 - protein, 35 aa, Artificial Sequence
  ShK-Based Peptide
  MOD_RES (22)..(22): Any nonfunctional amino acid residue
RSCIDTIPKSRCTAFQCKHSMXYRLSFCRKTCGTC

SEQ ID NO: 54 - protein, 35 aa, Artificial Sequence; identical to SEQ ID NO: 53
  ShK-Based Peptide
  MOD_RES (22)..(22): Nle

SEQ ID NO: 55 - protein, 35 aa, Artificial Sequence; identical to SEQ ID NO: 53
  ShK-Based Peptide
  MOD_RES (22)..(22): Orn

SEQ ID NO: 56 - protein, 35 aa, Artificial Sequence; identical to SEQ ID NO: 53
  ShK-Based Peptide
  MOD_RES (22)..(22): Homocitrulline

SEQ ID NO: 57 - protein, 35 aa, Artificial Sequence; identical to SEQ ID NO: 53
  ShK-Based Peptide
  MISC_FEATURE (22)..(22): Diaminopropionic acid

SEQ ID NO: 58 - protein, 35 aa, Artificial Sequence
  ShK-Based Peptide
RSCIDTIPKSRCTAFQCKHSMKARLSFCRKTCGTC

SEQ ID NO: 59 - protein, 35 aa, Artificial Sequence
  ShK-Based Peptide
RSCIDTIPKSRCTAFQCKHSMKSRLSFCRKTCGTC

SEQ ID NO: 60 - protein, 35 aa, Artificial Sequence
  ShK-Based Peptide
RSCIDTIPKSRCTAFQCKHSMKFRLSFCRKTCGTC

SEQ ID NO: 61 - protein, 35 aa, Artificial Sequence
  ShK-Based Peptide
  MOD_RES (23)..(23): Any nonfunctional amino acid residue
RSCIDTIPKSRCTAFQCKHSMKXRLSFCRKTCGTC

SEQ ID NO: 62 - protein, 35 aa, Artificial Sequence; identical to SEQ ID NO: 61
  ShK-Based Peptide
  MOD_RES (23)..(23): Nitrophenylalanine

SEQ ID NO: 63 - protein, 36 aa, Artificial Sequence
  ShK-Based Peptide
  MOD_RES (23)..(23): Aminophenylalanine
RSCIDTIPKSRCTAFQCKHSMKXRLSFCRKTCGTCC

SEQ ID NO: 64 - protein, 35 aa, Artificial Sequence; identical to SEQ ID NO: 61
  ShK-Based Peptide
  MOD_RES (23)..(23): Benzylphenylalanine

SEQ ID NO: 65 - protein, 35 aa, Artificial Sequence
  ShK-Based Peptide
RSCIDTIPKSRCTAFQCKHSMKYALSFCRKTCGTC

SEQ ID NO: 66 - protein, 35 aa, Artificial Sequence
  ShK-Based Peptide
RSCIDTIPKSRCTAFQCKHSMKYELSFCRKTCGTC

SEQ ID NO: 67 - protein, 35 aa, Artificial Sequence
  ShK-Based Peptide
RSCIDTIPKSRCTAFQCKHSMKYRASFCRKTCGTC

SEQ ID NO: 68 - protein, 35 aa, Artificial Sequence
  ShK-Based Peptide
RSCIDTIPKSRCTAFQCKHSMKYRLAFCRKTCGTC

SEQ ID NO: 69 - protein, 35 aa, Artificial Sequence
  ShK-Based Peptide
RSCIDTIPKSRCTAFQCKHSMKYRLSACRKTCGTC

SEQ ID NO: 70 - protein, 35 aa, Artificial Sequence
  ShK-Based Peptide
  MOD_RES (27)..(27): Any nonfunctional amino acid residue
RSCIDTIPKSRCTAFQCKHSMKYRLSXCRKTCGTC

SEQ ID NO: 71 - protein, 35 aa, Artificial Sequence
  ShK-Based Peptide
RSCIDTIPKSRCTAFQCKHSMKYRLSFCAKTCGTC

SEQ ID NO: 72 - protein, 35 aa, Artificial Sequence
  ShK-Based Peptide
RSCIDTIPKSRCTAFQCKHSMKYRLSFCRATCGTC

SEQ ID NO: 73 - protein, 35 aa, Artificial Sequence
  ShK-Based Peptide
RSCIDTIPKSRCTAFQCKHSMKYRLSFCRKACGTC

SEQ ID NO: 74 - protein, 35 aa, Artificial Sequence
  ShK-Based Peptide
RSCIDTIPKSRCTAFQCKHSMKYRLSFCRKTCGAC

SEQ ID NO: 75 - protein, 34 aa, Artificial Sequence
  ShK-Based Peptide
SCADTIPKSRCTAFQCKHSMKYRLSFCRKTCGTC

SEQ ID NO: 76 - protein, 34 aa, Artificial Sequence
  ShK-Based Peptide
SCADTIPKSRCTAAQCKHSMKYRLSFCRKTCGTC

SEQ ID NO: 77 - protein, 34 aa, Artificial Sequence
  ShK-Based Peptide
SCADTIPKSRCTAAQCKHSMKYRASFCRKTCGTC

SEQ ID NO: 78 - protein, 34 aa, Artificial Sequence
  ShK-Based Peptide
SCIDAIPKSRCTAFQCKHSMKYRLSFCRKTCGTC

SEQ ID NO: 79 - protein, 34 aa, Artificial Sequence
  ShK-Based Peptide
SCIDTAPKSRCTAFQCKHSMKYRLSFCRKTCGTC

SEQ ID NO: 80 - protein, 34 aa, Artificial Sequence
  ShK-Based Peptide
SCIDTIAKSRCTAFQCKHSMKYRLSFCRKTCGTC

SEQ ID NO: 81 - protein, 34 aa, Artificial Sequence
  ShK-Based Peptide
SCIDTIPASRCTAFQCKHSMKYRLSFCRKTCGTC

SEQ ID NO: 82 - protein, 34 aa, Artificial Sequence
  ShK-Based Peptide
SCIDTIPESRCTAFQCKHSMKYRLSFCRKTCGTC

SEQ ID NO: 83 - protein, 34 aa, Artificial Sequence
  ShK-Based Peptide
SCIDTIPQSRCTAFQCKHSMKYRLSFCRKTCGTC

SEQ ID NO: 84 - protein, 34 aa, Artificial Sequence
  ShK-Based Peptide
SCIDTIPKARCTAFQCKHSMKYRLSFCRKTCGTC

SEQ ID NO: 85 - protein, 34 aa, Artificial Sequence
  ShK-Based Peptide
SCIDTIPKSACTAFQCKHSMKYRLSFCRKTCGTC

SEQ ID NO: 86 - protein, 34 aa, Artificial Sequence
  ShK-Based Peptide
SCIDTIPKSECTAFQCKHSMKYRLSFCRKTCGTC

SEQ ID NO: 87 - protein, 34 aa, Artificial Sequence
  ShK-Based Peptide
SCIDTIPKSQCTAFQCKHSMKYRLSFCRKTCGTC

SEQ ID NO: 88 - protein, 34 aa, Artificial Sequence
  ShK-Based Peptide
SCIDTIPKSRCAAFQCKHSMKYRLSFCRKTCGTC

SEQ ID NO: 89 - protein, 34 aa, Artificial Sequence
  ShK-Based Peptide
SCIDTIPKSRCTAAQCKHSMKYRLSFCRKTCGTC

SEQ ID NO: 90 - protein, 34 aa, Artificial Sequence
  ShK-Based Peptide
SCIDTIPKSRCTAWQCKHSMKYRLSFCRKTCGTC

SEQ ID NO: 91 - protein, 34 aa, Artificial Sequence
  ShK-Based Peptide
  MOD_RES (14)..(14): Any nonfunctional amino acid residue
SCIDTIPKSRCTAXQCKHSMKYRLSFCRKTCGTC

SEQ ID NO: 92 - protein, 34 aa, Artificial Sequence
  ShK-Based Peptide
SCIDTIPKSRCTAAQCKHSMKYRASFCRKTCGTC

SEQ ID NO: 93 - protein, 34 aa, Artificial Sequence
  ShK-Based Peptide
SCIDTIPKSRCTAFACKHSMKYRLSFCRKTCGTC

SEQ ID NO: 94 - protein, 34 aa, Artificial Sequence
  ShK-Based Peptide
SCIDTIPKSRCTAFECKHSMKYRLSFCRKTCGTC

SEQ ID NO: 95 - protein, 34 aa, Artificial Sequence
  ShK-Based Peptide
SCIDTIPKSRCTAFQCAHSMKYRLSFCRKTCGTC

SEQ ID NO: 96 - protein, 34 aa, Artificial Sequence
  ShK-Based Peptide
SCIDTIPKSRCTAFQCEHSMKYRLSFCRKTCGTC

SEQ ID NO: 97 - protein, 34 aa, Artificial Sequence
  ShK-Based Peptide
SCIDTIPKSRCTAFQCKASMKYRLSFCRKTCGTC

SEQ ID NO: 98 - protein, 34 aa, Artificial Sequence
  ShK-Based Peptide
SCIDTIPKSRCTAFQCKKSMKYRLSFCRKTCGTC

SEQ ID NO: 99 - protein, 34 aa, Artificial Sequence
  ShK-Based Peptide
SCIDTIPKSRCTAFQCKHAMKYRLSFCRKTCGTC

SEQ ID NO: 100 - protein, 34 aa, Artificial Sequence
  ShK-Based Peptide
SCIDTIPKSRCTAFQCKHSAKYRLSFCRKTCGTC

SEQ ID NO: 101 - protein, 34 aa, Artificial Sequence
  ShK-Based Peptide
  MOD_RES (20)..(20): Any nonfunctional amino acid residue
SCIDTIPKSRCTAFQCKHSXKYRLSFCRKTCGTC

SEQ ID NO: 102 - protein, 34 aa, Artificial Sequence; identical to SEQ ID NO: 101
  ShK-Based Peptide
  MOD_RES (20)..(20): Nle

SEQ ID NO: 103 - protein, 34 aa, Artificial Sequence
  ShK-Based Peptide
SCIDTIPKSRCTAFQCKHSMAYRLSFCRKTCGTC

SEQ ID NO: 104 - protein, 34 aa, Artificial Sequence
  ShK-Based Peptide
SCIDTIPKSRCTAFQCKHSMEYRLSFCRKTCGTC

SEQ ID NO: 105 - protein, 34 aa, Artificial Sequence
  ShK-Based Peptide
SCIDTIPKSRCTAFQCKHSMRYRLSFCRKTCGTC

SEQ ID NO: 106 - protein, 34 aa, Artificial Sequence
  ShK-Based Peptide
  MOD_RES (21)..(21): Any nonfunctional amino acid residue
SCIDTIPKSRCTAFQCKHSMXYRLSFCRKTCGTC

SEQ ID NO: 107 - protein, 34 aa, Artificial Sequence; identical to SEQ ID NO: 106
  ShK-Based Peptide
  MOD_RES (21)..(21): Nle

SEQ ID NO: 108 - protein, 34 aa, Artificial Sequence; identical to SEQ ID NO: 106
  ShK-Based Peptide
  MOD_RES (21)..(21): Orn

SEQ ID NO: 109 - protein, 34 aa, Artificial Sequence; identical to SEQ ID NO: 106
  ShK-Based Peptide
  MOD_RES (21)..(21): Homocitrulline

SEQ ID NO: 110 - protein, 34 aa, Artificial Sequence; identical to SEQ ID NO: 106
  ShK-Based Peptide
  MISC_FEATURE (21)..(21): Diaminopropionic acid

SEQ ID NO: 111 - protein, 34 aa, Artificial Sequence
  ShK-Based Peptide
SCIDTIPKSRCTAFQCKHSMKARLSFCRKTCGTC

SEQ ID NO: 112 - protein, 34 aa, Artificial Sequence
  ShK-Based Peptide
SCIDTIPKSRCTAFQCKHSMKSRLSFCRKTCGTC

SEQ ID NO: 113 - protein, 34 aa, Artificial Sequence
  ShK-Based Peptide
SCIDTIPKSRCTAFQCKHSMKFRLSFCRKTCGTC

SEQ ID NO: 114 - protein, 34 aa, Artificial Sequence
  ShK-Based Peptide
  MOD_RES (22)..(22): Any nonfunctional amino acid residue
SCIDTIPKSRCTAFQCKHSMKXRLSFCRKTCGTC

SEQ ID NO: 115 - protein, 34 aa, Artificial Sequence; identical to SEQ ID NO: 114
  ShK-Based Peptide
  MOD_RES (22)..(22): Nitrophenylalanine

SEQ ID NO: 116 - protein, 34 aa, Artificial Sequence; identical to SEQ ID NO: 114
  ShK-Based Peptide
  MOD_RES (22)..(22): Aminophenylalanine

SEQ ID NO: 117 - protein, 33 aa, Artificial Sequence
  ShK-Based Peptide
  MOD_RES (22)..(22): Benzylphenylalanine
SCIDTIPKSRCTAFQCKHSMKXRLSFCRKTCGT

SEQ ID NO: 118 - protein, 34 aa, Artificial Sequence
  ShK-Based Peptide
SCIDTIPKSRCTAFQCKHSMKYALSFCRKTCGTC

SEQ ID NO: 119 - protein, 34 aa, Artificial Sequence
  ShK-Based Peptide
SCIDTIPKSRCTAFQCKHSMKYELSFCRKTCGTC

SEQ ID NO: 120 - protein, 34 aa, Artificial Sequence
  ShK-Based Peptide
SCIDTIPKSRCTAFQCKHSMKYRASFCRKTCGTC

SEQ ID NO: 121 - protein, 34 aa, Artificial Sequence
  ShK-Based Peptide
SCIDTIPKSRCTAFQCKHSMKYRLAFCRKTCGTC

SEQ ID NO: 122 - protein, 34 aa, Artificial Sequence
  ShK-Based Peptide
SCIDTIPKSRCTAFQCKHSMKYRLSACRKTCGTC

SEQ ID NO: 123 - protein, 34 aa, Artificial Sequence
  ShK-Based Peptide
  MOD_RES (26)..(26): Any nonfunctional amino acid residue
SCIDTIPKSRCTAFQCKHSMKYRLSXCRKTCGTC

SEQ ID NO: 124 - protein, 34 aa, Artificial Sequence
  ShK-Based Peptide
SCIDTIPKSRCTAFQCKHSMKYRLSFCAKTCGTC

SEQ ID NO: 125 - protein, 34 aa, Artificial Sequence
  ShK-Based Peptide
SCIDTIPKSRCTAFQCKHSMKYRLSFCRATCGTC

SEQ ID NO: 126 - protein, 34 aa, Artificial Sequence
  ShK-Based Peptide
SCIDTIPKSRCTAFQCKHSMKYRLSFCRKACGTC

SEQ ID NO: 127 - protein, 34 aa, Artificial Sequence
  ShK-Based Peptide
SCIDTIPKSRCTAFQCKHSMKYRLSFCRKTCGAC

SEQ ID NO: 128 - protein, 35 aa, Artificial Sequence
  ShK-Based Peptide
YSCIDTIPKSRCTAFQCKHSMKYRLSFCRKTCGTC

SEQ ID NO: 129 - protein, 35 aa, Artificial Sequence
  ShK-Based Peptide
KSCIDTIPKSRCTAFQCKHSMKYRLSFCRKTCGTC

SEQ ID NO: 130 - protein, 35 aa, Artificial Sequence
  ShK-Based Peptide
HSCIDTIPKSRCTAFQCKHSMKYRLSFCRKTCGTC

SEQ ID NO: 131 - protein, 35 aa, Artificial Sequence
  ShK-Based Peptide
QSCIDTIPKSRCTAFQCKHSMKYRLSFCRKTCGTC

SEQ ID NO: 132 - protein, 37 aa, Artificial Sequence
  ShK-Based Peptide
PPRSCIDTIPKSRCTAFQCKHSMKYRLSFCRKTCGTC

SEQ ID NO: 133 - protein, 36 aa, Artificial Sequence
  ShK-Based Peptide
MRSCIDTIPKSRCTAFQCKHSMKYRLSFCRKTCGTC

SEQ ID NO: 134 - protein, 36 aa, Artificial Sequence
  ShK-Based Peptide
GRSCIDTIPKSRCTAFQCKHSMKYRLSFCRKTCGTC

SEQ ID NO: 135 - protein, 35 aa, Artificial Sequence
  ShK-Based Peptide
YSCIDTIPKSRCTAFQCKHSMAYRLSFCRKTCGTC

SEQ ID NO: 136 - protein, 35 aa, Artificial Sequence
  ShK-Based Peptide
KSCIDTIPKSRCTAFQCKHSMAYRLSFCRKTCGTC

SEQ ID NO: 137 - protein, 35 aa, Artificial Sequence
  ShK-Based Peptide
HSCIDTIPKSRCTAFQCKHSMAYRLSFCRKTCGTC

SEQ ID NO: 138 - protein, 35 aa, Artificial Sequence
  ShK-Based Peptide
QSCIDTIPKSRCTAFQCKHSMAYRLSFCRKTCGTC

SEQ ID NO: 139 - protein, 37 aa, Artificial Sequence
  ShK-Based Peptide
PPRSCIDTIPKSRCTAFQCKHSMAYRLSFCRKTCGTC

SEQ ID NO: 140 - protein, 36 aa, Artificial Sequence
  ShK-Based Peptide
MRSCIDTIPKSRCTAFQCKHSMAYRLSFCRKTCGTC

SEQ ID NO: 141 - protein, 36 aa, Artificial Sequence
  ShK-Based Peptide
GRSCIDTIPKSRCTAFQCKHSMAYRLSFCRKTCGTC

SEQ ID NO: 142 - protein, 35 aa, Artificial Sequence
  ShK-Based Peptide
RSCIDTIPASRCTAFQCKHSMAYRLSFCRKTCGTC

SEQ ID NO: 143 - protein, 34 aa, Artificial Sequence
  ShK-Based Peptide
SCIDTIPASRCTAFQCKHSMAYRLSFCRKTCGTC

SEQ ID NO: 144 - protein, 35 aa, Artificial Sequence
  ShK-Based Peptide
RSCIDTIPVSRCTAFQCKHSMKYRLSFCRKTCGTC

SEQ ID NO: 145 - protein, 35 aa, Artificial Sequence
  ShK-Based Peptide
RSCIDTIPVSRCTAFQCKHSMAYRLSFCRKTCGTC

SEQ ID NO: 146 - protein, 34 aa, Artificial Sequence
  ShK-Based Peptide
SCIDTIPVSRCTAFQCKHSMKYRLSFCRKTCGTC

SEQ ID NO: 147 - protein, 34 aa, Artificial Sequence
  ShK-Based Peptide
SCIDTIPVSRCTAFQCKHSMAYRLSFCRKTCGTC

SEQ ID NO: 148 - protein, 35 aa, Artificial Sequence
  ShK-Based Peptide
RSCIDTIPESRCTAFQCKHSMAYRLSFCRKTCGTC

SEQ ID NO: 149 - protein, 34 aa, Artificial Sequence
  ShK-Based Peptide
SCIDTIPESRCTAFQCKHSMAYRLSFCRKTCGTC

SEQ ID NO: 150 - protein, 35 aa, Artificial Sequence
  ShK-Based Peptide
RSCIDTIPKSACTAFQCKHSMAYRLSFCRKTCGTC

SEQ ID NO: 151 - protein, 34 aa, Artificial Sequence
  ShK-Based Peptide
SCIDTIPKSACTAFQCKHSMAYRLSFCRKTCGTC

SEQ ID NO: 152 - protein, 35 aa, Artificial Sequence
  ShK-Based Peptide
RSCIDTIPKSECTAFQCKHSMAYRLSFCRKTCGTC

SEQ ID NO: 153 - protein, 34 aa, Artificial Sequence
  ShK-Based Peptide
SCIDTIPKSECTAFQCKHSMAYRLSFCRKTCGTC

SEQ ID NO: 154 - protein, 35 aa, Artificial Sequence
  ShK-Based Peptide
RSCIDTIPKSRCTDFQCKHSMKYRLSFCRKTCGTC

SEQ ID NO: 155 - protein, 35 aa, Artificial Sequence
  ShK-Based Peptide
RSCIDTIPKSRCTDFQCKHSMAYRLSFCRKTCGTC

SEQ ID NO: 156 - protein, 34 aa, Artificial Sequence
  ShK-Based Peptide
SCIDTIPKSRCTDFQCKHSMKYRLSFCRKTCGTC

SEQ ID NO: 157 - protein, 34 aa, Artificial Sequence
  ShK-Based Peptide
SCIDTIPKSRCTDFQCKHSMAYRLSFCRKTCGTC

SEQ ID NO: 158 - protein, 35 aa, Artificial Sequence
  ShK-Based Peptide
RSCIDTIPKSRCTAAQCKHSMAYRLSFCRKTCGTC

SEQ ID NO: 159 - protein, 34 aa, Artificial Sequence
  ShK-Based Peptide
SCIDTIPKSRCTAAQCKHSMAYRLSFCRKTCGTC

SEQ ID NO: 160 - protein, 35 aa, Artificial Sequence
  ShK-Based Peptide
RSCIDTIPKSRCTAIQCKHSMKYRLSFCRKTCGTC

SEQ ID NO: 161 - protein, 35 aa, Artificial Sequence
  ShK-Based Peptide
RSCIDTIPKSRCTAIQCKHSMAYRLSFCRKTCGTC

SEQ ID NO: 162 - protein, 34 aa, Artificial Sequence
  ShK-Based Peptide
SCIDTIPKSRCTAIQCKHSMKYRLSFCRKTCGTC

SEQ ID NO: 163 - protein, 34 aa, Artificial Sequence
  ShK-Based Peptide
SCIDTIPKSRCTAIQCKHSMAYRLSFCRKTCGTC

SEQ ID NO: 164 - protein, 35 aa, Artificial Sequence
  ShK-Based Peptide
RSCIDTIPKSRCTAVQCKHSMKYRLSFCRKTCGTC

SEQ ID NO: 165 - protein, 35 aa, Artificial Sequence
  ShK-Based Peptide
RSCIDTIPKSRCTAVQCKHSMAYRLSFCRKTCGTC

SEQ ID NO: 166 - protein, 34 aa, Artificial Sequence
  ShK-Based Peptide
SCIDTIPKSRCTAVQCKHSMKYRLSFCRKTCGTC

SEQ ID NO: 167 - protein, 34 aa, Artificial Sequence
  ShK-Based Peptide
SCIDTIPKSRCTAVQCKHSMAYRLSFCRKTCGTC

SEQ ID NO: 168 - protein, 35 aa, Artificial Sequence
  ShK-Based Peptide
RSCIDTIPKSRCTAFRCKHSMKYRLSFCRKTCGTC

SEQ ID NO: 169 - protein, 35 aa, Artificial Sequence
  ShK-Based Peptide
RSCIDTIPKSRCTAFRCKHSMAYRLSFCRKTCGTC

SEQ ID NO: 170 - protein, 34 aa, Artificial Sequence
  ShK-Based Peptide
SCIDTIPKSRCTAFRCKHSMKYRLSFCRKTCGTC

SEQ ID NO: 171 - protein, 34 aa, Artificial Sequence
  ShK-Based Peptide
SCIDTIPKSRCTAFRCKHSMAYRLSFCRKTCGTC

SEQ ID NO: 172 - protein, 35 aa, Artificial Sequence
  ShK-Based Peptide
RSCIDTIPKSRCTAFKCKHSMKYRLSFCRKTCGTC

SEQ ID NO: 173 - protein, 35 aa, Artificial Sequence
  ShK-Based Peptide
RSCIDTIPKSRCTAFKCKHSMAYRLSFCRKTCGTC

SEQ ID NO: 174 - protein, 34 aa, Artificial Sequence
  ShK-Based Peptide
SCIDTIPKSRCTAFKCKHSMKYRLSFCRKTCGTC

SEQ ID NO: 175 - protein, 34 aa, Artificial Sequence
  ShK-Based Peptide
SCIDTIPKSRCTAFKCKHSMAYRLSFCRKTCGTC

SEQ ID NO: 176 - protein, 35 aa, Artificial Sequence
  ShK-Based Peptide
RSCIDTIPASECTAFQCKHSMKYRLSFCRKTCGTC

SEQ ID NO: 177 - protein, 35 aa, Artificial Sequence
  ShK-Based Peptide
RSCIDTIPASECTAFQCKHSMAYRLSFCRKTCGTC

SEQ ID NO: 178 - protein, 34 aa, Artificial Sequence
  ShK-Based Peptide
SCIDTIPASECTAFQCKHSMKYRLSFCRKTCGTC

SEQ ID NO: 179 - protein, 34 aa, Artificial Sequence
  ShK-Based Peptide
SCIDTIPASECTAFQCKHSMAYRLSFCRKTCGTC

SEQ ID NO: 180 - protein, 35 aa, Artificial Sequence
  ShK-Based Peptide
RSCIDTIPVSECTAFQCKHSMKYRLSFCRKTCGTC

SEQ ID NO: 181 - protein, 35 aa, Artificial Sequence
  ShK-Based Peptide
RSCIDTIPVSECTAFQCKHSMAYRLSFCRKTCGTC

SEQ ID NO: 182 - protein, 34 aa, Artificial Sequence
  ShK-Based Peptide
SCIDTIPVSECTAFQCKHSMKYRLSFCRKTCGTC

SEQ ID NO: 183 - protein, 34 aa, Artificial Sequence
  ShK-Based Peptide
SCIDTIPVSECTAFQCKHSMAYRLSFCRKTCGTC

SEQ ID NO: 184 - protein, 35 aa, Artificial Sequence
  ShK-Based Peptide
RSCIDTIPVSACTAFQCKHSMKYRLSFCRKTCGTC

SEQ ID NO: 185 - protein, 35 aa, Artificial Sequence
  ShK-Based Peptide
RSCIDTIPVSACTAFQCKHSMAYRLSFCRKTCGTC

SEQ ID NO: 186 - protein, 34 aa, Artificial Sequence
  ShK-Based Peptide
SCIDTIPVSACTAFQCKHSMKYRLSFCRKTCGTC

SEQ ID NO: 187 - protein, 34 aa, Artificial Sequence
  ShK-Based Peptide
SCIDTIPVSACTAFQCKHSMAYRLSFCRKTCGTC

SEQ ID NO: 188 - protein, 35 aa, Artificial Sequence
  ShK-Based Peptide
RSCIDTIPASACTAFQCKHSMKYRLSFCRKTCGTC

SEQ ID NO: 189 - protein, 35 aa, Artificial Sequence
  ShK-Based Peptide
RSCIDTIPASACTAFQCKHSMAYRLSFCRKTCGTC

SEQ ID NO: 190 - protein, 34 aa, Artificial Sequence
  ShK-Based Peptide
SCIDTIPASACTAFQCKHSMKYRLSFCRKTCGTC

SEQ ID NO: 191 - protein, 34 aa, Artificial Sequence
  ShK-Based Peptide
SCIDTIPASACTAFQCKHSMAYRLSFCRKTCGTC

SEQ ID NO: 192 - protein, 35 aa, Artificial Sequence
  ShK-Based Peptide
RSCIDTIPKSECTDIRCKHSMKYRLSFCRKTCGTC

SEQ ID NO: 193 - protein, 35 aa, Artificial Sequence
  ShK-Based Peptide
RSCIDTIPKSECTDIRCKHSMAYRLSFCRKTCGTC

SEQ ID NO: 194 - protein, 34 aa, Artificial Sequence
  ShK-Based Peptide
SCIDTIPKSECTDIRCKHSMKYRLSFCRKTCGTC

SEQ ID NO: 195 - protein, 34 aa, Artificial Sequence
  ShK-Based Peptide
SCIDTIPKSECTDIRCKHSMAYRLSFCRKTCGTC

SEQ ID NO: 196 - protein, 35 aa, Artificial Sequence
  ShK-Based Peptide
RSCIDTIPVSECTDIRCKHSMKYRLSFCRKTCGTC

SEQ ID NO: 197 - protein, 35 aa, Artificial Sequence
  ShK-Based Peptide
RSCIDTIPVSECTDIRCKHSMAYRLSFCRKTCGTC

SEQ ID NO: 198 - protein, 34 aa, Artificial Sequence
  ShK-Based Peptide
SCIDTIPVSECTDIRCKHSMKYRLSFCRKTCGTC

SEQ ID NO: 199 - protein, 34 aa, Artificial Sequence
  ShK-Based Peptide
SCIDTIPVSECTDIRCKHSMAYRLSFCRKTCGTC

SEQ ID NO: 200 - protein, 35 aa, Artificial Sequence
  ShK-Based Peptide
RSCIDTIPVSECTDIQCKHSMKYRLSFCRKTCGTC

SEQ ID NO: 201 - protein, 35 aa, Artificial Sequence
  ShK-Based Peptide
RSCIDTIPVSECTDIQCKHSMAYRLSFCRKTCGTC

SEQ ID NO: 202 - protein, 34 aa, Artificial Sequence
  ShK-Based Peptide
SCIDTIPVSECTDIQCKHSMKYRLSFCRKTCGTC

SEQ ID NO: 203 - protein, 34 aa, Artificial Sequence
  ShK-Based Peptide
SCIDTIPVSECTDIQCKHSMAYRLSFCRKTCGTC

SEQ ID NO: 204 - protein, 35 aa, Artificial Sequence
  ShK-Based Peptide
RTCKDLIPVSECTDIRCKHSMKYRLSFCRKTCGTC

SEQ ID NO: 205 - protein, 35 aa, Artificial Sequence
  ShK-Based Peptide
RTCKDLIPVSECTDIRCKHSMAYRLSFCRKTCGTC

SEQ ID NO: 206 - protein, 34 aa, Artificial Sequence
  ShK-Based Peptide
TCKDLIPVSECTDIRCKHSMKYRLSFCRKTCGTC

SEQ ID NO: 207 - protein, 34 aa, Artificial Sequence
  ShK-Based Peptide
TCKDLIPVSECTDIRCKHSMAYRLSFCRKTCGTC

SEQ ID NO: 208 - protein, 37 aa, Artificial Sequence
  ShK-Based Peptide
  MOD_RES (1)..(1): L-Phosphotyrosine
  MISC_FEATURE (2)..(2): Aminoethyloxyethyloxyacetic acid
XXRSCIDTIPKSRCTAFQCKHSMKYRLSFCRKTCGTC

SEQ ID NO: 209 - protein, 37 aa, Artificial Sequence; identical to SEQ ID NO: 208
  ShK-Based Peptide
  MOD_RES (1)..(1): L-Tyrosine
  MISC_FEATURE (2)..(2): Aminoethyloxyethyloxyacetic acid

SEQ ID NO: 210 - protein, 37 aa, Artificial Sequence; identical to SEQ ID NO: 208
  ShK-Based Peptide
  MOD_RES (1)..(1): L-Tyrosine
  MISC_FEATURE (2)..(2): Aminoethyloxyethyloxyacetic acid
  MOD_RES (37)..(37): AMIDATION

SEQ ID NO: 211 - protein, 35 aa, Artificial Sequence
  ShK-Based Peptide
QSCADTIPKSRCTAAQCKHSMKYRLSFCRKTCGTC

SEQ ID NO: 212 - protein, 35 aa, Artificial Sequence
  ShK-Based Peptide
QSCADTIPKSRCTAAQCKHSMAYRLSFCRKTCGTC

SEQ ID NO: 213 - protein, 35 aa, Artificial Sequence
  ShK-Based Peptide
  MISC_FEATURE (22)..(22): Diaminopropionic acid
QSCADTIPKSRCTAAQCKHSMXYRLSFCRKTCGTC

SEQ ID NO: 214 - protein, 35 aa, Artificial Sequence
  ShK-Based Peptide
QSCADTIPKSRCTAAQCKHSMKYRASFCRKTCGTC

SEQ ID NO: 215 - protein, 35 aa, Artificial Sequence
  ShK-Based Peptide
QSCADTIPKSRCTAAQCKHSMAYRASFCRKTCGTC

SEQ ID NO: 216 - protein, 35 aa, Artificial Sequence
  ShK-Based Peptide
  MISC_FEATURE (22)..(22): Diaminopropionic acid
QSCADTIPKSRCTAAQCKHSMXYRASFCRKTCGTC

SEQ ID NO: 217 - protein, 37 aa, Artificial Sequence; identical to SEQ ID NO: 208
  ShK-Based Peptide
  MOD_RES (1)..(1): L-Phosphotyrosine
  MISC_FEATURE (2)..(2): Aminoethyloxyethyloxyacetic acid
  MOD_RES (37)..(37): AMIDATION

SEQ ID NO: 218 - protein, 37 aa, Artificial Sequence
  ShK-Based Peptide
  MOD_RES (1)..(1): Para-phosphono-phenylalanine
  MISC_FEATURE (2)..(2): Aminoethyloxyethyloxyacetic acid
  MOD_RES (23)..(23): Nle
  MOD_RES (37)..(37): AMIDATION
XXRSCIDTIPKSRCTAFQCKHSXKYRLSFCRKTCGTC

SEQ ID NO: 219 - protein, 37 aa, Artificial Sequence; identical to SEQ ID NO: 208
  ShK-Based Peptide
  MOD_RES (1)..(1): Phosphonomethyl-phenylalanine
  MISC_FEATURE (2)..(2): Aminoethyloxyethyloxyacetic acid
  MOD_RES (37)..(37): AMIDATION

SEQ ID NO: 220 - protein, 37 aa, Artificial Sequence; identical to SEQ ID NO: 218
  ShK-Based Peptide
  MOD_RES (1)..(1): Phosphonomethyl-phenylalanine
  MISC_FEATURE (2)..(2): Aminoethyloxyethyloxyacetic acid
  MOD_RES (23)..(23): Nle
  MOD_RES (37)..(37): AMIDATION

SEQ ID NO: 221 - protein, 39 aa, Artificial Sequence
  ShK-Based Peptide
  MISC_FEATURE (1)..(1): 1,4,7,10-tetraazacyclododecane-1,4,7, 10-tetraacetic acid
  MISC_FEATURE (2)..(2): aminohexanoicacid
  MOD_RES (3)..(3): L-Tyrosine
  MISC_FEATURE (4)..(4): Aminoethyloxyethyloxyacetic acid
  MOD_RES (39)..(39): AMIDATION
XXXXRSCIDTIPKSRCTAFQCKHSMKYRLSFCRKTCGTC

SEQ ID NO: 222 - protein, 37 aa, Artificial Sequence
  ShK-Based Peptide
  MOD_RES (1)..(1): Para-phosphono-Phenylalanine
  MISC_FEATURE (2)..(2): Aminoethyloxyethyloxyacetic acid
  MOD_RES (23)..(23): Nle
  MOD_RES (37)..(37): AMIDATION
XXRSCIDTIPKSRCTAFKCKHSXKYRLSFCRKTCGTC

SEQ ID NO: 223 - protein, 37 aa, Artificial Sequence; identical to SEQ ID NO: 208
  ShK-Based Peptide
  MOD_RES (1)..(1): Para-phosphono-Phenylalanine
  MISC_FEATURE (2)..(2): Aminoethyloxyethyloxyacetic acid
  MOD_RES (37)..(37): AMIDATION

SEQ ID NO: 224 - protein, 35 aa, Artificial Sequence
  ShK-Based Peptide
  MOD_RES (21)..(21): Nle
  MISC_FEATURE (22)..(22): Diaminopropionic acid
RSCIDTIPKSRCTAFQCKHSXXYRLSFCRKTCGTC

SEQ ID NO: 225 - protein, 35 aa, Pandinus imperator
LVKCRGTSDCGRPCQQQTGCPNSKCINRMCKCYGC

SEQ ID NO: 226 - protein, 35 aa, Pandinus imperator
TISCTNPKQCYPHCKKETGYPNAKCMNRKCKCFGR

SEQ ID NO: 227 - protein, 35 aa, Pandinus imperator
TISCTNEKQCYPHCKKETGYPNAKCMNRKCKCFGR

SEQ ID NO: 228 - protein, 38 aa, Pandinus imperator
IEAIRCGGSRDCYRPCQKRTGCPNAKCINKTCKCYGCS

SEQ ID NO: 229 - protein, 34 aa, Heterometrus spinifer
ASCRTPKDCADPCRKETGCPYGKCMNRKCKCNRC

SEQ ID NO: 230 - protein, 38 aa, Leiurus quinquestriatus hebraeus
GVPINVSCTGSPQCIKPCKDAGMRFGKCMNRKCHCTPK

SEQ ID NO: 231 - protein, 38 aa, Leiurus quinquestriatus hebraeus
GVPINVKCTGSPQCLKPCKDAGMRFGKCINGKCHCTPK

SEQ ID NO: 232 - protein, 38 aa, Orthochirus scrobiculosus
GVIINVKCKISRQCLEPCKKAGMRFGKCMNGKCHCTPK

SEQ ID NO: 233 - protein, 35 aa, Anuroctonus phaiodactylus
ZKECTGPQHCTNFCRKNKCTHGKCMNRKCKCFNCK

SEQ ID NO: 234 - protein, 39 aa, Centruroides noxius
TIINVKCTSPKQCSKPCKELYGSSAGAKCMNGKCKCYNN

SEQ ID NO: 235 - protein, 39 aa, Centruroides limbatus
TVIDVKCTSPKQCLPPCKAQFGLRAGAKCMNGKCKCYPH

SEQ ID NO: 236 - protein, 37 aa, Leiurus quinquestriatus hebraeus
QFTNVSCTTSKECWSVCQRLHNTSRGKCMNKKCRCYS

SEQ ID NO: 237 - protein, 37 aa, Tityus serrulatus
VFINAKCRGSPECLPKCKEAIGKAAGKCMNGKCKCYP

SEQ ID NO: 238 - protein, 37 aa, Bunodosoma granuliferum
VCRDWFKETACRHAKSLGNCRTSQKYRANCAKTCELC

SEQ ID NO: 239 - protein, 38 aa, Mesobuthus martensii
VGINVKCKHSGQCLKPCKDAGMRFGKCINGKCDCTPKG

SEQ ID NO: 240 - protein, 37 aa, Mesobuthus martensii
QFTDVKCTGSKQCWPVCKQMFGKPNGKCMNGKCRCYS

SEQ ID NO: 241 - protein, 37 aa, Tityus cambridgei
VFINVKCRGSKECLPACKAAVGKAAGKCMNGKCKCYP

SEQ ID NO: 242 - protein, 35 aa, Tityus cambridgei
TGPQTTCQAAMCEAGCKGLGKSMESCQGDTCKCKA

SEQ ID NO: 243 - protein, 36 aa, Vaejovis mexicanus smithi
  MOD_RES (36)..(36): AMIDATION
AAAISCVGSPECPPKCRAQGCKNGKCMNRKCKCYYC

SEQ ID NO: 244 - protein, 35 aa, Heteractis magnifica
RTCKDLIPVSECTDIRCRTSMKYRLNLCRKTCGSC

SEQ ID NO: 245 - protein, 36 aa, Actinia equina
GCKDNFSANTCKHVKANNNCGSQKYATNCAKTCGKC

SEQ ID NO: 246 - protein, 36 aa, Anemonia sulcata
ACKDNFAAATCKHVKENKNCGSQKYATNCAKTCGKC

SEQ ID NO: 247 - protein, 39 aa, Centruroides margaritatus
TIINVKCTSPKQCLPPCKAQFGQSAGAKCMNGKCKCYPH

SEQ ID NO: 248 - protein, 38 aa, Androctonus mauritanicus mauritanicus
GVEINVKCSGSPQCLKPCKDAGMRFGKCMNRKCHCTPK

SEQ ID NO: 249 - protein, 37 aa, Androctonus australis
VRIPVSCKHSGQCLKPCKDAGMRFGKCMNGKCDCTPK

SEQ ID NO: 250 - protein, 34 aa, Scorpio maurus palmatus
VSCTGSKDCYAPCRKQTGCPNAKCINKSCKCYGC

SEQ ID NO: 251 - protein, 36 aa, Mesobuthus tamulus
QFTDVDCSVSKECWSVCKDLFGVDRGKCMGKKCRCY

SEQ ID NO: 252 - protein, 38 aa, Odontobuthus doriae
GVPTDVKCRGSPQCIQPCKDAGMRFGKCMNGKCHCTPK

SEQ ID NO: 253 - protein, 38 aa, Mesobuthus tamulus sindicus
GVPINVKCRGSPQCIQPCRDAGMRFGKCMNGKCHCTPQ

SEQ ID NO: 254 - protein, 37 aa, Buthus occitanus israelis
GVPINVKCRGSRDCLDPCKKAGMRFGKCINSKCHCTP

SEQ ID NO: 255 - protein, 38 aa, Leiurus quinquestriatus hebraeus
GVPINVPCTGSPQCIKPCKDAGMRFGKCMNRKCHCTPK

SEQ ID NO: 256 - protein, 37 aa, Buthus occitanus tunetanus
VGIPVSCKHSGQCIKPCKDAGMRFGKCMNRKCDCTPK

SEQ ID NO: 257 - protein, 37 aa, Artificial Sequence; identical to SEQ ID NO: 218
  ShK-Based Peptide
  MOD_RES (1)..(1): L-Phosphotyrosine
  MISC_FEATURE (2)..(2): Aminoethyloxyethyloxyacetic acid
  MOD_RES (23)..(23): Norleucine

SEQ ID NO: 258 - protein, 37 aa, Artificial Sequence; identical to SEQ ID NO: 218
  ShK-Based Peptide
  MOD_RES (1)..(1): L-Tyrosine
  MISC_FEATURE (2)..(2): Aminoethyloxyethyloxyacetic acid
  MOD_RES (23)..(23): Norluecine

SEQ ID NO: 259 - protein, 37 aa, Artificial Sequence; identical to SEQ ID NO: 218
  ShK-Based Peptide
  MOD_RES (1)..(1): L-Phosphotyrosine
  MISC_FEATURE (2)..(2): Aminoethyloxyethyloxyacetic acid
  MOD_RES (23)..(23): Norleucine
  MOD_RES (37)..(37): Amidation

SEQ ID NO: 260 - protein, 37 aa, Artificial Sequence; identical to SEQ ID NO: 218
  ShK-Based Peptide
  MOD_RES (1)..(1): L-Tyrosine
  MISC_FEATURE (2)..(2): Aminoethyloxyethyloxyacetic acid
  MOD_RES (23)..(23): Norleucine
  MOD_RES (37)..(37): Amidation

(19) United States
(12) Patent Application Publication
(10) Pub. No.: US 2018/0264080 A1
(43) Pub. Date: Sep. 20, 2018

(54) TOPICAL APPLICATIONS OF Kv1.3 CHANNEL BLOCKING PEPTIDES TO TREAT SKIN INFLAMMATION

(71) Applicant: KINETA ONE, LLC, Seattle, WA (US)

(72) Inventors: Shawn P. Iadonato, Seattle, WA (US); Eric Tarcha, Sammamish, WA (US); Kurt Lustig, Seattle, WA (US)

(73) Assignee: KINETA ONE, LLC, Seattle, WA (US)

(21) Appl. No.: 15/542,289

(22) PCT Filed: Jan. 7, 2016
(86) PCT No.: PCT/US2016/012517

§ 371 Date: Jul. 7, 2017

Related U.S. Application Data
(60) Provisional application No. 62/124,983, filed on Jan. 9, 2015.

(51) Int. Cl. A61K 38/17; A61P 17/00; A61K 9/00

(52) U.S. Cl. CPC A61K 38/1767; A61K 9/0014; A61P 17/00

(57) ABSTRACT

Disclosed herein are methods for the topical treatment of inflammation of the skin and mucosa by applying a pharmaceutical composition including a protein or peptide that blocks the Kv1.3 potassium channel. The proteins and peptides can include an ShK-based protein and peptide.

### FIELD OF THE DISCLOSURE

The methods disclosed herein relate to the topical use of toxin-based peptides and proteins to treat conditions of skin inflammation including psoriasis and atopic dermatitis, among other uses. The toxin-based peptides can include ShK-based peptides. Pharmaceutical compositions including the peptides are also disclosed.

### BACKGROUND

Autoimmune and inflammatory diseases of the skin are common and include psoriasis and atopic dermatitis. Many of the most common forms of immune mediated skin inflammation are caused by pathogenic T cells, and many systemic T cell-mediated autoimmune diseases have associated skin manifestations. In particular T cells have been implicated in the pathogenesis of psoriasis; atopic dermatitis; eczema; skin and mucosal lesions in Bechet's disease, scleroderma, systemic sclerosis, systemic lupus erythematosis, inflammatory bowel disease, Graves' disease, Hashimoto's thyroiditis and graft-versus-host disease; drug induced hypersensitivity reactions; allergic contact dermatitis; vitiligo; dermatomyositis; bullous pemphigoid; pemphigus vulgaris; pemphigus foliaceus; lichen planus; fixed drug eruption; delayed-type hypersensitivity reaction; Vogt-Koyanagi-Harada syndrome; Sjogren's syndrome; alopecia areata; Wiskott-Aldrich syndrome; Stevens-Johnson syndrome; toxic epidermal necrolysis; cutaneous lupus erythematosis; lichenoid tissue reaction; and oral lichen planus. In addition, certain cutaneous T cell lymphomas are associated with aberrant memory T cells including mycosis fungoides and Sézary syndrome.

By far topical therapies are the most commonly used first-line treatment for the cutaneous manifestations of autoimmune disease. In particular topical steroids are widely prescribed to remediate the common inflammatory conditions of the skin. However, topical steroids can lose effectiveness with chronic use and lead to specific dermatological side effects including skin atrophy, rosacea, steroid allergy, skin irritation, and potentially mild Cushing's syndrome from systemic absorption. Second-line topical therapies have been developed for many diseases, and include for example, topical tacrolimus and pimecrolimus, phototherapy, vitamin D analogs, retinoids, and the like. When topical therapies fail, systemic immune modulating drugs, immunosuppressants and chemotherapy can be used to blunt the inflammatory process. However, there is a significant unmet medical need for new topical therapies with novel drug mechanisms.

Studies have shown that normal noninflammatory skin is heavily colonized by long-lasting populations of T cells that provide a first line of defense against infection. Studies of skin-resident T cell populations have demonstrated that most have an effector memory phenotype. These T cells express the CD45RO antigen typical of human memory T cells, but largely lack the CCR7 homing receptor for lymphoid organs. This effector memory T cell population has been shown to play a prominent role in the pathology of skin inflammatory diseases including psoriasis. Activation of effector memory T cells is dependent on the action of the voltage-gated Kv1.3 potassium channel, making this channel an attractive drug target for the treatment of T cell-mediated autoimmune diseases. By far the most effective and selective Kv1.3 channel blockers are derived from venom peptides; however these compounds have thus far only been used in systemic applications following parenteral administration. While topical preparations and uses of very short peptides have been previously described, most Kv1.3 channel blocking peptides, and most peptides and proteins in general, do not have the necessary physiochemical properties for topical, mucosal or transdermal delivery, primarily because of their large molecular size (>1000 daltons), charged nature at physiological pH and hydrophilic properties.

### SUMMARY OF THE DISCLOSURE

A need exists to deliver potent and selective peptide-based Kv1.3 channel blockers locally to the site of inflammation rather than systemically through injection. Local delivery reduces systemic exposure and increases the concentration of the peptide at the intended site of action, reducing off-target effects and enhancing on-target effects. Further a need exists to provide the peptide topically for transderm delivery without the need for physical methods such as injection, iontophoresis, electroporation, sonophoresis, or microneedle devices.

The present disclosure provides methods for formulating and delivering topical preparations of Kv1.3 channel blocking peptides for the treatment of inflammation of the skin and mucosa. Diseases that can benefit from the topical application of Kv1.3 blocking peptides include autoimmune diseases of the skin and mucosa specifically and autoimmune diseases with skin and mucosal manifestations. Therefore the present disclosure provides for methods of and topical compositions for treating cutaneous lupus, dermatomyositis, scleroderma, psoriasis, atopic dermatitis, vasculitis, Bechet's syndrome, Henoch-Schonlein prupura, hypersensitivity reactions, Kawasaki disease, microscopic polyangitis, polyarteritis nodosa, vitiligo, alopecia areata, autoimmune progesterone dermatitis, Henoch-Schonlein purpura, Blau syndrome, bullous penphigoid, Churg-Strauss syndrome, cicatricial pemphigoid, contact dermatitis, Chron's disease, inflammatory bowel disease, dermatitis herpetiformis, diffuse cutaneous systemic sclerosis, discoid lupus erythematosus, ecaema, eosinophilic fasciitis, epidermolysis bullosa acquisita, erythema nodosum, lichen planus, lupus erythematosus, Majeed syndrome, morphea, Mucha-Habermann disease, Parry-Romberg syndrome, POEMS syndrome, psoriasis, pyoderma gangrenosum, relapsing polychondritis, reactive arthritis, rheumatoid arthritis, scleroderma, Sjogren's syndrome, systemic lupus erythematosis, vasculitis and Wegener's granulomatosis.

The disclosure also provides methods for formulating and delivering topical preparations of Kv1.3 channel blocking peptides for the treatment of cutaneous T cell lymphomas.

### BRIEF DESCRIPTION OF THE DRAWINGS

FIGS. 1A and 1B show average clinical scores of animals from 2 separate delayed-type hypersensitivity (DTH) reaction studies (FIG. 1A, FIG. 1B) at 24 hours post-challenge that were treated with either 1% ShK-186 or vehicle (9 mM sodium phosphate, 0.72% NaCl, 0.045% polysorbate 20, 10% DMSO) alone. Animals were topically administered 6 doses. *(P<0.05; t-test)

FIG. 2 shows average clinical scores of animals 24 hours post-challenge in a DTH hypersensitivity reaction study that were treated with either a 5%, 1% or 0.2% solution of ShK-186 or vehicle (9 mM sodium phosphate, 0.72% NaCl, 0.045% polysorbate 20, 10% DMSO) alone. Animals were topically administered 6 doses. *(P<0.05; ANOVA)

FIG. 3A shows average clinical scores of animals 48 hours post-challenge in a DTH hypersensitivity reaction study that were treated with either 5% or 1% solution of ShK-186 or vehicle (9 mM sodium phosphate, 0.72% NaCl, 0.045% polysorbate 20, 10% DMSO) alone. Animals were topically administered 10 doses. *(P<0.05; ANOVA) FIG. 3B shows average clinical scores of animals from 48 hours post-challenge that were treated with a 1% solution of ShK-186 or vehicle alone. Animals were topically administered 10 doses. *(P<0.05; t-test)

FIGS. 4A, 4B, and 4C show the average clinical scores of animals from 3 separate DTH hypersensitivity reaction studies (FIG. 4A, FIG. 4B, FIG. 4C) at 24 hours post-challenge that were treated with either a 5% or 1% solution of ShK-198 or vehicle (9 mM sodium phosphate, 0.72% NaCl, 0.045% polysorbate 20, 10% DMSO) alone. Animals were topically administered 6 doses. *(P<0.05; t-test)

FIGS. 5A and 5B show average clinical scores of animals from 2 separate DTH hypersensitivity studies 24 hours post-challenge that were treated with a 1% or solution of ShK-198 in multiple novel formulations or vehicle alone. Animals were topically administered 6 doses. *(P<0.05; ANOVA) FIG. 5C shows average clinical scores of animals from 48 hours post-challenge that were treated with a 1% or solution of ShK-198 or vehicle alone. Animals were topically administered 10 doses. *(P<0.05; t-test)

FIG. 6 shows average histopathologic rat ear widths (n=5/group) from animals used in a typical delayed-type hypersensitivity study. This graph represents animals that were treated with 5% (50 mg/mL), 1% (10 mg/mL) or 0.2% (2 mg/mL) ShK-186 peptide topical compositions. Vehicle control animals received P6N/10% DMSO alone. One group of animals were included that were sensitized, but not challenged as control for the model. (*P≤0.05 ANOVA to vehicle control; +P≤0.05 student's t-test to vehicle control (no challenge only)).

FIG. 7 shows average inflammation scores from animals used in a typical delayed-type hypersensitivity study (n=5/group). Scoring is based on epidermal and dermal scoring scales as described in EXAMPLE 3. This graph represents animals that were treated with 5% (50 mg/mL), 1% (10 mg/mL) or 0.2% (2 mg/mL) ShK-186 peptide topical compositions. Vehicle control animals received P6N/10% DMSO alone. One group of animals were included that were sensitized, but not challenged as control for the model. (*P≤0.05 ANOVA to vehicle control; +P≤0.05 student's t-test to vehicle control (no challenge only)).

FIG. 8 shows summed histopathology scores from animals used in a typical delayed-type hypersensitivity study (n=5/group). Percent differences from average vehicle control group scores are shown for each group. Scoring is based on the scoring scale provided in EXAMPLE 3. This graph represents animals that were treated with 5% (50 mg/mL), 1% (10 mg/mL) or 0.2% (2 mg/mL) ShK-186 peptide topical compositions. Vehicle control animals received P6N/10% DMSO alone. One group of animals were included that were sensitized, but not challenged as control for the model. (*P≤0.05 ANOVA to vehicle control; +P≤0.05 student's t-test to vehicle control (no challenge only)).

FIG. 9 shows CD8 positive cell counts from animals used in a typical delayed-type hypersensitivity study (n=5/group). Percent differences from average vehicle control group scores are shown for each group. Scoring was carried out as described in EXAMPLE 3. This graph represents animals that were treated with 5% (50 mg/mL), 1% (10 mg/mL) or 0.2% (2 mg/mL) ShK-186 peptide topical compositions. Vehicle control animals received P6N/10% DMSO alone. One group of animals were included that were sensitized, but not challenged as control for the model. (*P≤0.05 ANOVA to vehicle control; +P≤0.05 student's t-test to vehicle control (no challenge only)).

FIGS. 10A, 10B, 10C, 10D, and 10E show photomicrographs of representative ears of each treatment group from animals in a typical DTH study. FIG. 10A shows vehicle control group pinna (H&E, 100×) showing severe swelling and inflammation. FIG. 10B shows no challenge group pinna (H&E, 100×); normal appearance with no indications of inflammation. FIG. 10C shows 5% ShK-186 treatment group pinna (H&E, 100×). FIG. 10D shows 1% ShK-186 treatment group pinna (H&E, 100×). FIG. 10E shows 0.2% Shk-186 treatment group pinna (H&E, 100×).

FIGS. 11A, 11B, 11C, 11D, and 11E show photomicrographs detailing the dark staining of CD8 positive lymphocytes in representative ears of each treatment group from animals in a typical DTH study. FIG. 11A shows vehicle control group pinna (CD8, 100×) showing severe CD8+ cell infiltration (dark stain). FIG. 11B shows no challenge group pinna (CD8, 100×); normal appearance with no indications of CD8+ cell infiltration. FIG. 11C shows 5% ShK-186 treatment group pinna (CD8, 100×). FIG. 11D shows 1% Shk-186 treatment group pinna (CD8, 100×). FIG. 11E shows 0.2% ShK-186 treatment group pinna (CD8, 100).

FIG. 12A shows rat pinna sections from an animal 48 hours post-challenge that was treated with a 1% solution of ShK-186 and stained with antibody from clone P3B3, a ShK specific monoclonal antibody. This animal was topically administered 10 doses. The dark staining shows perfuse and extensive penetration by the ShK-186 peptide. FIG. 12B shows rat pinna from same animal stained with isotype control. Lack of dark staining indicates the P3B3 antibody is specific for the ShK-186 peptide.

FIG. 13A shows rat pinna sections from an animal 48 hours post-challenge that was treated with a 5% solution of ShK-198 and stained with antibody from clone P3B3, a ShK specific monoclonal antibody. This animal was topically administered 10 doses. The dark staining shows perfuse and extensive penetration by the ShK-198 peptide. FIG. 13B shows rat pinna from same animal stained with isotype control. Lack of dark staining indicates the P3B3 antibody is specific for the ShK-198 peptide.

### DETAILED DESCRIPTION

Among the most common T cell mediated inflammatory skin conditions are psoriasis and atopic dermatitis. Psoriasis is a chronic inflammatory skin condition affecting approximately 2-4% of the world's population and is considered the most prevalent immune mediated disease in humans. Atopic dermatitis is a common chronic inflammatory skin disease with a pediatric prevalence of at least 10% in the United States. Atopic dermatitis primarily affects children with disease onset principally occurring between 1 and 5 years of age.

Plaque psoriasis or psoriasis vulgaris is the most common form of psoriasis characterized by erythematous plaques with silvery scales. The major clinical features of atopic dermatitis are itching and pruritis following a chronic or relapsing history and often associated with atopy.

Psoriatic lesions contain a great number of CD45RO+ CLA+ memory T cells that produce the proinflammatory cytokines interferon gamma, interleukin-17 (IL-17), interleukin-22 (IL-22) and tumor necrosis factor alpha, and psoriasis is considered to be primarily a T helper type 17 (Th17) and 22 (Th22) mediated disease. Atopic dermatitis was originally considered a prototypical T helper type 2 (Th2) disease characterized by interleukin(IL)-4, 5 and 13 producing T cells. However in the chronic stages of disease, more interferon gamma production is observed, leading to a mixed T helper type (Th) ½ model for this disease.

Studies of lesional skin from psoriasis and atopic dermatitis have shown that CD4+ T cells from atopic dermatitis are Th2 polarized whereas CD4+ T cell from psoriasis are primarily Th1 and Th17 polarized. CD8+ T cells from both atopic dermatitis and psoriasis are potent producers of proinflammatory cytokines including interferon gamma, IL17 and IL13. Furthermore IL22 is produced by CD4+ and CD8+ T cells in both atopic dermatitis and psoriatic skin. Therefore T cells are important effectors of disease in both atopic dermatitis and psoriasis.

Skin has been estimated to contain more than 20 billion total T cells, more than twice the number present in the blood. Previous studies showed that >95% of T cells in skin are CD45RO+ memory T cells and 80% lack expression of the lymphoid homing receptor CCR7, suggesting that the vast majority of skin-associated T cells are of the effector memory phenotype. Effector memory T cells in skin are primarily nonmigratory, explaining why skin inflammatory lesions that spontaneously resolve (whether from fixed drug eruption or psoriasis) typically recur in the same location. Without regard to their specific cytokine polarization, the majority of T cell-mediated inflammatory skin diseases are caused by long-lived, skin resident effector memory T (T_{EM}) cells.

Upon activation, T_{EM} up-regulate their expression of Kv1.3 K+ ion channels. The T_{EM} that initiate and contribute to damaging autoimmune processes are highly dependent upon these Kv1.3 channels to sustain intracellular calcium levels required for activation, proliferation, and cytokine production. Therefore, the proliferation of T_{EM} is sensitive to Kv1.3 K+ channel blockers. Wulff et al., J. Clin. Invest., 111, 1703-1713 (2003). Other cell types that express the Kv1.3 channel and that are important for inflammation include macrophages, dendritic cells, class-switched memory B-cells, and microglial cells.

Without being bound by theory, it is believed that the toxin-based therapeutic peptides disclosed herein effectively treat psoriasis, atopic dermatitis and other inflammatory skin conditions, including those associated with T_{EM} cells, by blocking Kv1.3 K+ channels. Accordingly, the present disclosure provides methods of using toxin-based peptides in a composition suitable for topical or mucosal administration for treating immune-mediated skin conditions and diseases.

Toxin-Based Peptides/Proteins

Particular examples of toxin-based peptides for use in the methods and topical compositions disclosed herein bind voltage gated channels. Exemplary voltage gated channels include Kv1.1, Kv1.2, Kv1.3, Kv1.4, Kv1.5, Kv1.6, Kv1.7, Kv2.1, Kv3.1, Kv3.2, Kv11.1, Kc1.1, Kc2.1, Kc3.1, Nav1.2, Nav1.4, and Cav1.2.

Toxin peptides are produced by a variety of organisms and have evolved to bind to ion channels and receptors. Native toxin peptides from snakes, scorpions, spiders, bees, snails, and sea anemone are typically 10-80 amino acids in length and contain 2 to 5 disulfide bridges that create compact molecular structures. These peptides appear to have evolved from a small number of structural frameworks. The peptides cluster into families of folding patterns that are conserved through cysteine/disulfide loop structures to maintain a three dimensional structure that contributes to potency, stability, and selectivity (Pennington, et al., Biochemistry, 38, 14549-14558 (1999); Tudor, et al., Eur. J. Biochem., 251, 133-141 (1998); and Jaravine et al., Biochemistry, 36, 1223-1232, (1997)).

As used herein, “toxin-based peptides” or “toxin-based proteins” include a toxin-based peptide of Table 1 (or a variant, D-substituted analog, carboxy-terminal amide, modification, derivative or pharmaceutically acceptable salt thereof) or an ShK-based toxin peptide of Table 2 (or a variant, D-substituted analog, carboxy-terminal amide, modification, derivative or pharmaceutically acceptable salt thereof). Toxin-based peptides can be synthetic or naturally-occurring.

“Toxin-based peptides” or “toxin-based proteins” include any synthetic or naturally-known peptide and those peptide derivatives disclosed in Table 1 as well as variants, D-substituted analogs, carboxy-terminal amides, modifications, derivatives and pharmaceutically acceptable salts thereof. Particular exemplary toxin-based peptides for use in the methods and compositions, disclosed herein include the peptides listed in Table 1, and as shown in the sequence listing as SEQ ID NO:225-256. In various embodiments, a method of treating dermatological conditions includes administering a peptide disclosed herein including a peptide of Table 1 (SEQ ID NO:225-256). In various embodiments, the peptides of Table 1 (SEQ ID NO:225-256) can be used in the production of a topical pharmaceutical composition (or medicament) to treat a dermatological condition.

TABLE 1
Exemplary Toxin-Based Proteins/Peptides

| Sequence/Structure | Shorthand ID | SEQ ID NO: |
|---|---|---|
| LVKCRGTSDCGRPCQQQTGCPNSKCINRMCKCYGC | Pi1 | 225 |
| TISCTNPKQCYPHCKKETGYPNAKCMNRKCKCFGR | Pi2 | 226 |
| TISCTNEKQCYPHCKKETGYPNAKCMNRKCKCFGR | Pi3 | 227 |
| IEAIRCGGSRDCYRPCQKRTGCPNAKCINKTCKCYGCS | Pi4 | 228 |
| ASCRTPKDCADPCRKETGCPYGKCMNRKCKCNRC | HsTx1 | 229 |
| GVPINVSCTGSPQCIKPCKDAGMRFGKCMNRKCHCTPK | AgTx2 | 230 |
| GVPINVKCTGSPQCLKPCKDAGMRFGKCINGKCHCTPK | AgTx1 | 231 |
| GVIINVKCKISRQCLEPCKKAGMRFGKCMNGKCHCTPK | OSK1 | 232 |
| ZKECTGPQHCTNFCRKNKCTHGKCMNRKCKCFNCK | Anuroctoxin | 233 |
| TIINVKCTSPKQCSKPCKELYGSSAGAKCMNGKCKCYNN | NTx | 234 |
| TVIDVKCTSPKQCLPPCKAQFGlRAGAKCMNGKCKCYPH | HgTx1 | 235 |
| QFTNVSCTTSKECWSVCQRLHNTSRGKCMNKKCRCYS | ChTx | 236 |
| VFINAKCRGSPECLPKCKEAIGKAAGKCMNGKCKCYP | Titystoxin-Ka | 237 |
| VCRDWFKETACRHAKSLGNCRTSQKYRANCAKTCELC | BgK | 238 |
| VGINVKCKHSGQCLKPCKDAGMRFGKCINGKCDCTPKG | BmKTx | 239 |
| QFTDVKCTGSKQCWPVCKQMFGKPNGKCMNGKCRCYS | BmTx1 | 240 |
| VFINVKCRGSKECLPACKAAVGKAAGKCMNGKCKCYP | Tc30 | 241 |
| TGPQTTCQAAMCEAGCKGLGKSMESCQGDTCKCKA | Tc32 | 242 |
| AAAISCVGSPECPPKCRAQGCKNGKCMNRKCKCYYC-amide | Vm24 | 243 |
| RTCKDLIPVSECTDIRCRTSMKYRLNLCRKTCGSC | HmK | 244 |
| GCKDNFSANTCKHVKANNNCGSQKYATNCAKTCGKC | Aek | 245 |
| ACKDNFAAATCKHVKENKNCGSQKYATNCAKTCGKC | AsKS | 246 |
| TIINVKCTSPKQCLPPCKAQFGQSAGAKCMNGKCKCYPH | MgTx | 247 |
| GVEINVKCSGSPQCLKPCKDAGMRFGKCMNRKCHCTPK | KTx1 | 248 |
| VRIPVSCKHSGQCLKPCKDAGMRFGKCMNGKCDCTPK | KTx2 | 249 |
| VSCTGSKDCYAPCRKQTGCPNAKCINKSCKCYGC | MTx | 250 |
| QFTDVDCSVSKECWSVCKDLFGVDRGKCMGKKCRCY | IbTx | 251 |
| GVPTDVKCRGSPQCIQPCKDAGMRFGKCMNGKCHCTPK | ODK2 | 252 |
| GVPINVKCRGSPQCIQPCRDAGMRFGKCMNGKCHCTPQ | Bs6 | 253 |
| GVPINVKCRGSRDCLDPCKKAGMRFGKCINSKCHCTP | BoiTx1 | 254 |
| GVPINVPCTGSPQCIKPCKDAGMRFGKCMNRKCHCTPK | AgTx3 | 255 |
| VGIPVSCKHSGQCIKPCKDAGMRFGKCMNRKCDCTPK | KTx3 | 256 |

“ShK” peptides are a subtype of toxin peptides that can also be used in the methods and topical pharmaceutical compositions described herein. ShK peptides were originally isolated from the Caribbean Sea anemone Stichodactyla helianthus. ShK peptides serve as inhibitors of Kv1.3 channels. By inhibiting Kv1.3 channels, ShK can suppress activation, proliferation and/or cytokine production of or by T_{EM}, in certain embodiments, at picomolar concentrations.

As used herein, an “inhibitor” is any peptide as disclosed herein that decreases or eliminates a biological activity that normally results based on the interaction of the peptide with a receptor including biosynthetic and/or catalytic activity, receptor or signal transduction pathway activity, gene transcription or translation, cellular protein transport, and the like.

A native ShK peptide is described in, for example, Pennington, et al., Int. J. Pept. Protein Res., 46, 354-358 (1995). Exemplary ShK structures that are within the scope of the present disclosure are also published in Beeton, et al., Mol. Pharmacol., 67, 1369-1381 (2005); U.S. Publication No. 2008/0221024; PCT Publication No. WO/2012/170392; and in U.S. Pat. Nos. 8,080,523 and 8,440,621.

“ShK-based peptides” or “ShK-based proteins” include any synthetic or naturally-known ShK peptides as well as variants, D-substituted analogs, carboxy-terminal amides, modifications, derivatives and pharmaceutically acceptable salts thereof.

Particular exemplary ShK-based peptides for use with the methods and topical pharmaceutical compositions disclosed herein can include those listed in Table 2, and as shown in the sequence listing as SEQ ID NO:1-224 and SEQ ID NO:257-260. In various embodiments, a method of treating dermatological conditions includes administering a therapeutically-effective amount of a peptide of Table 2 (SEQ ID NO: 1-224 and SEQ ID NO:257-260). In various embodiments, the peptides of Table 2 (SEQ ID NO:1-224 and SEQ ID NO:257-260) can be used in the production of a topical pharmaceutical composition (or medicament) to treat dermatological conditions. Peptides utilized in particular embodiments disclosed herein include those of SEQ ID NO:1, SEQ ID NO:49, SEQ ID NO:208, SEQ ID NO:210, SEQ ID NO:217, SEQ ID NO:218, SEQ ID NO:221, SEQ ID NO:223, and SEQ ID NO:257

TABLE 2
Exemplary ShK-Based Proteins/Peptides

| Sequence/structure | Shorthand ID | SEQ ID NO: |
|---|---|---|
| RSCIDTIPKSRCTAFQCKHSMKYRLSFCRKTCGTC | ShK | 1 |
| RSCIDTIPKSRCTAFQSKHSMKYRLSFCRKTSGTC | ShK-S17/S32 | 2 |
| RSSIDTIPKSRCTAFQCKHSMKYRLSFCRKTCGTS | ShK-S3/S35 | 3 |
| SSCIDTIPKSRCTAFQCKHSMKYRLSFCRKTCGTC | ShK-S1 | 4 |
| (N-acetylR)SCIDTIPKSRCTAFQCKHSMKYRLSFCRKTCGTC | ShK-N-acetylarg 1 | 5 |
| SCIDTIPKSRCTAFQCKHSMKYRLSFCRKTCGTC | ShK-d1 | 6 |
| CIDTIPKSRCTAFQCKHSMKYRLSFCRKTCGTC | ShK-d2 | 7 |
| ASCIDTIPKSRCTAFQCKHSMKYRLSFCRKTCGTC | ShK-A1 | 8 |
| QCIDTIPKSRCTAFQCKHSMKYRLSFCRKTCGTC | ShK-Q2 d1 | 9 |
| ACIDTIPKSRCTAFQCKHSMKYRLSFCRKTCGTC | ShK-A2 d1 | 10 |
| TCIDTIPKSRCTAFQCKHSMKYRLSFCRKTCGTC | ShK-T2 d1 | 11 |
| RQCIDTIPKSRCTAFQCKHSMKYRLSFCRKTCGTC | ShK-Q2 | 12 |
| RACIDTIPKSRCTAFQCKHSMKYRLSFCRKTCGTC | ShK-A2 | 13 |
| RTCIDTIPKSRCTAFQCKHSMKYRLSFCRKTCGTC | ShK-T2 | 14 |
| AQCIDTIPKSRCTAFQCKHSMKYRLSFCRKTCGTC | ShK-Q2 | 15 |
| AACIDTIPKSRCTAFQCKHSMKYRLSFCRKTCGTC | ShK-A1/A2 | 16 |
| ATCIDTIPKSRCTAFQCKHSMKYRLSFCRKTCGTC | ShK-A1/T2 | 17 |
| RSCADTIPKSRCTAFQCKHSMKYRLSFCRKTCGTC | ShK-A1/A4 | 18 |
| RSCADTIPKSRCTAAQCKHSMKYRLSFCRKTCGTC | ShK-A4/A15 | 19 |
| RSCADTIPKSRCTAAQCKHSMKYRASFCRKTCGTC | ShK-A4/A15/A25 | 20 |
| RSCIDAIPKSRCTAFQCKHSMKYRLSFCRKTCGTC | ShK-A6 | 21 |
| RSCIDTIPKSRCTAFQCKHSMKYRLSFCRKTCGTC-amide | ShK-T6 | 22 |
| RSCIDYIPKSRCTAFQCKHSMKYRLSFCRKTCGTC | ShK-Y6 | 23 |
| RSCIDLIPKSRCTAFQCKHSMKYRLSFCRKTCGTC | ShK-L6 | 24 |
| RSCIDTAPKSRCTAFQCKHSMKYRLSFCRKTCGTC | ShK-A7 | 25 |
| RSCADTIPKSRCTAFQCKHSMKYRLSFCRKTCGTC | ShK-A4 | 26 |
| RSCIDTIAKSRCTAFQCKHSMKYRLSFCRKTCGTC | ShK-A8 | 27 |
| RSCIDTIPASRCTAFQCKHSMKYRLSFCRKTCGTC | ShK-A9 | 28 |
| RSCIDTIPESRCTAFQCKHSMKYRLSFCRKTCGTC | ShK-E9 | 29 |
| RSCIDTIPQSRCTAFQCKHSMKYRLSFCRKTCGTC | ShK-Q9 | 30 |
| RSCIDTIPKARCTAFQCKHSMKYRLSFCRKTCGTC | ShK-A10 | 31 |
| RSCIDTIPKSACTAFQCKHSMKYRLSFCRKTCGTC | ShK-A11 | 32 |
| RSCIDTIPKSECTAFQCKHSMKYRLSFCRKTCGTC | ShK-E11 | 33 |
| RSCIDTIPKSQCTAFQCKHSMKYRLSFCRKTCGTC | ShK-Q11 | 34 |
| RSCIDTIPKSRCAAFQCKHSMKYRLSFCRKTCGTC | ShK-A13 | 35 |
| RSCIDTIPKSRCTAAQCKHSMKYRLSFCRKTCGTC | ShK-A15 | 36 |
| RSCIDTIPKSRCTAWQCKHSMKYRLSFCRKTCGTC | ShK-W15 | 37 |
| RSCIDTIPKSRCTA[X(s1)]QCKHSMKYRLSFCRKTCGTC | ShK-X15 | 38 |
| RSCIDTIPKSRCTAAQCKHSMKYRASFCRKTCGTC | ShK-A15/A25 | 39 |
| RSCIDTIPKSRCTAFACKHSMKYRLSFCRKTCGTC | ShK-A16 | 40 |
| RSCIDTIPKSRCTAFECKHSMKYRLSFCRKTCGTC | ShK-E16 | 41 |
| RSCIDTIPKSRCTAFQCAHSMKYRLSFCRKTCGTC | ShK-A18 | 42 |
| RSCIDTIPKSRCTAFQCEHSMKYRLSFCRKTCGTC | ShK-E18 | 43 |
| RSCIDTIPKSRCTAFQCKASMKYRLSFCRKTCGTC | ShK-A19 | 44 |
| RSCIDTIPKSRCTAFQCKKSMKYRLSFCRKTCGTC | ShK-K19 | 45 |
| RSCIDTIPKSRCTAFQCKHAMKYRLSFCRKTCGTC | ShK-A20 | 46 |
| RSCIDTIPKSRCTAFQCKHSAKYRLSFCRKTCGTC | ShK-A21 | 47 |
| RSCIDTIPKSRCTAFQCKHS[X(s2)]KYRLSFCRKTCGTC | ShK-X21 | 48 |
| RSCIDTIPKSRCTAFQCKHS(Nle)KYRLSFCRKTCGTC | ShK-Nle21 | 49 |
| RSCIDTIPKSRCTAFQCKHSMAYRLSFCRKTCGTC | ShK-A22 | 50 |
| RSCIDTIPKSRCTAFQCKHSMEYRLSFCRKTCGTC | ShK-E22 | 51 |
| RSCIDTIPKSRCTAFQCKHSMRYRLSFCRKTCGTC | ShK-R22 | 52 |
| RSCIDTIPKSRCTAFQCKHSM[X(s3)]YRLSFCRKTCGTC | ShK-X22 | 53 |
| RSCIDTIPKSRCTAFQCKHSM(Nle)YRLSFCRKTCGTC | ShK-Nle22 | 54 |
| RSCIDTIPKSRCTAFQCKHSM(Orn)YRLSFCRKTCGTC | ShK-Orn22 | 55 |
| RSCIDTIPKSRCTAFQCKHSM(Homocit)YRLSFCRKTCGTC | ShK-Homocit22 | 56 |
| RSCIDTIPKSRCTAFQCKHSM(Dap)YRLSFCRKTCGTC | ShK-diamino-propionic22 | 57 |
| RSCIDTIPKSRCTAFQCKHSMKARLSFCRKTCGTC | ShK-A23 | 58 |
| RSCIDTIPKSRCTAFQCKHSMKSRLSFCRKTCGTC | ShK-S23 | 59 |
| RSCIDTIPKSRCTAFQCKHSMKFRLSFCRKTCGTC | ShK-F23 | 60 |
| RSCIDTIPKSRCTAFQCKHSMK[X(s4)]RLSFCRKTCGTC | ShK-X23 | 61 |
| RSCIDTIPKSRCTAFQCKHSMK(NitroF)RLSFCRKTCGTC | ShK-Nitrophe23 | 62 |
| RSCIDTIPKSRCTAFQCKHSMK(AminoF)RLSFCRKTCGTCC | ShK-Aminophe23 | 63 |
| RSCIDTIPKSRCTAFQCKHSMK(BenzylF)RLSFCRKTCGTC | ShK-Benzylphe23 | 64 |
| RSCIDTIPKSRCTAFQCKHSMKYALSFCRKTCGTC | ShK-A24 | 65 |
| RSCIDTIPKSRCTAFQCKHSMKYELSFCRKTCGTC | ShK-E24 | 66 |
| RSCIDTIPKSRCTAFQCKHSMKYRASFCRKTCGTC | ShK-A25 | 67 |
| RSCIDTIPKSRCTAFQCKHSMKYRLAFCRKTCGTC | ShK-A26 | 68 |
| RSCIDTIPKSRCTAFQCKHSMKYRLSACRKTCGTC | ShK-A27 | 69 |
| RSCIDTIPKSRCTAFQCKHSMKYRLS[X(s27)]CRKTCGTC | ShK-X27 | 70 |
| RSCIDTIPKSRCTAFQCKHSMKYRLSFCAKTCGTC | ShK-A29 | 71 |
| RSCIDTIPKSRCTAFQCKHSMKYRLSFCRATCGTC | ShK-A30 | 72 |
| RSCIDTIPKSRCTAFQCKHSMKYRLSFCRKACGTC | ShK-A31 | 73 |
| RSCIDTIPKSRCTAFQCKHSMKYRLSFCRKTCGAC | ShK-A34 | 74 |
| SCADTIPKSRCTAFQCKHSMKYRLSFCRKTCGTC | ShK-A4d1 | 75 |
| SCADTIPKSRCTAAQCKHSMKYRLSFCRKTCGTC | ShK-A4/A15d1 | 76 |
| SCADTIPKSRCTAAQCKHSMKYRASFCRKTCGTC | ShK-A4/A15/A25d1 | 77 |
| SCIDAIPKSRCTAFQCKHSMKYRLSFCRKTCGTC | ShK-A6d1 | 78 |
| SCIDTAPKSRCTAFQCKHSMKYRLSFCRKTCGTC | ShK-A7d1 | 79 |
| SCIDTIAKSRCTAFQCKHSMKYRLSFCRKTCGTC | ShK-A8d1 | 80 |
| SCIDTIPASRCTAFQCKHSMKYRLSFCRKTCGTC | ShK-A9d1 | 81 |
| SCIDTIPESRCTAFQCKHSMKYRLSFCRKTCGTC | ShK-E9d1 | 82 |
| SCIDTIPQSRCTAFQCKHSMKYRLSFCRKTCGTC | ShK-Q9d1 | 83 |
| SCIDTIPKARCTAFQCKHSMKYRLSFCRKTCGTC | ShK-A10d1 | 84 |
| SCIDTIPKSACTAFQCKHSMKYRLSFCRKTCGTC | ShK-A11d1 | 85 |
| SCIDTIPKSECTAFQCKHSMKYRLSFCRKTCGTC | ShK-E11d1 | 86 |
| SCIDTIPKSQCTAFQCKHSMKYRLSFCRKTCGTC | ShK-Q11d1 | 87 |
| SCIDTIPKSRCAAFQCKHSMKYRLSFCRKTCGTC | ShK-A13d1 | 88 |
| SCIDTIPKSRCTAAQCKHSMKYRLSFCRKTCGTC | ShK-A15d1 | 89 |
| SCIDTIPKSRCTAWQCKHSMKYRLSFCRKTCGTC | ShK-W15d1 | 90 |
| SCIDTIPKSRCTA[X(s15)]QCKHSMKYRLSFCRKTCGTC | ShK-X15d1 | 91 |
| SCIDTIPKSRCTAAQCKHSMKYRASFCRKTCGTC | ShK-A15/A25d1 | 92 |
| SCIDTIPKSRCTAFACKHSMKYRLSFCRKTCGTC | ShK-A16d1 | 93 |
| SCIDTIPKSRCTAFECKHSMKYRLSFCRKTCGTC | ShK-E16d1 | 94 |
| SCIDTIPKSRCTAFQCAHSMKYRLSFCRKTCGTC | ShK-A18d1 | 95 |
| SCIDTIPKSRCTAFQCEHSMKYRLSFCRKTCGTC | ShK-E18d1 | 96 |
| SCIDTIPKSRCTAFQCKASMKYRLSFCRKTCGTC | ShK-A19d1 | 97 |
| SCIDTIPKSRCTAFQCKKSMKYRLSFCRKTCGTC | ShK-K19d1 | 98 |
| SCIDTIPKSRCTAFQCKHAMKYRLSFCRKTCGTC | ShK-A20d1 | 99 |
| SCIDTIPKSRCTAFQCKHSAKYRLSFCRKTCGTC | ShK-A21d1 | 100 |
| SCIDTIPKSRCTAFQCKHS[X(s2)]KYRLSFCRKTCGTC | ShK-X21d1 | 101 |
| SCIDTIPKSRCTAFQCKHS(Nle)KYRLSFCRKTCGTC | ShK-Nle21d1 | 102 |
| SCIDTIPKSRCTAFQCKHSMAYRLSFCRKTCGTC | ShK-A22d1 | 103 |
| SCIDTIPKSRCTAFQCKHSMEYRLSFCRKTCGTC | ShK-E22d1 | 104 |
| SCIDTIPKSRCTAFQCKHSMRYRLSFCRKTCGTC | ShK-R22d1 | 105 |
| SCIDTIPKSRCTAFQCKHSM[X(s3)]YRLSFCRKTCGTC | ShK-X22d1 | 106 |
| SCIDTIPKSRCTAFQCKHSM(Nle)YRLSFCRKTCGTC | ShK-Nle22d1 | 107 |
| SCIDTIPKSRCTAFQCKHSM(Orn)YRLSFCRKTCGTC | ShK-Orn22d1 | 108 |
| SCIDTIPKSRCTAFQCKHSM(Homocit)YRLSFCRKTCGTC | ShK-Homocit22d1 | 109 |
| SCIDTIPKSRCTAFQCKHSM(Dap)YRLSFCRKTCGTC | ShK-Dap22d1 | 110 |
| SCIDTIPKSRCTAFQCKHSMKARLSFCRKTCGTC | ShK-A23d1 | 111 |
| SCIDTIPKSRCTAFQCKHSMKSRLSFCRKTCGTC | ShK-S23d1 | 112 |
| SCIDTIPKSRCTAFQCKHSMKFRLSFCRKTCGTC | ShK-F23d1 | 113 |
| SCIDTIPKSRCTAFQCKHSMK[X(s4)]RLSFCRKTCGTC | ShK-X23d1 | 114 |
| SCIDTIPKSRCTAFQCKHSMK(NitroF)RLSFCRKTCGTC | ShK-Nitrophe23d1 | 115 |
| SCIDTIPKSRCTAFQCKHSMK(AminoF)RLSFCRKTCGTC | ShK-Aminophe23d1 | 116 |
| SCIDTIPKSRCTAFQCKHSMK(BenzylF)RLSFCRKTCGTShK- | Benzylphe23d1 | 117 |
| SCIDTIPKSRCTAFQCKHSMKYALSFCRKTCGTC | ShK-A24d1 | 118 |
| SCIDTIPKSRCTAFQCKHSMKYELSFCRKTCGTC | ShK-E24d1 | 119 |
| SCIDTIPKSRCTAFQCKHSMKYRASFCRKTCGTC | ShK-A25d1 | 120 |
| SCIDTIPKSRCTAFQCKHSMKYRLAFCRKTCGTC | ShK-A26d1 | 121 |
| SCIDTIPKSRCTAFQCKHSMKYRLSACRKTCGTC | ShK-A27d1 | 122 |
| SCIDTIPKSRCTAFQCKHSMKYRLS[X(s5)]CRKTCGTC | ShK-X27d1 | 123 |
| SCIDTIPKSRCTAFQCKHSMKYRLSFCAKTCGTC | ShK-A29d1 | 124 |
| SCIDTIPKSRCTAFQCKHSMKYRLSFCRATCGTC | ShK-A30d1 | 125 |
| SCIDTIPKSRCTAFQCKHSMKYRLSFCRKACGTC | ShK-A31d1 | 126 |
| SCIDTIPKSRCTAFQCKHSMKYRLSFCRKTCGAC | ShK-A34d1 | 127 |
| YSCIDTIPKSRCTAFQCKHSMKYRLSFCRKTCGTC | ShK-Y1 | 128 |
| KSCIDTIPKSRCTAFQCKHSMKYRLSFCRKTCGTC | ShK-K1 | 129 |
| HSCIDTIPKSRCTAFQCKHSMKYRLSFCRKTCGTC | ShK-H1 | 130 |
| QSCIDTIPKSRCTAFQCKHSMKYRLSFCRKTCGTC | ShK-Q1 | 131 |
| PPRSCIDTIPKSRCTAFQCKHSMKYRLSFCRKTCGTC | PP-ShK | 132 |
| MRSCIDTIPKSRCTAFQCKHSMKYRLSFCRKTCGTC | M-ShK | 133 |
| GRSCIDTIPKSRCTAFQCKHSMKYRLSFCRKTCGTC | G-ShK | 134 |
| YSCIDTIPKSRCTAFQCKHSMAYRLSFCRKTCGTC | ShK-Y1/A22 | 135 |
| KSCIDTIPKSRCTAFQCKHSMAYRLSFCRKTCGTC | ShK-K1/A22 | 136 |
| HSCIDTIPKSRCTAFQCKHSMAYRLSFCRKTCGTC | ShK-H1/A22 | 137 |
| QSCIDTIPKSRCTAFQCKHSMAYRLSFCRKTCGTC | ShK-Q1/A22 | 138 |
| PPRSCIDTIPKSRCTAFQCKHSMAYRLSFCRKTCGTC | PP- ShK-A22 | 139 |
| MRSCIDTIPKSRCTAFQCKHSMAYRLSFCRKTCGTC | M-ShK-A22 | 140 |
| GRSCIDTIPKSRCTAFQCKHSMAYRLSFCRKTCGTC | G- ShK-A22 | 141 |
| RSCIDTIPASRCTAFQCKHSMAYRLSFCRKTCGTC | ShK-A9/A22 | 142 |
| SCIDTIPASRCTAFQCKHSMAYRLSFCRKTCGTC | ShK-A9/A22d1 | 143 |
| RSCIDTIPVSRCTAFQCKHSMKYRLSFCRKTCGTC | ShK-V9 | 144 |
| RSCIDTIPVSRCTAFQCKHSMAYRLSFCRKTCGTC | ShK-V9/A22 | 145 |
| SCIDTIPVSRCTAFQCKHSMKYRLSFCRKTCGTC | ShK-V9d1 | 146 |
| SCIDTIPVSRCTAFQCKHSMAYRLSFCRKTCGTC | ShK-V9/A22d1 | 147 |
| RSCIDTIPESRCTAFQCKHSMAYRLSFCRKTCGTC | ShK-E9/A22 | 148 |
| SCIDTIPESRCTAFQCKHSMAYRLSFCRKTCGTC | ShK-E9/A22d1 | 149 |
| RSCIDTIPKSACTAFQCKHSMAYRLSFCRKTCGTC | ShK-A11/A22 | 150 |
| SCIDTIPKSACTAFQCKHSMAYRLSFCRKTCGTC | ShK-A11/A22d1 | 151 |
| RSCIDTIPKSECTAFQCKHSMAYRLSFCRKTCGTC | ShK-E11/A22 | 152 |
| SCIDTIPKSECTAFQCKHSMAYRLSFCRKTCGTC | ShK-E11/A22d1 | 153 |
| RSCIDTIPKSRCTDFQCKHSMKYRLSFCRKTCGTC | ShK-D14 | 154 |
| RSCIDTIPKSRCTDFQCKHSMAYRLSFCRKTCGTC | ShK-D14/A22 | 155 |
| SCIDTIPKSRCTDFQCKHSMKYRLSFCRKTCGTC | ShK-D14d1 | 156 |
| SCIDTIPKSRCTDFQCKHSMAYRLSFCRKTCGTC | ShK-D14/A22d1 | 157 |
| RSCIDTIPKSRCTAAQCKHSMAYRLSFCRKTCGTC | ShK-A15/A22 | 158 |
| SCIDTIPKSRCTAAQCKHSMAYRLSFCRKTCGTC | ShK-A15/A22d1 | 159 |
| RSCIDTIPKSRCTAIQCKHSMKYRLSFCRKTCGTC | ShK-I15 | 160 |
| RSCIDTIPKSRCTAIQCKHSMAYRLSFCRKTCGTC | ShK-I15/A22 | 161 |
| SCIDTIPKSRCTAIQCKHSMKYRLSFCRKTCGTC | ShK-I15d1 | 162 |
| SCIDTIPKSRCTAIQCKHSMAYRLSFCRKTCGTC | ShK-I15/A22d1 | 163 |
| RSCIDTIPKSRCTAVQCKHSMKYRLSFCRKTCGTC | ShK-V15 | 164 |
| RSCIDTIPKSRCTAVQCKHSMAYRLSFCRKTCGTC | ShK-V15/A22 | 165 |
| SCIDTIPKSRCTAVQCKHSMKYRLSFCRKTCGTC | ShK-V15d1 | 166 |
| SCIDTIPKSRCTAVQCKHSMAYRLSFCRKTCGTC | ShK-V15/A22d1 | 167 |
| RSCIDTIPKSRCTAFRCKHSMKYRLSFCRKTCGTC | ShK-R16 | 168 |
| RSCIDTIPKSRCTAFRCKHSMAYRLSFCRKTCGTC | ShK-R16/A22 | 169 |
| SCIDTIPKSRCTAFRCKHSMKYRLSFCRKTCGTC | ShK-R16d1 | 170 |
| SCIDTIPKSRCTAFRCKHSMAYRLSFCRKTCGTC | ShK-R16/A22d1 | 171 |
| RSCIDTIPKSRCTAFKCKHSMKYRLSFCRKTCGTC | ShK-K16 | 172 |
| RSCIDTIPKSRCTAFKCKHSMAYRLSFCRKTCGTC | ShK-K16/A22 | 173 |
| SCIDTIPKSRCTAFKCKHSMKYRLSFCRKTCGTC | ShK-K16d1 | 174 |
| SCIDTIPKSRCTAFKCKHSMAYRLSFCRKTCGTC | ShK-K16/A22d1 | 175 |
| RSCIDTIPASECTAFQCKHSMKYRLSFCRKTCGTC | ShK-A9/E11 | 176 |
| RSCIDTIPASECTAFQCKHSMAYRLSFCRKTCGTC | ShK-A9/E11/A22 | 177 |
| SCIDTIPASECTAFQCKHSMKYRLSFCRKTCGTC | ShK-A9/E11d1 | 178 |
| SCIDTIPASECTAFQCKHSMAYRLSFCRKTCGTC | ShK-A9/E11/A22d1 | 179 |
| RSCIDTIPVSECTAFQCKHSMKYRLSFCRKTCGTC | ShK-V9/E11 | 180 |
| RSCIDTIPVSECTAFQCKHSMAYRLSFCRKTCGTC | ShK-V9/E11/A22 | 181 |
| SCIDTIPVSECTAFQCKHSMKYRLSFCRKTCGTC | ShK-V9/E11d1 | 182 |
| SCIDTIPVSECTAFQCKHSMAYRLSFCRKTCGTC | ShK-V9/E11/A22d1 | 183 |
| RSCIDTIPVSACTAFQCKHSMKYRLSFCRKTCGTC | ShK-V9/A11 | 184 |
| RSCIDTIPVSACTAFQCKHSMAYRLSFCRKTCGTC | ShK-V9/A11/A22 | 185 |
| SCIDTIPVSACTAFQCKHSMKYRLSFCRKTCGTC | ShK-V9/Al1dl | 186 |
| SCIDTIPVSACTAFQCKHSMAYRLSFCRKTCGTC | ShK-V9/A11/A22d1 | 187 |
| RSCIDTIPASACTAFQCKHSMKYRLSFCRKTCGTC | ShK-A9/A11 | 188 |
| RSCIDTIPASACTAFQCKHSMAYRLSFCRKTCGTC | ShK-A9/A11/A22 | 189 |
| SCIDTIPASACTAFQCKHSMKYRLSFCRKTCGTC | ShK-A9/A11d1 | 190 |
| SCIDTIPASACTAFQCKHSMAYRLSFCRKTCGTC | ShK-A9/A11/A22d1 | 191 |
| RSCIDTIPKSECTDIRCKHSMKYRLSFCRKTCGTC | ShK-E11/D14/115/R16 | 192 |
| RSCIDTIPKSECTDIRCKHSMAYRLSFCRKTCGTC | ShK-E11/D14/115/R16/A22 | 193 |
| SCIDTIPKSECTDIRCKHSMKYRLSFCRKTCGTC | ShK-E11/D14/115/R16d1 | 194 |
| SCIDTIPKSECTDIRCKHSMAYRLSFCRKTCGTC | ShK-E11/D14/115/R16/A22d1 | 195 |
| RSCIDTIPVSECTDIRCKHSMKYRLSFCRKTCGTC | ShK-V9/E11/D14/115/R16 | 196 |
| RSCIDTIPVSECTDIRCKHSMAYRLSFCRKTCGTC | ShK-V9/E11/D14/115/R16/A22 | 197 |
| SCIDTIPVSECTDIRCKHSMKYRLSFCRKTCGTC | ShK-V9/E11/D14/115/R16d1 | 198 |
| SCIDTIPVSECTDIRCKHSMAYRLSFCRKTCGTC | ShK-V9/E11/D14/115/R16/A22d1 | 199 |
| RSCIDTIPVSECTDIQCKHSMKYRLSFCRKTCGTC | ShK-V9/E11/D14415 | 200 |
| RSCIDTIPVSECTDIQCKHSMAYRLSFCRKTCGTC | ShK-V9/E11/D14/115/A22 | 201 |
| SCIDTIPVSECTDIQCKHSMKYRLSFCRKTCGTC | ShK-V9/E11/D14/115d1 | 202 |
| SCIDTIPVSECTDIQCKHSMAYRLSFCRKTCGTC | ShK-V9/E11/D14/115/A22d1 | 203 |
| RTCKDLIPVSECTDIRCKHSMKYRLSFCRKTCGTC | ShK-T2/K4/L6/V9/E11/ | 204 |
| | D14/115/R16 | |
| RTCKDLIPVSECTDIRCKHSMAYRLSFCRKTCGTC | ShK-T2/K4/L6/V9/E11/ | 205 |
| | D14/115/R16/A22 | |
| TCKDLIPVSECTDIRCKHSMKYRLSFCRKTCGTC | ShK-T2/K4/L6N9/E11/ | 206 |
| | D14/115/R16d1 | |
| TCKDLIPVSECTDIRCKHSMAYRLSFCRKTCGTC | ShK-T2/K4/L6N9/E11/ | 207 |
| | D14/115/R16/A22d1 | |
| (L-PhosphoTyr)-AEEAc- | ShK(L5) | 208 |
| RSCIDTIPKSRCTAFQCKHSMKYRLSFCRKTCGTC | | |
| (L-Tyr)-AEEAc- | ShK(L4) | 209 |
| RSCIDTIPKSRCTAFQCKHSMKYRLSFCRKTCGTC | | |
| (L-Tyr)-AEEAc- | ShK-198 | 210 |
| RSCIDTIPKSRCTAFQCKHSMKYRLSFCRKTCGTC-amide | | |
| QSCADTIPKSRCTAAQCKHSMKYRLSFCRKTCGTC | ShK-Q1/A4/A15 | 211 |
| QSCADTIPKSRCTAAQCKHSMAYRLSFCRKTCGTC | ShK-Q1/A4/A15/A22 | 212 |
| QSCADTIPKSRCTAAQCKHSM(Dap)YRLSFCRKTCGTC | ShK-Q1/A4/A15/Dap22 | 213 |
| QSCADTIPKSRCTAAQCKHSMKYRASFCRKTCGTC | ShK-Q1/A4/A15/A25 | 214 |
| QSCADTIPKSRCTAAQCKHSMAYRASFCRKTCGTC | ShK-Q1/A4/A15/A22/A25 | 215 |
| QSCADTIPKSRCTAAQCKHSM(Dap)YRASFCRKTCGTC | ShK-Q1/A4/A15/Dap22/A25 | 216 |
| (L-PhosphoTyr)-AEEAc- | ShK-186 | 217 |
| RSCIDTIPKSRCTAFQCKHSMKYRLSFCRKTCGTC-amide | | |
| (Para-phosphono-Phe)-AEEAc- | ShK-192 | 218 |
| RSCIDTIPKSRCTAFQCKHS(Nle)KYRLSFCRKTCGTC-amide | | |
| (Phosphonomethyl-Phe)-AEEAc- | ShK-191 | 219 |
| RSCIDTIPKSRCTAFQCKHSMKYRLSFCRKTCGTC-amide | | |
| (Phosphonomethyl-Phe)-AEEAc- | ShK-191/Nle21 | 220 |
| RSCIDTIPKSRCTAFQCKHS(Nle)KYRLSFCRKTCGTC-amide | | |
| DOTA-aminohexanoicacid-(L-Tyr)-AEEAc- | ShK-221 | 221 |
| RSCIDTIPKSRCTAFQCKHSMKYRLSFCRKTCGTC-amide | | |
| (Para-phosphono-Phe)-AEEAc- | ShK-223 | 222 |
| RSCIDTIPKSRCTAFKCKHS(Nle)KYRLSFCRKTCGTC-amide | | |
| (Para-phosphono-Phe)-AEEAc- | ShK-190 | 223 |
| RSCIDTIPKSRCTAFQCKHSMKYRLSFCRKTCGTC-amide | | |
| RSCIDTIPKSRCTAFQCKHS(Nle)(Dap)YRLSFCRKTCGTC | | 224 |
| (L-PhosphoTyr)-AEEAc- | | 257 |
| RSCIDTIPKSRCTAFQCKHS(Nle)KYRLSFCRKTCGTC | | |
| (L-Tyr)-AEEAc- | | 258 |
| RSCIDTIPKSRCTAFQCKHS(Nle)KYRLSFCRKTCGTC | | |
| (L-PhosphoTyr)-AEEAc- | | 259 |
| RSCIDTIPKSRCTAFQCKHS(Nle)KYRLSFCRKTCGTC-amide | | |
| (L-Tyr)-AEEAc- | | 260 |
| RSCIDTIPKSRCTAFQCKHS(Nle)KYRLSFCRKTCGTC-amide | | |

Notes:
X(s1), X(s2), X(s3), etc. each refer independently to nonfunctional amino acid residues.
N-acetylR refers to N-acetyl larginine
Nle refers to Norleucine
Orn refers to Ornithine
Homocit refers to Homocitrulline
NitroF refers to Nitrophenylalanine
AminoF refers to Aminophenylalanine
BenzylF refers to Benzylphenylalanine
AEEAc refers to Aminoethyloxyethyloxy acetic acid
Dap refers to Diaminopropionic acid
DOTA refers to 1,4,7,10-tetraacyclododecane-1,4,7,10-tetraacetic acid

Those skilled in the art are aware of techniques for designing proteins as disclosed herein with enhanced properties, such as alanine scanning, rational design based on alignment mediated mutagenesis using known sequences, and/or molecular modeling. For example, proteins as disclosed herein can be designed to remove protease cleavage sites (e.g., trypsin cleavage sites at K or R residues and/or chymotrypsin cleavage sites at F, Y, or W residues). Nonhydrolyzable phosphate substitutions also impart a stabilizing effect on the phosphate groups, as well as stability against phosphatase enzymes. Nonhydrolyzable phosphate groups include phosphonate analogs of phosphotyrosine such as 4-phosphonomethylphenylalanine (Pmp) 4-phosphonodifluoromethyl phenylalanine (F2Pmp), paraphosphonophenylalanine, monofluorophosphonomethyl phenylalanine, sulfono(difluormethyl)phenylalanine (F2Smp) and hydroxylphosphono methylphenylalanine. In other embodiments, phosphotyrosine mimetics can be used such as the nonhydrolyzable phosphotyrosine mimetic L-O-(2-malonyl)tyrosine (OMT), the phosphotyrosyl mimetic fluoro-O-malonyl tyrosine (FOMT), and other analogs that utilize carboxylic acid groups to replicate phosphate functionality as described in Burke and Lee, Acc. Chem. Res., 36, 426-433 (2003). In a further embodiment, nonhydrolyzable analogs include methyl-, aryloxy-, and thio-ethyl phosphonic acids. In a still further embodiment, nonhydrolyzable phosphate derivatives include difluoromethylenephosphonic and difluoromethylenesulfonic acid.

To improve the pharmacokinetic and pharmacodynamic (PK/PD) properties of the structure of the proteins disclosed herein, residues that are sensitive to degradation properties can be substituted, replaced, or modified. Modification of the C-terminal acid function with an amide can also impart stability. These changes to the primary structure of toxin-based therapeutic proteins can be combined with an anionic moiety at the N-terminus to produce a stable and selective Kv1.3 blocker. In order to produce a peptide/protein with a higher half-life in vivo, variants or modifications of the peptides/proteins can be prepared wherein key proteolytic digestion sites can be substituted to reduce protease susceptibility. This can include substitution of nonessential residues with conservative isosteric replacements (e.g., Lys to Lys (acetyl) or Gln) and or neutral replacements (Ala).

“Variants” of peptides/proteins disclosed herein include peptides/proteins having one or more amino acid additions, deletions, stop positions, or substitutions, as compared to a peptide/protein disclosed herein.

An amino acid substitution can be a conservative or a non-conservative substitution. Variants of toxin-based therapeutic proteins disclosed herein can include those having one or more conservative amino acid substitutions. A “conservative substitution” involves a substitution found in one of the following conservative substitutions groups: Group 1: Alanine (Ala; A), Glycine (Gly; G), Serine (Ser; S), Threonine (Thr; T); Group 2: Aspartic acid (Asp; D), Glutamic acid (Glu; E); Group 3: Asparagine (Asn; N), Glutamine (Gln; Q); Group 4: Arginine (Arg; R), Lysine (Lys; K), Histidine (His; H); Group 5: Isoleucine (Ile; I), Leucine (Leu; L), Methionine (Met; M), Valine (Val; V); and Group 6: Phenylalanine (Phe; F), Tyrosine (Tyr; Y), Tryptophan (Trp; W).

Additionally, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and lie. Other groups including amino acids that are considered conservative substitutions for one another include: sulfur-containing: Met and Cys; acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W.H. Freeman and Company.

Variants of peptides/proteins disclosed herein also include peptides/proteins with at least 70% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to a peptide/protein sequence disclosed herein.

Variants of peptides/proteins for use in the topical compositions and formulations disclosed herein based on the peptides/proteins include peptides/proteins that share: 70% sequence identity with any of SEQ ID NO:225-256; 75% sequence identity with any of SEQ ID NO:225-256; 80% sequence identity with any of SEQ ID NO:225-256; 81% sequence identity with any of SEQ ID NO:225-256; 82% sequence identity with any of SEQ ID NO:225-256; 83% sequence identity with any of SEQ ID NO:225-256; 84% sequence identity with any of SEQ ID NO:225-256; 85% sequence identity with any of SEQ ID NO:225-256; 86% sequence identity with any of SEQ ID NO: 225-256; 87% sequence identity with any of SEQ ID NO:225-256; 88% sequence identity with any of SEQ ID NO:225-256; 89% sequence identity with any of SEQ ID NO:225-256; 90% sequence identity with any of SEQ ID NO:225-256; 91% sequence identity with any of SEQ ID NO:225-256; 92% sequence identity with any of SEQ ID NO:225-256; 93% sequence identity with any of SEQ ID NO:225-256; 94% sequence identity with any of SEQ ID NO:225-256; 95% sequence identity with any of SEQ ID NO:225-256; 96% sequence identity with any of SEQ ID NO:225-256; 97% sequence identity with any of SEQ ID NO:225-256; 98% sequence identity with any of SEQ ID NO:225-256; or 99% sequence identity with any of SEQ ID NO:225-256.

Variants of the peptides/proteins for use in the topical compositions and formulations disclosed herein based on ShK-based proteins include proteins that share: 80% sequence identity with any of SEQ ID NO:1-224 and/or SEQ ID NO:257-260; 81% sequence identity with any of SEQ ID NO:1-224 and/or SEQ ID NO:257-260; 82% sequence identity with any of SEQ ID NO:1-224 and/or SEQ ID NO:257-260; 83% sequence identity with any of SEQ ID NO:1-224 and/or SEQ ID NO:257-260; 84% sequence identity with any of SEQ ID NO:1-224 and/or SEQ ID NO:257-260; 85% sequence identity with any of SEQ ID NO:1-224 and/or SEQ ID NO:257-260; 86% sequence identity with any of SEQ ID NO:1-224 and/or SEQ ID NO:257-260; 87% sequence identity with any of SEQ ID NO:1-224 and/or SEQ ID NO:257-260; 88% sequence identity with any of SEQ ID NO:1-224 and/or SEQ ID NO:257-260; 89% sequence identity with any of SEQ ID NO:1-224 and/or SEQ ID NO:257-260; 90% sequence identity with any of SEQ ID NO:1-224 and/or SEQ ID NO:257-260; 91% sequence identity with any of SEQ ID NO:1-224 and/or SEQ ID NO:257-260; 92% sequence identity with any of SEQ ID NO:1-224 and/or SEQ ID NO:257-260; 93% sequence identity with any of SEQ ID NO:1-224 and/or SEQ ID NO:257-260; 94% sequence identity with any of SEQ ID NO:1-224 and/or SEQ ID NO:257-260; 95% sequence identity with any of SEQ ID NO:1-224 and/or SEQ ID NO:257-260; 96% sequence identity with any of SEQ ID NO:1-224 and/or SEQ ID NO:257-260; 97% sequence identity with any of SEQ ID NO:1-224 and/or SEQ ID NO:257-260; 98% sequence identity with any of SEQ ID NO:1-224 and/or SEQ ID NO:257-260; or 99% sequence identity with any of SEQ ID NO: 1-224 and/or SEQ ID NO:257-260.

Particular exemplary embodiments include peptides/proteins wherein the peptides/proteins share 80% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity, or 99% sequence identity with SEQ ID NO:208. In another embodiment, variants include peptides/proteins sharing 80% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity, or 99% sequence identity with SEQ ID NO:209. In another embodiment, variants include peptides/proteins sharing 80% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity, or 99% sequence identity with SEQ ID NO:217. In another embodiment, variants include peptides/proteins sharing 80% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity, or 99% sequence identity, with SEQ ID NO:210. In another embodiment, variants include peptides/proteins sharing 80% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity, or 99% sequence identity with SEQ ID NO:218. In another embodiment, variants include peptides/proteins sharing 80% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity, or 99% sequence identity with SEQ ID NO:208. In another embodiment, variants include peptides/proteins sharing 80% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity, or 99% sequence identity with SEQ ID NO:257.

“% sequence identity” refers to a relationship between two or more sequences, as determined by comparing the sequences. In the art, “identity” also means the degree of sequence relatedness between peptide/protein sequences as determined by the match between strings of such amino acid sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine sequence identity are designed to give the best match between the sequences tested. Methods to determine sequence identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” mean any set of values or parameters which originally load with the software when first initialized.

“D-substituted analogs” include peptides/proteins disclosed herein having one more L-amino acids substituted with D-amino acids. The D-amino acid can be the same amino acid type as that found in the peptide/protein sequence or can be a different amino acid. Accordingly, D-analogs are also variants.

“Modifications” include peptides/proteins disclosed herein, wherein one or more amino acids have been replaced with a non-amino acid component, or where the amino acid has been conjugated to a functional group or a functional group has been otherwise associated with an amino acid or protein. The modified amino acid can be, e.g., a glycosylated amino acid, a PEGylated amino acid, a farnesylated amino acid, an acetylated amino acid, a biotinylated amino acid, an amino acid conjugated to a lipid moiety, an amino acid conjugated to human serum albumin, or an amino acid conjugated to an organic derivatizing agent. The presence of modified amino acids can be advantageous in, for example, (a) increasing protein serum or tissue half-life and/or functional in vivo half-life, (b) reducing protein antigenicity, (c) increasing protein storage stability, (d) increasing protein solubility, (e) prolonging circulating or tissue resident time, (f) increasing bioavailability, e.g. increasing the area under the curve (AUCsc) and/or (g) increasing skin penetration. Amino acid(s) can be modified, for example, co-translationally or post-translationally during recombinant production (e.g., N-linked glycosylation at N-X-S/T motifs during expression in mammalian cells) or modified by synthetic means. The modified amino acid can be within the sequence or at the terminal end of a sequence. Modifications can include derivatives as described elsewhere herein.

The C-terminus can be a carboxylic acid or an amide group, preferably a carboxylic acid group for each of the toxin-based therapeutic proteins. The present disclosure also relates to the peptides/proteins further modified by (i) additions made to the C-terminus, such as Tyr, iodo-Tyr, a fluorescent tag, or (ii) additions made to the N-terminus, such as Tyr, iodo-Tyr, pyroglutamate, or a fluorescent tag.

In addition, residues or groups of residues known to the skilled artisan to improve stability can be added to the C-terminus and/or N-terminus.

In particular embodiments, the C-terminus is an acid (for example, COOH) or an amide (for example, CONH2). “Amide” refers to NH₂, in particular embodiments, attached to the C-terminal end of a protein. In various embodiments, the C-terminal hydroxyl group (OH) of an acid is substituted with an amide. Such substitution is designated herein using the term “amide” or as the C-terminal amino acid-NH₂, as in “-Cys-NH₂.”

The safety, potency, and specificity of a variety of peptides/proteins have been investigated, and attaching the peptide/protein to an organic or inorganic chemical entity that has an anionic charge has been shown to improve the suitability for use in a topical pharmaceutical composition or formulation. The site of attachment can be the N-terminus, but modifications are not limited to attachment at this site.

Examples of appropriate chemical entities include L-Pmp(OH₂); D-Pmp(OH₂); D-Pmp(OHEt); Pmp(Et2); D-Pmp(Et2); L-Tyr; L-Tyr(PO₃H₂) (p-phospho-Tyrosine); L-Phe(p-NH₂); L-Phe(p-CO₂H); L-Aspartate; D-Aspartate; L-Glutamate; and D-Glutamate. The abbreviations used are defined as follows: Pmp (p-phosphonomethyl-phenylalanine); and Ppa (p-phosphatityl-phenylalanine). Alternatives to PmP and Ppa include Pfp (p-Phosphono(difluoro-methyl)-Phenylalanine) and Pkp (p-Phosphono-methylketo-Phenylalanine).

Exemplary chemical entities can be attached by way of a linker, such as an aminoethyloxyethyloxy-acetyl acid linker (referred to herein as AEEAc), or by any other suitable means. Examples of chemical entity/linker combinations include AEEAc-L-Pmp(OH₂); AEEAc-D-Pmp(OH₂); AEEAc-D-Pmp(OHEt); AEEAc-L-Pmp(Et2); AEEAc-D-Pmp(Et2); AEEAc-L-Tyr; AEEAc-L-Tyr(PO₃H₂); AEEAc-L-Phe(p-NH₂); AEEAc-L-Phe(p-CO₂H); AEEAc-L-Aspartate; AEEAc-D-Aspartate; AEEAc-L-Glutamate; and AEEAc-D-Glutamate. In the chemical entities generally, where the amino acid residue has a chiral center, the D and/or L enantiomer of the amino acid residue can be used.

The peptides/proteins disclosed herein can be modified by the N-terminal attachment of AEEAc and/or an amide attachment at the C-terminal (for example, ShK-186 (SEQ ID NO:217) and ShK-192 (SEQ ID NO:218)). AEEAc can interchangeably refer to aminoethyloxyethyloxyacetic acid and Fmoc-aminoethyloxyethyloxyacetic acid when being used to describe the linker during the formation process. When being used to refer to the linker in specific peptides/proteins in their final state, the term refers to aminoethyloxyethyloxyacetic acid.

The peptides/proteins disclosed herein can be modified by the addition of polyethylene glycol (PEG), human serum albumin, antibodies, fatty acids, antibody fragments including the Fab and Fc regions, hydroxyethyl starch, dextran, oligosaccharides, polysialic acids, hyaluronic acid, dextrin, poly(2-ethyl 2-oxazolone), polyglutamic acid (PGA), N-(2-hydroxypropyl)methacrylamide copolymer (HPMA), unstructured hydrophilic sequences of amino acids including in particular the amino acids Ala, Glu, Gly, Ser, and Thr, and many other linkers and additions as described in Schmidt, S. R. (ed.), Fusion Protein Targeting for Biopharmaceuticals: Applications and Challenges, John Wiley and Sons: Hoboken N.J., 2013. PEG groups can be attached to c amino groups of lysine using: (a) PEG succinimidyl carbonate, (b) PEG benzotriazole carbonate, (c) PEG dichlorotriazine, (d) PEG tresylate, (e) PEG p-nitrophenyl carbonate, (f) PEG trichlorophenyl carbonate, (g) PEG carbonylimidazole, and (h) PEG succinimidyl succinate. PEG groups can be attached to cysteines by degradable linkers including para- or ortho-disulfide of benzyl urethane. Site specific introduction of PEG can be achieved by reductive alkylation with PEG-aldehyde or by glyceraldehyde modification of alpha-amino groups in the presence of sodium cyanoborohydride. PEGylation chemistries have been described in numerous publications including Robert, et al., Advanced Drug Delivery Reviews, 54, 459-476 (2002). Oligosaccharides can be N-linked or O-linked. N-linked oligosaccharides, including polysialic acid are added by the producing cell line by attachment to the consensus sequence of Asn-Xaa-Ser/Thr where Xaa is anything but proline. O-linked oligosaccharides are attached to Ser or Thr.

Particular embodiments include peptides/proteins of SEQ ID NO:1-260 to which an organic or inorganic chemical entity that has an anionic charge is attached via AEEAc.

Another example of a peptide/protein is an ShK-based DOTA-conjugate of ShK-186 (referred to as ShK-221; SEQ ID NO:221). “DOTA” refers to 1, 4, 7, 10-tetraazacyclododecane-1, 4, 7, 10-tetraacetic acid which can be attached to the N-terminus of the therapeutic proteins disclosed herein via aminohexanoic acid. DOTA conjugation provides a site for chelating metal atoms such as Indium or Gadolinium. Other molecules that can be conjugated to peptides/proteins disclosed herein include diethylene triamine pentaacetic acid (DTPA), Nitrilotriacetic acid (NTA), Ethylenediaminetetraacetic acid (EDTA), Iminodiacetic acid (IDA), ethylene glycol tetraacetic acid (EGTA), 1,2-bis(o-aminophenoxy)ethane-N,N,N′,N′-tetraacetic acid (BAPTA), 1,4,7-triazacyclononane-N,N′,N″-triacetic acid (NOTA), and related molecules.

The present disclosure is further directed to derivatives of the disclosed peptides/proteins. “Derivatives” include peptides/proteins having acylic permutations in which the cyclic permutants retain the native bridging pattern of the native peptide/protein. In one embodiment, the cyclized peptide/protein includes a linear peptide/protein and a peptide/protein linker, wherein the N- and C-termini of the linear peptide/protein are linked via the peptide/protein linker to form the amide cyclized peptide/protein backbone. In some embodiments, the peptide/protein linker includes amino acids selected from Gly, Ala, and combinations thereof.

Various cyclization methods can be applied to the peptides/proteins described herein. The peptides/proteins described herein can be readily cyclized using BOC-chemistry to introduce Ala, Gly, or Ala/Gly bridges, as well as combinations thereof or other residues as described by Schnolzer, et al., Int J Pept Protein Res., 40, 180-193 (1992). Cyclizing peptides/proteins can improve their stability and reduce the susceptibility to proteolysis, without affecting the affinity of the peptides/proteins for their specific targets.

Each peptide/protein disclosed herein can also include additions, deletions, stop positions, substitutions, replacements, conjugations, associations, or permutations at any position including positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of a peptide/protein sequence disclosed herein. Accordingly, in particular embodiments each amino acid position of each peptide/protein can be an Xaa position wherein Xaa denotes an addition, deletion, stop position, substitution, replacement, conjugation, association, or permutation of the amino acid at the particular position. In particular embodiments, each toxin-based therapeutic protein has 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Xaa positions at one or more of positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60.

A peptide/protein as disclosed herein can have more than one change (addition, deletion, stop position, substitution, replacement, conjugation, association, or permutation) and qualify as one or more of a variant, D-substituted analog, carboxy-terminal amide, modification, and/or derivative. That is, inclusion of one classification of variant, D-substituted analog, carboxy-terminal amide, modification, and/or derivative is not exclusive to inclusion in other classifications and all are collectively referred to as “peptides/proteins” herein. One example includes SEQ ID NO: 1 wherein the amino acid at position 21 is norleucine (Nle) and/or the amino acid at position 22 is replaced with diaminopropionic acid (Dap).

In any of the peptides/proteins where position 21 is a Met, the Met can be substituted to impart a stabilizing effect against oxidation. In one embodiment, a Met at position 21 is substituted with Nle. In any of SEQ ID NO:1-260, having a Met at position 21, this Met can be substituted with Nle. In any of SEQ ID NO:1-260, having a Lys at position 22, this Lys can be substituted with diaminopropionic acid (Dap). Accordingly, one embodiment disclosed herein includes SEQ ID NO:1 wherein the Met at position 21 is substituted with Nle, an amide is present at the C-terminus and/or an anionic moiety is present at the N-terminus.

“Nonfunctional amino acid residue” refers to amino acid residues in D- or L-form having sidechains that lack acidic, basic, or aromatic groups. Exemplary nonfunctional amino acid residues include Met, Gly, Ala, Val, Ile, Leu, and Nle.

In particular embodiments disclosed herein, the peptide/protein has at least 20 amino acids, at least 21 amino acids, at least 22 amino acids, at least 23 amino acids, at least 24 amino acids, at least 25 amino acids, at least 26 amino acids, at least 27 amino acids, at least 28 amino acids, at least 29 amino acids, at least 30 amino acids, at least 31 amino acids, at least 32 amino acids, at least 33 amino acids, at least 34 amino acids, at least 35 amino acids, at least 36 amino acids, at least 37 amino acids, at least 38 amino acids, at least 39 amino acids, at least 40 amino acids, at least 41 amino acids, at least 42 amino acids, at least 43 amino acids, at least 44 amino acids, at least 45 amino acids, at least 46 amino acids, at least 47 amino acids, at least 48 amino acids, at least 49 amino acids, at least 50 amino acids, at least 51 amino acids, at least 52 amino acids, at least 53 amino acids, at least 54 amino acids, at least 55 amino acids, at least 56 amino acids, at least 57 amino acids, at least 58 amino acids, at least 59 amino acids, at least 60 amino acids, at least 61 amino acids, at least 62 amino acids, at least 63 amino acids, at least 64 amino acids, at least 65 amino acids, at least 66 amino acids, at least 67 amino acids, at least 68 amino acids, at least 69 amino acids, at least 70 amino acids, at least 71 amino acids, at least 72 amino acids, at least 73 amino acids, at least 74 amino acids, at least 75 amino acids, at least 76 amino acids, at least 77 amino acids, at least 78 amino acids, at least 79 amino acids, or at least 80 amino acids.

In additional embodiments, the peptide/protein has 20 amino acids, 21 amino acids, 22 amino acids, 23 amino acids, 24 amino acids, 25 amino acids, 26 amino acids, 27 amino acids, 28 amino acids, 29 amino acids, 30 amino acids, 31 amino acids, 32 amino acids, 33 amino acids, 34 amino acids, 35 amino acids, 36 amino acids, 37 amino acids, 38 amino acids, 39 amino acids, 40 amino acids, 41 amino acids, 42 amino acids, 43 amino acids, 44 amino acids, 45 amino acids, 46 amino acids, 47 amino acids, 48 amino acids, 49 amino acids, 50 amino acids, 51 amino acids, 52 amino acids, 53 amino acids, 54 amino acids, 55 amino acids, 56 amino acids, 57 amino acids, 58 amino acids, 59 amino acids, 60 amino acids, 61 amino acids, 62 amino acids, 63 amino acids, 64 amino acids, 65 amino acids, 66 amino acids, 67 amino acids, 68 amino acids, 69 amino acids, 70 amino acids, 71 amino acids, 72 amino acids, 73 amino acids, 74 amino acids, 75 amino acids, 76 amino acids, 77 amino acids, 78 amino acids, 79 amino acids, or 80 amino acids.

In additional embodiments disclosed herein the peptide/protein has at least one disulfide bridge, at least two disulfide bridges, at least three disulfide bridges, at least four disulfide bridges, or at least five disulfide bridges.

In additional embodiments, the peptide/protein has one disulfidebridge, two disulfide bridges, three disulfide bridges, four disulfide bridges, or five disulfide bridges.

Peptides/proteins also suitable for topical pharmaceutical compositions, formulations and uses disclosed herein include those having a molecular weight between 500 and 50,000 Daltons.

Particularly relevant peptides/proteins include those that act upon cation channels such as Na⁺, K⁺, or Ca²⁺ channels, anion channels such as Cl⁻ channels or ligand-gated channels such as nicotinic acetyl choline receptors (NAChRs). These channels include both ligand and voltage-gated ion channels that are present extracellularly and/or intracellularly. Extracellular channels or receptors include kanate; α-amino-3-hydroxy-5-methyl-4-isoxazole propionic acid (AMPA); N-methyl-D-aspartate (NMDA) and acetylcholine receptors (such as α9/α10 subtype (nAChR)); serotonin (5-hydroxytryptamine, 5-HT) receptors; and glycine and γ-butyric (GABA) receptors. Intracellular receptors can include cyclic AMP (cAMP), cyclic GMP (cGMP), Ca, and G-protein receptors.

Particular examples of peptides/proteins useful for the topical applications disclosed herein include toxin proteins, including ShK proteins, that target voltage gated channels. Exemplary voltage gated channels include Kv1.1, Kv1.2, Kv1.3, Kv1.4, Kv1.5, Kv1.6, Kv1.7, Kv2.1, Kv3.1, Kv3.2, Kv11.1, Kc1.1, Kc2.1, Kc3.1, Nav1.2, Nav1.4, and Cav1.2 channels.

Prodrugs of the peptides/proteins described herein can also be used. The term “prodrug” refers to a peptide/protein that can undergo biotransformation (e.g., either spontaneous or enzymatic) within a subject to release, or to convert (e.g., enzymatically, mechanically, electromagnetically, etc.) an active or more active form of the peptide/protein. Prodrugs can be used to overcome issues associated with stability, toxicity, lack of specificity, or limited bioavailability. Exemplary prodrugs include an active peptide/protein and a chemical masking group (e.g., a group that reversibly suppresses the activity of the peptide/protein). Some preferred prodrugs are variants or modifications of peptides/proteins that have sequences that are cleavable under metabolic conditions. Exemplary prodrugs become active or more active in vivo or in vitro when they undergo a biochemical transformation (e.g., phosphorylation, hydrogenation, dehydrogenation, glycosylation, etc.). Prodrugs often offer advantages of solubility, tissue compatibility, or delayed release (See e.g., Bundgard, Design of Prodrugs, pp. 7-9, 21-24, Elsevier, Amsterdam (1985); and Silverman, The Organic Chemistry of Drug Design and Drug Action, pp. 352-401, Academic Press, San Diego, Calif. (1992)).

Topical Formulations of the Disclosed Peptides/Proteins

Topical formulations or compositions of the disclosure are composed of a peptide/protein as disclosed herein and a dermatological base. The dermatological base can be composed of hydrophilic, hydrophobic, emulsifying or gelling agents, a penetration enhancing agent, a preservative and/or an antioxidant. Exemplary dermatological base ingredients include water, ethanol, 2-propanol, glycerol, propylene glycol, sorbitol, macrogol, dimethyl sulfoxide, acetone, petrolatum, hard paraffin, soft and liquid paraffin, triglycerides, wax, liquid wax ester, a partial glyceride, a silicon oil, an anionic surfactant, a zwitterionic surfactant, a nonionic surfactant, bentonite, carbomer, carmellose sodium, hydroxyethyl cellulose, hydroxypropyl cellulose, hypromellose, alkyl-4-hydroxybenzoates, sorbic acid, benzyl alcohol, phenylethyl alcohol, butylhydroxyanisol, butylated hydroxytoluene, tocopherol, tocopherol acetate, ascorbyl palmitate, and/or a combination of any one or more of the above.

The main ingredient in a hydrophilic preparation is water and water soluble alcohols, although polyvalent alcohols such as propylene glycol can be added to enhance the moistening properties of the preparation.

Hydrophobic bases are typically defined by their spreadability and ease of skin absorption. Highly spreadable hydrophobic bases are preferred as they do not leave a greasy feeling on the skin. Exemplary embodiments of easily spreadable hydrophobic bases include yellow wax, fluid wax esters (isopropyl myristate and ethylhexyl palmitate) and silicone oils that contain polyorganoziloxane as the functional group such as dimethicone, phenyl methyl polysiloxane and cyclomethicone.

For dermatological bases that contain both hydrophilic and hydrophobic regents, emulsifying agents can be used. Emulsifiers can include but are not limited to nonionic, anionic, cationic and/or zwitterionic emulsifiers. Exemplary anionic emulsifiers include but are not limited to sodium stearate, aluminum stearate, sodium dodecyl sulfate, sodium cetyl stearyl sulfate, sodium lauryl ether sulfate, and/or sodium dioctyl sulfosuccinate. Exemplary cationic emulsifiers include but are not limited to cetyl trimethyl ammonium bromide, benzalkonium bromide and/or cetylpyrdinium chloride. Exemplary zwitterionic emulsifiers include but are not limited to phosphatidylcholine, betaine monohydrate, and/or Dehyton K®. Exemplary nonionic emulsifiers include but are not limited to PEG-30 stearate, glycerol monostearate, glycerol monooleate, glycerol monoisostearate, partial glyceride medium chain, Tween®, polyoxyethylene sorbitan fatty acid esters, polyoxyethylene (20) sorbitan monostearate (polysorbate 20), sorbitan laurate, sorbitan monooleate, sorbitan monopalmitate, sorbitan tristearate, sorbitan sesquiloleate, saccharose monostearate, saccharose cocoate, cetomagrogol 1000, macrogol cetostearyl ether, macrogol oleyl ether, lauromacrogol 400, cholesterol, wool fat, acetylated wool fat, hydrated wool fat, wool fat alcohols, macrogol 1000 glycerol monooleate, macrogol 1000 glycerol monostearate, macrogol 300 glycerol tris hydroxystearate, macrogol 5 glycerol stearate, macrogol glycerol hydroxystearate, and/or triglycerol diisostearate.

Exemplary penetration enhancing agents include but are not limited to a sulfoxide, such as dimethyl sulfoxide; an azone, such as lauropsam; a pyrrodlidone, such as 2-pyrrolidone; an alcohol or an alkanol, such as ethanol or decanol; a glycol, such as propylene glycol; a surfactant, and a terpene.

Exemplary gelling agents include but are not limited to synthetic polyacrylic acid (carbomer), semi-synthetic cellulose derivatives such as sodium carboxymethylcellulose and hydroxyethyl cellulose, xanthan, inorganic bentonite, and hypromellose.

Exemplary preservative include but are not limited to the alkyl-4-hydroxybenzoates (methyl, ethyl, propyl, butyl, parabens), sorbic acid, benzoic acid, benzyl alcohol, phenoxyethanol, and phenyl ethyl alcohol. Ethanol, 2-propanol and propylene glycol at high concentrations can also have a preservative effect.

Exemplary antioxidants include but are not limited to alpha-tocopherol, ethyl gallate, propyl gallate, octyl gallate, lauryl gallate, decyl ester gallate, butylhydroxyanisol, butylated hydroxytoluene and the ascorbic acid esters including but not limited to myristin, palmitin and stearin acid esters.

In particular embodiments disclosed herein, propylene glycol can be used at at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, and at least 95% in topical formulations of the peptide/protein.

In particular embodiments disclosed herein, dimethyl sulfoxide can be used at at least 5%, at least 10%, at least 15%, at least 20%, at least 25%, at least 30%, at least 35%, at least 40%, at least 45%, at least 50%, at least 55%, and at least 60% in topical formulations of the peptide/protein.

In particular embodiments disclosed herein, acetone can be used at between 0.1 and 1%, at least 1%, at least 2%, at least 3%, at least 4%, at least 5%, at least 6%, at least 7%, at least 8%, at least 9%, at least 10%, at least 1%, at least 12%, at least 13%, at least 14%, at least 15%, at least 20% in topical formulations of the peptide/protein.

In particular embodiments disclosed herein, petrolatum can be used at at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, and at least 95% in topical formulations of the peptide/protein.

In particular embodiments disclosed herein, paraffins can be used at at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, and at least 95% in topical formulations of the peptides/proteins.

In particular embodiments disclosed herein, mono- di- and triglycerides, including but not limited to medium chain triglycerides and caprylic, capric, stearic and succinic triglyceride, can be used at at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, and at least 95% in topical formulations of the peptide/protein.

In particular embodiments disclosed herein, wax including but not limited to beeswax, cetyl esters wax and microcrystalline wax can be used at at least 1%, at least 5%, at least 10%, at least 15%, at least 20%, at least 25%, at least 30%, at least 35%, at least 40%, at least 45%, and at least 50% in topical formulations of the peptide/protein.

In particular embodiments disclosed herein, bentonite can be used at at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, and at least 80% in topical formulations of the peptide/protein.

In particular embodiments disclosed herein, carbomers can be used at 0.01-0.1%, 0.1-1%, 1-2%, 2-3%, 3-4%, 4-5%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, and at least 95% in topical formulations of the peptide/protein.

In particular embodiments disclosed herein, hydroxyethyl cellulose can be used at 0.01-0.1%, 0.1-1%, 1-2%, 2-3%, 3-4%, 4-5%, at least 5%, at least 10%, at least 15%, and at least 20% in topical formulations of the peptide/protein.

In particular embodiments disclosed herein, hydroxypropyl cellulose can be used at 0.01-0.1%, 0.1-1%, 1-2%, 2-3%, 3-4%, 4-5%, at least 5%, at least 10%, at least 15%, and at least 20% in topical formulations of the peptide/protein.

In particular embodiments disclosed herein, hypromellose can be used at 0.01-0.1%, 0.1-1%, 1-2%, 2-3%, 3-4%, 4-5%, at least 5%, at least 10%, at least 15%, and at least 20% in topical formulations of the peptide/protein.

In particular embodiments disclosed herein, sorbic acid can be used at 0.01-0.1%, 0.1-1%, 1-2%, 2-3%, 3-4%, 4-5%, and at least 5% in topical formulations of the peptide/protein.

In particular embodiments disclosed herein, benzyl alcohol can be used at 0.01-0.1%, 0.1-1%, 1-2%, 2-3%, 3-4%, 4-5%, at least 5%, at least 10%, at least 15%, at least 20%, at least 25%, at least 30%, at least 35%, at least 40%, at least 45%, and at least 50% in topical formulations of the peptide/protein.

In particular embodiments disclosed herein, butylated hydroxytoluene can be used at 0.01-0.1%, 0.1-1%, 1-2%, 2-3%, 3-4%, 4-5%, and at least 5% in topical formulations of the peptide/protein.

In particular embodiments disclosed herein, tocopherol and alpha tocopherol can be used at 0.001-0.01%, 0.01-0.1%, 0.1-1%, and at least 1% in topical formulations of the peptide/protein.

In particular embodiments disclosed herein, ascorbyl palmitate can be used at 0.001-0.01%, 0.01-0.1%, 0.1-1%, and at least 1% in topical formulations of the peptide/protein.

In particular embodiments disclosed herein, isopropyl miyristate can be used at 0.01-0.1%, 0.1-1%, 1-2%, 2-3%, 3-4%, 4-5%, 5-10%, 10-20%, 20-30%, 30-40%, 40-50% and 50-60% in topical formulations of the peptide/protein.

In particular embodiments disclosed herein, the palmitates, including but not limited to ascorbyl palmitate, cetyl palmitate, glyceryl palmitate, isopropyl palmitate, and sorbitan monopalmitate, can be used at 0.0001-0.001%, 0.001%-0.01%, 0.01-0.1%, 0.1-1%, 1-2%, 2-3%, 3-4%, 4-5%, 5-100%, 10-20%, 20-30%, 30-40%, and 40-50% in topical formulations of the peptide/protein.

In particular embodiments disclosed herein, dimethicone, phenyl methyl polysiloxane and cyclomethicone can be used at 0.1-1%, 1-2%, 2-3%, 3-4%, 4-5%, 5-10%, and 10-20% in topical formulations of the peptide/protein.

In particular embodiments disclosed herein, aluminum stearate can be used at 0.001%-0.01%, 0.01-0.1%, 0.1-1%, 1-2%, 2-3%, 3-4%, and 4-5% in topical formulations of the peptide/protein.

In particular embodiments disclosed herein, sodium cetostearyl sulfate can be used at 0.01-0.1%, 0.1-1%, 1-2%, 2-3%, 3-4%, and 4-5% in topical formulations of the peptide/protein.

In particular embodiments disclosed herein, disodium laureth sulfosuccinate can be used at 0.01-0.1%, 0.1-1%, 1-2%, 2-3%, 3-4%, 4-5%, 5-10%, 10-15% and 15-20% in topical formulations of the peptide/protein.

In particular embodiments disclosed herein, benzalkonium bromide can be used at 0.01-0.1%, 0.1-1%, 1-2%, 2-3%, 3-4%, and 4-5% in topical formulations of the peptide/protein.

In particular embodiments disclosed herein, the betaines including but not limited to betaine monohydrate and Dehyton K®, can be used at 0.01-0.1%, 0.1-1%, 1-2%, 2-3%, 3-4%, 4-5% and 5-10% in topical formulations of the peptide/protein.

In particular embodiments disclosed herein glycerol stearate, glycerol monostearate, sorbitan monostearate, glycerol monooleate, isopropyl isostearate, methyl stearate, polyethylene glycol stearate, pegoxol stearate, polyoxyl stearate, polyoxyl glycerol stearate, sorbitan monolaurate, sorbitan isostearate, sorbitan monooleate, sorbitan monostearate, sorbitan monopalmitate, sorbitan sesquioleate, sorbitan trioleate, saccharose monostearate, saccharose cocoate, cetostearyl alcohol, polyoxyl 20 cetostearyl ether, sodium cetostearyl sulfate, cetomacrogol 400, macrogol oleyl ether, cetomagrogol 1000, macrogol cetostearyl ether, ethylhexyl hydroxystearate, hydroxyoctacosanyl hydroxystearate, and propylene glycol monopalmitostearate can be used at 0.01-0.1%, 0.1-1%, 1-2%, 2-3%, 3-4%, 4-5% and 5-10%, 10-15%, 15-20%, and 20-30% in topical formulations of the peptide/protein.

In particular embodiments disclosed herein, polyoxyethylene fatty acid esters can be used at 0.1-1%, 1-2%, 2-3%, 3-4%, 4-5% and 5-10% in topical formulations of the peptide/protein.

In particular embodiments disclosed herein, cholesterol can be used at 0.1-1%, 1-2%, 2-3%, 3-4%, 4-5% and 5-10% in topical formulations of the peptide/protein.

In particular embodiments disclosed herein, lanolin and its derivatives, including but not limited to lanolin alcohol, anhydrous lanolin, ethoxylated lanolin, hydrogenated lanolin, polyoxyl lanolin and PEG-75 lanolin, can be used at 0.1-1%, 1-2%, 2-3%, 3-4%, 4-5%, 5-10%, 10-15%, 15-20%, 20-30%, 30-40% and 40-50% in topical formulations of the peptide/protein.

In particular embodiments disclosed herein, polyethylene glycol fatty acid derivatives including but not limited to PEG-100 stearate, PEG 6-32 stearate, PEG-12 glycerol laurate, PEG-120 glyceryl stearate, PEG-2 stearate, PEG-25 propylene glycol stearate, PEG-4 dilaurate, PEG-4 laurate, PEG-5 oleate, PEG-50 stearate, PEG-54 hydrogenated castor oil, PEG-60 hydrogenated castor oil and PEG-7 methyl ether, can be used at 0.1-1%, 1-2%, 2-3%, 3-4%, 4-5%, 5-10%, 10-15%, 15-20%, 20-30%, and 30-40% in topical formulations of the peptide/protein.

In particular embodiments disclosed herein, the carbomers, including but not limited to Carbomer-1342, -1382, -934, -934P, -940, -941, and carbomer homopolymer type B, can be used at 0.01-0.1%, 0.1-1%, 1-2%, 2-3%, 3-4%, 4-5% and 5-10% in topical formulations of the peptide/protein.

In particular embodiments disclosed herein, xanthan can be used at 0.01-0.1%, 0.1-1%, and 1-2% in topical formulations of the peptide/protein.

In particular embodiments disclosed herein, hypromellose and trihydroxystearin can be used at 0.1-1%, 1-2%, 2-3% and 3-4% in topical formulations of the peptide/protein.

In particular embodiments disclosed herein, sodium benzoate, denatonium benzoate, benzoic acid, sorbic acid, benzyl alcohol, phenoxyethanol and phenylethyl alcohol can be used at 0.01-0.1%, 0.1-1%, 1-2% and 2-3% in topical formulations of the peptide/protein.

In particular embodiments disclosed herein, ethyl gallate, propyl gallate, octyl gallate, lauryl gallate, and decyl ester gallate can be used at 0.01-0.1%, 0.1-1%, 1-2% and 2-3% in topical formulations of the peptide/protein.

In particular embodiments disclosed herein, butylhydroxyanisol and butylated hydroxytoluene can be used at 0.0001-0.001%, 0.001%-0.01%, 0.01-0.1%, 0.1-1%, 1-2% and 2-3% in topical formulations of the peptide/protein.

The formulations of the present disclosure can also include emulsions that contain both an oil and water phase. The emulsion can be a simple emulsion (oil-in-water, water-in-oil) or a complex emulsion (for example oil-in-water-in-oil). The formulations are not limited by the type and complexity of the emulsion preparation.

The formulations of the present disclosure can also include suspensions, including but not limited to hydrophilic and lipophilic suspensions. The formulation is not limited by the type and complexity of the suspension preparation.

The formulations of the present disclosure include but are not limited to semisolid preparations that include hydrophobic, water emulsifying, and hydrophilic ointments. The formulation is not limited as to the type or composition of the ointment.

Exemplary hydrophobic ointments can use as a base petrolatum, liquid paraffins, vegetable oils, animal fats, synthetic glycerides, waxes, and/or liquid polyalkylsiloxanes.

Exemplary water emulsifying ointments can include water-in-oil emulsifying agents such as for example lanolin, lanolin alcohols, sorbitan esters, monoglycerides and fatty alcohols or oil-in-water emulsifying agents such as sulfated fatty alcohols, polysorbates, macrogol cetostearyl ether or esters of fatty acids with macrogols.

Exemplary hydrophilic ointments have bases that are miscible in water including for example the polyethylene glycols.

The formulations of the present disclosure can also include creams including but not limited to lipophilic, hydrophilic and amphiphilic creams. The formulations are not limited as to the type or composition of the cream.

Exemplary lipophilic creams contain the typical components of hydrophilic ointments in addition to water-in-oil emulsifiers as described elsewhere in this application. The external phase is lipophilic.

Exemplary hydrophilic creams have an aqueous external phase and contain oil-in-water emulsifiers as described elsewhere in this application. Hydrophilic creams can contain a mixture of emulsifying agents including but not limited to both hydrophilic and lipophilic emulsifiers described elsewhere in this disclosure.

Exemplary amphiphilic creams have a continuous aqueous and lipophilic phase and can be diluted with both water and lipids.

The topical formulations of the present disclosure can also include hydrogels, pastes and powders. The formulations are not limited as to the type or composition of the hydrogels, pastes and powders.

Topical compositions include a defined amount of a peptide/protein including one or more of SEQ ID NO:1-224 and SEQ ID NO:257-260 and SEQ ID NO:225-256 that are added to the formulations of the invention to make a topical drug product or topical composition for the treatment of autoimmune or inflammatory diseases of the skin and/or mucosa. The concentration of the peptide/protein in the final topical composition can be 0.0001%, 0.001%, 0.01%, 0.1%, 1%, 2%, 3%, 4%, 5%, 6%, 7%, 8%, 9%, 10%, 11%, 12%, 13%, 14%, or 15%. The concentration of the peptide/protein in the final topical composition can be from 0.0001-0.001%, 0.001-0.01%, 0.01-0.1%, 0.1-1%, 1-2%, 2-3%, 3-4%, 4-5%, 5-10%, 10-15% or 15-20%.

The topical compositions of the present disclosure can include more than one peptide/proteins disclosed herein or the peptide/protein in combination with another anti-inflammatory product, such as for example a topical steroid.

Topical compositions include a peptide/protein as disclosed herein and at least one pharmaceutically acceptable carrier. Pharmaceutically acceptable carriers include those that do not produce significantly adverse, allergic, or other untoward reactions that outweigh the benefit of administration, whether for research, prophylactic, and/or therapeutic treatments. Exemplary pharmaceutically acceptable carriers and associated formulations are disclosed in Troy, D. B. and Beringer, P. (eds.) Remington: The Science and Practice of Pharmacy, Lippincott; Philadelphia, 2006. 21st Edition. Pharmaceutical compositions are prepared to meet sterility, pyrogenicity, and/or general safety and purity standards as required by U.S. Food and Drug Administration (FDA) Office of Biological Standards and/or other relevant foreign regulatory agencies.

Exemplary generally used pharmaceutically acceptable carriers include any and all absorption delaying agents, antioxidants, binders, buffering agents, bulking agents, chelating agents, co-solvents, coatings, coloring agents, disintegration agents, dispersion media, emulsifiers, fillers, flavoring agents, gels, isotonic agents, lubricants, perfuming agents, preservatives, releasing agents, salts, solvents, stabilizers, sweetening agents, surfactants, wetting agents, and the like.

Exemplary buffering agents include citrate buffers, succinate buffers, tartrate buffers, fumarate buffers, gluconate buffers, oxalate buffers, lactate buffers, acetate buffers, phosphate buffers, histidine buffers, and trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens, methyl paraben, propyl paraben, catechol, resorcinol, cyclohexanol, and 3-pentanol.

More particular examples of preservatives include benzalkonium chloride (≤0.025%), sorbic acid, benzethonium chloride (≤0.01%), chlorobutanol (≤0.5%), phenylmercuric acetate (≤0.004%), phenylmercuric nitrate (≤0.004%), thimerosal (≤0.01%), methylparaben (0.1-0.2%) and propylparabens (≤0.04%). Other preservatives that can be used include mercury derivatives, alcohols, parabens, quarternary ammonium compounds, polyquarternium compounds, and chlorhexidine. Inclusion of preservatives is especially beneficial to prevent contamination (e.g., bacterial contamination) when topical compositions are packaged in a multi-dose container.

Exemplary isotonic agents include polyhydric sugar alcohols, trihydric sugar alcohols, or higher sugar alcohols, such as glycerin, erythritol, arabitol, xylitol, sorbitol, and/or mannitol.

Exemplary stabilizers include organic sugars, polyhydric sugar alcohols, polyethylene glycol, sulfur-containing reducing agents, amino acids, low molecular weight polypeptides, proteins, immunoglobulins, hydrophilic polymers, and/or polysaccharides.

Exemplary antioxidants include alpha-tocopherol, ascorbic acid, ascorbyl palmitate, butylated hydroxyanisole (BHA), butylated hydroxytoluene (BHT), citric acid, cysteine hydrochloride, ethylenediamine tetraacetic acid (EDTA), lecithin, metal chelating agents, methionine, oil soluble antioxidants, phosphoric acid, propyl gallate, sodium bisulfite, sodium metabisulfite, sodium sulfite, sorbitol, tartaric acid, and/or vitamin E. More particular examples/amounts of antioxidants include ethylene diaminetetraacetic acid (EDTA) (≤0.1%), sodium bisulfite (≤0.1%), sodium metabisulfite (≤0.1%), and thiourea (≤0.1%).

Exemplary pharmaceutically acceptable salts include inorganic and organic addition salts, such as acetates, benzoates, citrates, fumarates, hydrochloride, isothionates, maleates, methane-sulfonates, nitrates, phosphates, propionates, salicylates, succinates, sulphates, tartrates, theophylline acetates, and/or trifluoroacetates. Lower alkyl quaternary ammonium salts can also be used.

Solubilizers can be used in topical compositions, including Poloxamer-407, Puronic® F-68, Pluronic® F-127, polysorbates, polyethylene-35-castor oil, hydroxypropyl-beta-cyclodextrin, methyl-beta cyclodextrin, n-octenyl succinate starch, other cyclodextrins, tyloxapol, alpha-tocopherol polyethylene glycol succinate, medium chain triglycerides, sesame oil, arachis oil, safflower oil, mustard oil, soybean oil, sunflower oil, other oils, phospholipids, surfactants, rofams, and oil-in-water emulsions containing solubilizing agents.

Nanoparticle and nanoemulsion-based systems can be used for delivery of the disclosed peptides/proteins including those based on polyepsilon caprolactone, N-isopropylacrylamide, vinyl pyrrolidone, acrylic acid, Eudragit® RS 100, Eudragit® RL100, poly (lactic/glycolic) acid, and Novasorb™. Cationic nanoemulsions coated with poly-L-lysine, alginate or chitosan stabilize the nanoemulsion and facilitate its interaction with the corneal membrane. Other cationic lipids and excipients appropriate for peptide/protein nanoemulsions include stearlyamine, oleylamine, polyethylenimine, N-(1-(2,3-dioleoyloxy)propyl)-N,N,N trimethylammonium (DOTAP), dioleoyl phosphatidylethanolamine (DOPE), cetrimide, benzalkonium chloride, benzethonium chloride, cetalkonium chloride, benzododecinium chloride, and/or cetylpyridinium. Other nano- or micro-emulsion components that can be used in the topical compositions include osmotic agents (mannitol, glycerol, sorbitol, propylene glycol, and/or dextrose); oils (medium chain triglycerides, triacetin, mineral oil and/or vegetable oils); and/or surfactants (polysorbates, cremephores, poloxamers, tyloxapol, and/or vitamin-E-TPGS).

Polydisperse carrier solutions can be used for the pharmaceutical compositions including Sophisen™, 3A Ofteno™, and Modusik-A Ofteno™.

Liposomal formulations, including cationic liposomes, can be used as carriers for the disclosed peptides/proteins, and include 1-alpha-dimyristoylphosphatidylglycerol, phospholipid, cholesterol, Span™ 40, stearylamine, and/or deoxycholic acid.

Topical compositions such as biodegradable polymers can be used for the sustained release of the disclosed peptides/proteins following mucosal delivery. Examples include gelatin, albumin, polyorthoesters, polyanhydrides, polyvinyl alcohol, polyesters, polymers of D-, L- and DL-lactic acid, and copolymers of lactic and glycolic acid.

Additionally, the disclosed peptides/proteins can be formulated as sustained-release systems for mucosal delivery utilizing semipermeable matrices of solid polymers. Various sustained-release materials have been established and are well known by those of ordinary skill in the art.

Pharmaceutical compositions disclosed herein can also utilize microencapsulation (see, e.g., U.S. Pat. Nos. 4,352,883, 4,353,888, and 5,084,350); continuous release polymer implants (see, e.g., U.S. Pat. No. 4,883,666); and macroencapsulation (see, e.g., U.S. Pat. Nos. 5,284,761, 5,158,881, 4,976,859, and 4,968,733 and published PCT patent applications WO92/19195, WO95/05452).

For buccal administration the topical compositions can take the form of tablets or lozenges formulated in a conventional manner.

For nasal or pulmonary administration or any other administration by inhalation, the disclosed peptides/proteins can be conveniently delivered in the form of an aerosol spray presentation for pressurized packs or a nebulizer, with the use of suitable propellant, e.g., dichlorodifluoromethane, trichlorofluoromethane, dichlorotetra-fluoroethane, carbon dioxide, or other suitable gas or mixture of gases.

Topical composition attributes like pH, osmolality, and viscosity can be adjusted to stabilize the disclosed peptides/proteins; to provide for efficient delivery and to increase subject comfort. Buffers can be added to the pharmaceutical compositions to adjust the pH to a range of between 4.5 and 11.5, and generally within the range of [6.5][disclosed composition have a pH of 6; lower to 5.5?] to 8.5.

“Topical composition in dosage unit form” means physically discrete coherent units suitable for medical administration, each containing a therapeutically effective amount, or a multiple (up to four times) or sub-multiple (down to a fortieth) of a therapeutically effective amount of a disclosed peptide/protein with a dermatological base and/or pharmaceutically acceptable carrier. Whether the topical composition contains a daily dose, or for example, a half, a third or a quarter of a daily dose, will depend on whether the topical composition is to be administered once or, for example, twice, three times or four times a day, respectively.

The amount and concentration of a disclosed peptide/protein in a topical pharmaceutical composition can be selected based on clinically relevant factors, the solubility of the peptide/protein in the carrier, the potency and activity of the peptide, and the manner of administration of the topical pharmaceutical composition. It is only necessary that the peptide/protein constitute a therapeutically effective amount, i.e., such that a suitable effective dosage will be consistent with the dosage form employed in single or multiple unit doses.

The topical pharmaceutical compositions will generally contain from 0.0001 to 99 wt. %, preferably 0.001 to 50 wt. %, more preferably 0.01 to 10 wt. % of the disclosed peptide/protein by weight of the total composition.

In various embodiments, the disclosed peptide/protein can be present at an amount from 0.001 mg/ml to 500 mg/ml. In additional embodiments, the peptide/protein can be provided in an amount of 0.001, 0.01, 0.1, 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 150, 200, 250, 300, 350, 400, 450 or 500 mg/ml.

One exemplary topical pharmaceutical composition with optimal stability and solubility for use in the methods disclosed herein includes the components shown in Table 3:

TABLE 3
Example Pharmaceutical Composition

| Component | Concentration | Purpose |
|---|---|---|
| peptide (e.g., ShK186, | 4.5 mg/mL | Active agent |
| 198 or 192) | | |
| Sodium phosphate | 9 mM | Buffering agent |
| NaCl | 0.72% w/v | Tonicity modifier |
| Polysorbate 20 | 0.045% (w/v) | Surfactant |
| Dimethylsulfoxide | 10% | Penetration enhancer |

pH of 6.0

Other peptides/proteins can be used with the same, or similar, formulations.

In addition to the peptide/protein, the topical pharmaceutical compositions can also contain other drug(s) or biologically-active agent(s). Examples of other drugs or biologically-active agents can include an analgesic agent, a cytokine, and/or a therapeutic agent in all of the major areas of clinical medicine. A cocktail is a mixture of any one of the disclosed peptides/proteins with another drug or biologically-active agent. In this embodiment, a common administration vehicle can contain both the peptides/proteins in combination with the other drugs or biologically-active agent(s).

The topical compositions of the invention comprising a safe and effective amount of a disclosed peptide/protein in an appropriate formulation can be filled and packaged into a plastic squeeze bottle or tube. Suitable container-closure systems for packaging the topical formulations of the invention are commercially available for example, from Wheaton Plastic Products, 1501 North 10th Street, Millville, N.J. 08332.

Preferably, instructions are packaged with the formulations of the present disclosure, for example, a pamphlet or package label. The labeling instructions explain how to administer a topical formulation of the present disclosure, in an amount and for a period of time sufficient to treat or prevent inflammation of the skin or mucosa and signs and/or symptoms associated therewith. Preferably, the label includes the dosage and administration instructions, the topical formulation's composition, the clinical pharmacology, drug resistance, pharmacokinetics, absorption, bioavailability, and contraindications.

The term “topically administrable composition,” a “topical composition,” a “pharmaceutical composition,” a “topical formulation,” or a “formulation” as used herein, means any formulation or composition which is pharmaceutically and/or cosmetically acceptable for topical or mucosal delivery of the specified compounds according to embodiments of the present disclosure. Exemplary forms of a formulation that can be used for topical administration in embodiments of the present disclosure include, but are not limited to, sprays, mists, aerosols, solutions, lotions, gels, creams, ointments, pastes, unguents, emulsions suspensions, thin films, and/or buccal delivery systems.

As used herein, the term “composition” is intended to encompass a product comprising the specified ingredient in the specified amount, as well as any product which results, directly or indirectly, from combinations of the specified ingredient in the specified amount.

As used herein, the term “instructions” when used in the context of a packaged product includes a publication, a recording, a diagram or any other medium of expression which can be used to communicate the usefulness of the packaged product for its designated use. The instructions can, for example, be affixed to or included within a container for the packaged product.

As used herein, the term “treatment” or “treating” refers to an amelioration, prophylaxis, or reversal of erythema or a symptom associated therewith, for example, by lessening or delaying the onset of the erythema, induration, scaling, blistering or other inflammatory symptoms of the skin or mucosa.

As used herein, a “safe and effective amount of a disclosed peptide/protein” means the amount of the peptide that is effective to treat inflammation or a symptom associated therewith, without causing unacceptable drug related adverse events, when administered to a subject.

As used herein, the phrase “unacceptable drug related adverse events,” “unacceptable adverse drug events,” and “unacceptable adverse drug reaction,” shall all mean harm or undesired outcome associated with or caused by a proposed use of a drug, and the harm or undesired outcome reaches such a severity that a regulatory agency deems the drug unacceptable for the proposed use.

Methods of Use

The topical pharmaceutical compositions of the present disclosure are useful in methods of treating psoriasis, atopic dermatitis and other inflammatory skin conditions. Such methods include administering to a subject in need thereof a therapeutically effective amount of a topical pharmaceutical composition that includes a disclosed peptide/protein.

Topical or mucosal inflammatory conditions (or diseases characterized by skin or mucosal inflammatory manifestations) that can be treated according to the methods disclosed herein include cutaneous lupus, dermatomyositis, scleroderma, psoriasis, atopic dermatitis, vasculitis, Bechet's syndrome, Henoch-Schonlein prupura, hypersensitivity reactions, Kawasaki disease, microscopic polyangitis, polyarteritis nodosa, vitiligo, alopecia areata, autoimmune progesterone dermatitis, Henoch-Schonlein purpura, Blau syndrome, bullous penphigoid, Churg-Strauss syndrome, cicatricial pemphigoid, contact dermatitis, Chron's disease, inflammatory bowel disease, dermatitis herpetiformis, diffuse cutaneous systemic sclerosis, discoid lupus erythematosus, ecaema, eosinophilic fasciitis, epidermolysis bullosa acquisita, erythema nodosum, lichen planus, lupus erythematosus, Majeed syndrome, morphea, Mucha-Habermann disease, Parry-Romberg syndrome, POEMS syndrome, psoriasis, pyoderma gangrenosum, relapsing polychondritis, reactive arthritis, rheumatoid arthritis, scleroderma, Sjogren's syndrome, systemic lupus erythematosis, vasculitis and Wegener's granulomatosis, microscopic polyangiitis, polyarteritis nodosa, Takayasu's arteritis, rheumatoid arthritis, relapsing polychondritis and ankylosing spondylitis, Graves disease, Hashimoto's thyroiditis, and Sezary syndrome.

Methods disclosed herein include treating subjects (humans, veterinary animals (dogs, cats, reptiles, birds, etc.), livestock (horses, cattle, goats, pigs, chickens, and the like), and research animals (monkeys, rats, mice, fish, and the like) with topical pharmaceutical compositions disclosed herein. Treating subjects includes delivering therapeutically effective amounts of the topical pharmaceutical compositions. Therapeutically effective amounts include those that provide effective amounts, prophylactic treatments, and/or therapeutic treatments.

An “effective amount” is the amount of a topical pharmaceutical composition necessary to result in a desired physiological change in the subject. Effective amounts are often administered for research purposes. Effective amounts disclosed herein result in a desired physiological change in a research assay intended to study the effectiveness of a topical pharmaceutical composition in the treatment of skin or mucosal inflammation. Effective amounts can reduce the population of T_{EM} (i.e., reduce proliferation); reduce activation of T_{EM} as measured by cytokine production (e.g., IFN-γ; IL-2; IL-4; IL-10; IL-17 and IL-21) and/or perforin production; and/or reduce expression of Kv1.3 channels. Reductions can be seen based on comparisons to a reference level from a previous measure from the same subject or as compared to a reference level obtained from a dataset from a population.

A “prophylactic treatment” includes a treatment administered to a subject who does not display signs or symptoms of skin or mucosal inflammation or displays only early signs or symptoms such that treatment is administered for the purpose of diminishing, preventing, or decreasing the risk of developing the condition(s) further. Thus, a prophylactic treatment functions as a preventative treatment against the condition(s).

A “therapeutic treatment” includes a topical treatment administered to a subject who displays symptoms or signs of skin or mucosal inflammation and is topically administered to the subject for the purpose of diminishing or eliminating those signs or symptoms of the condition(s). The therapeutic treatment can reduce, control, or eliminate the presence or activity of the condition(s) and/or reduce, control or eliminate side effects of the condition(s).

For administration, effective amounts and therapeutically effective amounts (also referred to herein as doses) can be initially estimated based on results from in vitro assays and/or animal model studies. For example, a dose can be formulated in animal models to achieve a local concentration range that includes an IC₅₀ as determined in cell culture against activation, proliferation, cytokine production, and/or perforin production by T_{EM}. Such information can be used to more accurately determine useful doses in subjects of interest.

The actual amount administered to a particular subject as a therapeutically effective amount can be determined by a physician, veterinarian, or researcher taking into account parameters such as physical and physiological factors including target, body weight, severity of condition, type of condition, previous or concurrent therapeutic interventions, and idiopathy of the subject.

Dosage can be adjusted appropriately to achieve a desired peptide/protein level locally. Typically the peptides/proteins of the present disclosure exhibit their effect at a dosage range from 0.001 mg/kg to 250 mg/kg, preferably from 0.01 mg/kg to 100 mg/kg of the peptide/protein, more preferably from 0.05 mg/kg to 75 mg/kg. A suitable dose can be administered in multiple sub-doses per day. Typically, a dose or sub-dose can contain from 0.1 mg to 500 mg of the peptide/protein per unit dosage form. A more preferred dosage will contain from 0.5 mg to 100 mg of the peptide/protein per unit dosage form.

Additional useful doses often range of 0.001 to 10,000 micrograms (μg) of the peptide/protein per kilogram (kg) of body mass, in the range of 1 to 5,000 μg/kg of body mass, in the range of 1 to 1,000 gig/kg of body mass or in the range of 1 to 100 μg/kg of body mass. Often, doses can range from 0.1 to 5 μg/kg or from 0.5 to 1 μg/kg. In other examples, a dose can include 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1,000 μg/kg, 1,500 μg/kg, 2,000 μg/kg, 2,500 μg/kg, 3,000 μg/kg, 3,500 μg/kg, 4,000 μg/kg, 5,000 μg/kg, 6,000 μg/kg, 7,000 μg/kg, 8,000 μg/kg, 9,000 μg/kg, 10,000 μg/kg, 0.1 to 5 mg/kg or from 0.5 to 1 mg/kg. In other examples, a dose can include 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, or 50 mg/kg.

When cocktails are used, a common administration vehicle can contain both the peptides/proteins in combination with the other drugs or agents. The individual components of the cocktail can each be administered in therapeutically effective amounts or their administration in combination can create a therapeutically effective amount.

In particular embodiments, dosages can be initiated at lower levels and increased until desired effects are achieved. In the event that the response in a subject is insufficient at such doses, even higher doses (or effective higher doses by a different, more localized delivery route) may be employed to the extent that subject tolerance permits. Continuous applications over, for example, 24 hours or multiple doses per day are contemplated to achieve appropriate tissue-specific levels of peptide/protein.

Therapeutically effective amounts can be achieved by administering single or multiple doses during the course of a treatment regimen (e.g., daily, twice-daily, thrice-daily, four times daily, five times daily, six times daily, seven times daily, eight times daily, nine times daily, ten times daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 month, or yearly.

The topical pharmaceutical composition can be administered in conjunction with one or more systemic or topical products that alleviate symptoms of the condition such as tacrolimus, pimecrolimus or corticosteroids.

The present disclosure also provides methods of screening subjects with skin or mucosal inflammation to assess the potential therapeutic benefit of the methods and topical compositions disclosed herein in the screened subjects. In one embodiment, levels of expression of Kv1.3 channels in the subject's T-cell populations are assessed using an antibody that detects surface expression of the channel. An anti-potassium channel Kv1.3 (extracellular) antibody to practice these methods is available from Alomone (Israel); antibodies are also available from LifeSpan Biosciences, Inc. (Seattle, Wash., USA). Kv1.3 channel levels can be indicative of an inflammatory condition that can be effectively treated with a peptide/protein disclosed herein.

To identify subjects that will benefit from the methods and topical compositions disclosed herein, the subject's Kv1.3 channel levels are compared to a reference level obtained from a dataset. A reference level from a dataset can be derived from previous measures in the same subject or can be derived from a population.

A “population” is any grouping of subjects of like specified characteristics. The grouping could be according to, for example, clinical parameters, clinical assessments, therapeutic regimens, disease status (healthy or having an inflammatory condition disclosed herein), severity of inflammation, and the like.

A “dataset” as used herein is a set of numerical values resulting from evaluation of a sample (or a population of samples) under a desired condition. The values of the dataset can be obtained, for example, by experimentally obtaining measures from a sample and constructing a dataset from these measurements. As is understood by one of ordinary skill in the art, the reference level can be based on e.g., any mathematical or statistical formula useful and known in the art for arriving at a meaningful aggregate reference level from a collection of individual data points; e.g., mean, median, median of the mean, etc. Alternatively, a reference level or dataset to create a reference level can be obtained from a service provider such as a laboratory, or from a database or a server on which the dataset has been stored.

In particular embodiments, a subject can be selected as one who will benefit from the treatments disclosed herein based on Kv1.3 channel levels that are not statistically significantly different from a reference level from a population that previously benefited from the disclosed treatments. In additional embodiments, a subject can be selected as one who will benefit from the treatments disclosed herein based on Kv1.3 channel levels that are not statistically significantly different from a reference level from population having an inflammatory condition disclosed herein. In further particular embodiments, a subject can be selected as one who will benefit from the treatments disclosed herein based on Kv1.3 channel levels that are statistically significantly higher over a reference level from a healthy population.

Kv1.3 channel levels are not significantly different if the difference is within a level that would be expected to occur based on chance alone. In contrast, a statistically significant difference or increase is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular data point, where the data point is the result of random chance alone. A result is often considered significant (not random chance) at a p-value less than or equal to 0.05.

The described screening methods can be used to direct a subject's treatment. For example, if the subject's Kv1.3 channel levels identify the subject as one who would benefit from the methods and topical compositions disclosed herein, the subject can be prescribed or given a therapeutically effective amount of a topical pharmaceutical composition disclosed herein. The results of the screening methods can also be used to, for example, provide clinical decision support, such as determining whether to defer intervention or treatment, to recommend preventive check-ups for at-risk patients, to recommend increased visit frequency, to recommend increased testing, and/or to recommend intervention. The results of the methods can also be useful for therapeutic selection, determining response to treatment, adjustment, dosing levels, and application of treatment, monitoring ongoing therapeutic efficiency, and indication for change in therapeutic regimens.

Methods of Manufacture

The disclosed peptides/proteins can be prepared using recombinant DNA technology. The peptides/proteins can also be prepared using, for example, the Merrifield solid-phase synthesis, although other equivalent chemical syntheses known in the art can also be used. Solid-phase synthesis is commenced from the C-terminus of the peptide/protein by coupling a protected α-amino acid to a suitable resin. Such a starting material can be prepared by attaching an α-amino-protected amino acid by an ester linkage to a chloromethylated resin or a hydroxymethyl resin, or by an amide bond to a benzhydrylamine (BHA) resin or para-methylbenzhydrylamine (MBHA) resin. Preparation of the hydroxymethyl resin is described by Bodansky et al., Chem. Ind. (London) 38, 1597 (1966). Chloromethylated resins are commercially available from, for example, Bio Rad Laboratories (Richmond, Calif.) and from Lab. Systems, Inc. The preparation of such a resin is described by Stewart & Young, Solid phase peptide synthesis. W.H. Freeman, Kent, England (1969). BHA and MBHA resin supports are commercially available, and are generally used when the desired peptide/protein being synthesized has an unsubstituted amide at the C-terminus. Thus, solid resin supports may be any of those known in the art, such as one having the formulae —O—CH₂-resin support, —NH BHA resin support, or —NH-MBHA resin support. When the unsubstituted amide is desired, use of a BHA or MBHA resin can be advantageous because cleavage directly gives the amide. Where the N-methyl amide is desired, it can be generated from an N-methyl BHA resin. Should other substituted amides be desired, the teaching of U.S. Pat. No. 4,569,967 can be used, or should still other groups than the free acid be desired at the C-terminus, it can be preferable to synthesize the peptide/protein using classical methods as set forth in Houben & Weyl, Methoden der organischen Chemie, Georg Theime, Stuttgart (1974).

The C-terminal amino acid, protected by Boc or Fmoc and by a side-chain protecting group, if appropriate, can be first coupled to a chloromethylated resin according to the procedure set forth in Horiki et al., Chem. Lett., 165-168, (1978) using potassium fluoride (KF) in dimethylformamide (DMF) at about 60° C. for 24 hours with stirring, when a peptide/protein having free acid at the C-terminus is to be synthesized. Following the coupling of the BOC-protected amino acid to the resin support, the α-amino protecting group can be removed, as by using trifluoroacetic acid (TFA) in methylene chloride CH2Cl2 or TFA alone. The deprotection can be carried out at a temperature between 0° C. and room temperature. Other standard cleaving reagents, such as HCl in dioxane, and conditions for removal of specific α-amino protecting groups can be used as described in Schroeder & Lubke, The Peptides, Academic Press: New York (1965).

After removal of the α-amino-protecting group, the remaining α-amino- and side chain-protected amino acids can be coupled step-wise in the desired order to obtain an intermediate compound or as an alternative to adding each amino acid separately in the synthesis, some of them can be coupled to one another prior to addition to the solid phase reactor. Selection of an appropriate coupling reagent is within the skill of the art. Exemplary coupling reagents include N,N′-dicyclohexylcarbodiimide (DCC), diisopropylcarbodiimide (DIC), O-(benzotriazol-1-yl)-N,N,N′,N′-tetramethyluronium hexafluorophosphate (HBTU), O-(7-azabenzotriazol-1-yl)-N,N,N′,N′-tetramethyluronium hexafluorophosphate (HATU), O-(benzotriazol-1-yl)-N,N,N′,N′-tetramethyluronium tetrafluoroborate (TBTU) in the presence of 1-hydroxybenzotriazole (HoBt) or 1-Hydroxy-7-azabenzotriazole (HoAt)).

The activating reagents used in the solid phase synthesis of peptides including the disclosed peptides/proteins are well known in the art. Examples of suitable activating reagents include carbodiimides, such as N, N′-diisopropylcarbodiimide (DIC) and N-ethyl-N′-(3-dimethylaminopropyl)carbodiimide (EDAC). Other activating reagents and their use in peptide coupling are described by Schroeder & Lubke, The Peptides, Academic Press: New York (1965) and Kapoor, J. Pharm. Sci., 59(1), 1-27 (1970).

Each protected amino acid or amino acid sequence can be introduced into the solid-phase reactor in a twofold or more excess, and the coupling may be carried out in a medium of DMF:CH₂Cl₂ (1:1) or in DMF or CH₂Cl₂ alone. In cases where intermediate coupling occurs, the coupling procedure can be repeated before removal of the α-amino protecting group prior to the coupling of the next amino acid. The success of the coupling reaction at each stage of the synthesis, if performed manually, can be monitored by, for example, the ninhydrin reaction, as described by Kaiser et al., Anal. Biochem. Vol 34(2), 595-8 (1970).

Coupling reactions can be performed automatically, as, for example, on a Beckman 990 automatic synthesizer, using a program such as that reported in Rivier et al., Biopolymers, 17(8), 1927-1938 (1978) or on a Applied Biosystems 433A peptide synthesizer.

After the desired amino acid sequence has been completed, the intermediate peptide can be removed from the resin support by treatment with a reagent, such as liquid hydrogen fluoride or TFA (if using Fmoc chemistry), which not only cleaves the peptide from the resin but also cleaves all remaining side chain protecting groups and also the α-amino protecting group at the N-terminus if it was not previously removed to obtain the peptide in the form of the free acid. If Met is present in the sequence, the Boc protecting group can be first removed using TFA/ethanedithiol prior to cleaving the peptide from the resin with HF to eliminate potential S-alkylation. When using hydrogen fluoride or TFA for cleaving, one or more scavengers such as anisole, cresol, dimethyl sulfide and methylethyl sulfide can be included in the reaction vessel.

Cyclization of a linear peptide/protein can be affected, as opposed to cyclizing the peptide/protein while a part of the peptido-resin, to create bonds between Cys residues. To effect such a disulfide cyclizing linkage, a fully protected peptide/protein can be cleaved from a hydroxymethylated resin or a chloromethylated resin support by ammonolysis, as is well known in the art, to yield the fully protected amide intermediate, which is thereafter suitably cyclized and deprotected. Alternatively, deprotection, as well as cleavage of the disclosed peptide/protein from the above resins or a benzhydrylamine (BHA) resin or a methylbenzhydrylamine (MBHA), can take place at 0° C. with hydrofluoric acid (HF) or TFA, followed by oxidation as described above.

The disclosed peptides/proteins can also be synthesized by sequentially adding amino acids coupled to an MBHA Rink resin (typically 100 mg of resin) beginning at the C-terminus using an Advanced Chemtech 357 Automatic Peptide Synthesizer. Couplings are carried out using 1,3-diisopropylcarbodimide in N-methylpyrrolidinone (NMP) or by 2-(1H-benzotriazole-1-yl)-1,1,3,3-tetramethyluronium hexafluorophosphate (HBTU) and diethylisopropylethylamine (DIEA). The Fmoc protecting group can be removed by treatment with a 20% solution of piperidine in dimethylformamide (DMF). Resins are subsequently washed with DMF (twice), followed by methanol and NMP.

### Exemplary Embodiments

1. A method of treating inflammation of the skin or mucosa in a subject in need thereof including administering to the subject a therapeutically effective amount of a topical pharmaceutical composition including a peptide having at least 80% sequence identity to any of SEQ ID NOs: 1-260.

2. A method of embodiment 1, wherein the peptide has at least 85% sequence identity to any of SEQ ID NOs:1-260.

3. A method of embodiments 1 or 2, wherein the peptide has at least 90% sequence identity to any of SEQ ID NOs: 1-260.

4. A method of any of embodiments 1-3, wherein the peptide has at least 95% sequence identity to any of SEQ ID NOs: 1-260.

5. A method of any of embodiments 1-4, wherein the peptide has at least 96% sequence identity to any of SEQ ID NOs: 1-260.

6. A method of any of embodiments 1-5, wherein the peptide has at least 97% sequence identity to any of SEQ ID NOs: 1-260.

7. A method of any of embodiments 1-6, wherein the peptide has at least 98% sequence identity to any of SEQ ID NOs: 1-260.

8. A method of any of embodiments 1-7, wherein the peptide has at least 99% sequence identity to any of SEQ ID NOs: 1-260.

9. A method of embodiment 1, wherein the peptide is a peptide having at least 80% sequence identity to any of SEQ ID NOs:225-256.

10. A method of embodiment 9, wherein the peptide has at least 85% sequence identity to any of SEQ ID NOs:225-256.

11. A method of embodiments 9 or 10, wherein the peptide has at least 90% sequence identity to any of SEQ ID NOs:225-256.

12. A method of any of embodiments 9-11, wherein the peptide has at least 95% sequence identity to any of SEQ ID NOs:225-256.

13. A method of any of embodiments 9-12, wherein the peptide has at least 96% sequence identity to any of SEQ ID NOs:225-256.

14. A method of any of embodiments 9-13, wherein the peptide has at least 97% sequence identity to any of SEQ ID NOs:225-256.

15. A method of any of embodiments 9-14, wherein the peptide has at least 98% sequence identity to any of SEQ ID NOs:225-256.

16. A method of any of embodiments 9-15, wherein the peptide has at least 99% sequence identity to any of SEQ ID NOs:225-256.

17. A method of embodiment 1, wherein the peptide is an ShK-based peptide having at least 80% sequence identity to any of SEQ ID NOs:1-224 and SEQ ID NOs:257-260.

18. A method of embodiment 17, wherein the ShK-based peptide has at least 85% sequence identity to any one of SEQ ID NOs:1-224 and SEQ ID NOs:257-260.

19. A method of embodiments 17 or 18, wherein the ShK-based peptide has at least 90% sequence identity to any one of SEQ ID NOs: 1-224 and SEQ ID NOs:257-260.

20. A method of any one of embodiments 17-19, wherein the ShK-based peptide has at least 95% sequence identity to any one of SEQ ID NOs:1-224 and SEQ ID NOs:257-260.

21. A method of any one of embodiments 17-20, wherein the ShK-based peptide has at least 96% sequence identity to any one of SEQ ID NOs:1-224 and SEQ ID NOs:257-260.

22. A method of any one of embodiments 17-21, wherein the ShK-based peptide has at least 97% sequence identity to any one of SEQ ID NOs:1-224 and SEQ ID NOs:257-260.

23. A method of any one of embodiments 17-22, wherein the ShK-based peptide has at least 98% sequence identity to any one of SEQ ID NOs:1-224 and SEQ ID NOs:257-260.

24. A method of any one of embodiments 17-23, wherein the ShK-based peptide has at least 99% sequence identity to any one of SEQ ID NOs:1-224 and SEQ ID NOs:257-260.

25. A method of any one of embodiments 1 and 17-20, wherein the ShK-based peptide has at least 95% sequence identity to SEQ ID NO: 1.

26. A method of any one of embodiments 1 and 17-20, wherein the ShK-based peptide has at least 95% sequence identity to SEQ ID NO:2.

27. A method of any one of embodiments 1 and 17-20, wherein the ShK-based peptide has at least 95% sequence identity to SEQ ID NO:208, SEQ ID NO:209, SEQ ID NO:210, SEQ ID NO:217, and/or SEQ ID NO:218.

28. A method of any one of embodiments 1, 17-20, and 27, wherein the ShK-based peptide has at least 96% sequence identity to SEQ ID NO:208, SEQ ID NO:209, SEQ ID NO:210, SEQ ID NO:217, and/or SEQ ID NO:218.

29. A method of any one of embodiments 1, 17-20, 27, and 28, wherein the ShK-based peptide has at least 97% sequence identity to SEQ ID NO:208, SEQ ID NO:209, SEQ ID NO:210, SEQ ID NO:217, and/or SEQ ID NO:218.

30. A method of any one of embodiments 1, 17-20, and 27-29, wherein the ShK-based peptide has at least 98% sequence identity to SEQ ID NO:208, SEQ ID NO:209, SEQ ID NO:210, SEQ ID NO:217, and/or SEQ ID NO:218.

31. A method of any one of embodiments 1, 17-20, and 27-30, wherein the ShK-based peptide has at least 99% sequence identity to SEQ ID NO:208, SEQ ID NO:209, SEQ ID NO:210, SEQ ID NO:217, and/or SEQ ID NO:218.

32. A method of any one of embodiments 1-31, wherein the peptide is natural or synthetic.

33. A method of any one of embodiments 1-32, wherein the peptide is attached to an organic or inorganic chemical entity that has an anionic charge.

34. A method of any one of embodiments 1-33, wherein the C-terminus of the peptide is an acid or an amide.

35. A method of any one of embodiments 1-34, wherein the topical pharmaceutical composition is administered to the skin or mucosa.

36. A method of any one of embodiments 1-36, wherein the topical pharmaceutical composition is administered six times daily, five times daily, four times daily, three times daily, twice daily, daily, weekly, monthly, every two months, every three months, or every six months.

37. A method of any one of embodiments 1-36, wherein the skin or mucosal inflammatory condition is or is caused by cutaneous lupus, dermatomyositis, scleroderma, psoriasis, atopic dermatitis, vasculitis, Bechet's syndrome, Henoch-Schonlein prupura, hypersensitivity reactions, Kawasaki disease, microscopic polyangitis, polyarteritis nodosa, vitiligo, alopecia areata, autoimmune progesterone dermatitis, Henoch-Schonlein purpura, Blau syndrome, bullous penphigoid, Churg-Strauss syndrome, cicatricial pemphigoid, contact dermatitis, Crohn's disease, inflammatory bowel disease, dermatitis herpetiformis, diffuse cutaneous systemic sclerosis, discoid lupus erythematosus, ecaema, eosinophilic fasciitis, epidermolysis bullosa acquisita, erythema nodosum, lichen planus, lupus erythematosus, Majeed syndrome, morphea, Mucha-Habermann disease, Parry-Romberg syndrome, POEMS syndrome, psoriasis, pyoderma gangrenosum, relapsing polychondritis, reactive arthritis, rheumatoid arthritis, scleroderma, Sjogren's syndrome, systemic lupus erythematosis, vasculitis and Wegener's granulomatosis, microscopic polyangiitis, polyarteritis nodosa, Takayasu's arteritis, rheumatoid arthritis, relapsing polychondritis or ankylosing spondylitis.

38. A method of any one of embodiments 1-37, wherein the subject is a human adult, child or adolescent.

39. A method of any one of embodiments 1-38, wherein the topical pharmaceutical composition comprises a dermatological base.

40. A method of embodiment 39, wherein the dermatological base is water, ethanol, 2-propanol, glycerol, propylene glycol, sorbitol, macrogol, dimethyl sulfoxide, acetone, petrolatum, hard paraffin, soft and liquid paraffin, triglycerides, wax, liquid wax ester, partial glycerides, silicon oils, anionic surfactants, zwitterionic surfactants, nonionic surfactants, bentonite, carbomer, carmellose sodium, hydroxyethyl cellulose, hydroxypropyl cellulose, hypromellose, alkyl-4-hydroxybenzoates, sorbic acid, benzyl alcohol, phenylethyl alcohol, butylhydroxyanisol, butylated hydroxytoluene, tocopherol, tocopherol acetate and ascorbyl palmitate propylene glycol, yellow wax, fluid wax esters, isopropyl myristate, ethylhexyl palmitate, dimethicone, phenyl methyl polysiloxane and/or cyclomethicone.

41. A method of embodiments 1-40, wherein the topical pharmaceutical composition comprises an emulsifying agent.

42. A method of embodiment 41, wherein the emulsifying agent is sodium stearate, aluminum stearate, sodium dodecyl sulfate, sodium cetyl stearyl sulfate, sodium lauryl ether sulfate, sodium dioctyl sulfosuccinate, cetyl trimethyl ammonium bromide, benzalkonium bromide, cetylpyrdinium chloride, phosphatidylcholine, betaine monohydrate, and Dehyton K®, PEG-30 stearate, glycerol monostearate, glycerol monooleate, glycerol monoisostearate, partial glyceride medium chain, Tween®, polyoxyethylene sorbitan fatty acid esters, polyoxyethylene (20) sorbitan monostearate, sorbitan laurate, sorbitan monooleate, sorbitan monopalmitate, sorbitan tristearate, sorbitan sesquiloleate, saccharose monostearate, saccharose cocoate, cetomagrogol 1000, macrogol cetostearyl ether, macrogol oleyl ether, lauromacrogol 400, cholesterol, wool fat, acetylated wool fat, hydrated wool fat, wool fat alcohols, macrogol 1000 glycerol monooleate, macrogol 1000 glycerol monostearate, macrogol 300 glycerol tris hydroxystearate, macrogol 5 glycerol stearate, macrogol glycerol hydroxystearate, and/or triglycerol diisostearate.

43. A method of embodiments 1-42, wherein the topical pharmaceutical composition comprises a gelling agent.

44. A method of embodiment 43, wherein the gelling agent is synthetic polyacrylic acid (carbomer), a semi-synthetic cellulose derivative, sodium carboxymethylcellulose, hydroxyethyl cellulose, xanthan, inorganic bentonite and/or hypromellose.

45. A method of embodiments 1-44, wherein the topical pharmaceutical composition comprises a preservative.

46. A method of embodiment 45, wherein the preservative is methylalkyl-4-hydroxybenzoate, ethylalkyl-4-hydroxybenzoate, propylalkyl-4-hydroxybenzoate, butylalkyl-4-hydroxybenzoate, paraben, sorbic acid, benzoic acid, benzyl alcohol, phenoxyethanol, and/or phenyl ethyl alcohol.

47. A method of embodiments 1-46, wherein the topical pharmaceutical composition comprises an antioxidant.

48. A method of embodiment 47 wherein the antioxidant is alpha-tocopherol, ethyl gallate, propyl gallate, octyl gallate, lauryl gallate, decyl ester gallate, butylhydroxyanisol, butylated hydroxytoluene, potassium ascorbate, sodium ascorbate, calcium ascorbate, ascorbyl myristate, ascorbyl palmitate, ascorbyl stearate, ethylenediaminetetraacetic acid, sodium bisulfite, sodium metabisulfite, and/or thiourea.

49. A method of any one of embodiments 1-48, wherein the topical pharmaceutical composition comprises a penetration enhancer.

50. The method of embodiment 49, wherein the penetration enhancer is a sulphoxide, a azone, a pyrrolidone, an alcohol or an alkanol, a glycol, a surfactant and/or a terpene.

51. The method of embodiment 50, wherein the sulfoxide is dimethyl sulfoxide, the azone is lauropram, the pyrrolidine is 2-pyrrolidone, the alcohol is ethanol, 2-propanol, sorbitol, benzyl alcohol, or phenylethyl alcohol, the glycol is propylene glycol, macrogol, or ascorbyl palmitate propylene glycol, the surfactant is a anionic surfactant, a zwitterionic surfactant or a nonionic surfactant, and the terpene is 1,8-cineole, menthone, (+)-limonene or nerolidol.

52. A method of any one of embodiments 1-48, wherein the topical pharmaceutical composition comprises a penetration enhancer, a buffering agent, a tonicity modifier, and a surfactant.

53. A method of any one of embodiments 1-49, wherein the topical pharmaceutical composition comprises dimethyl sulfoxide at at least 5%, at least 10%, at least 15%, at least 20%, at least 25%, at least 30%, at least 35%, at least 40%, at least 45%, at least 50%, at least 55%, or at least 60%, 10 mM sodium phosphate; 0.8% w/v NaCl; and Polysorbate 20 at 0.01, 0.05, 0.1, 0.2, 0.4, 0.6, 0.8, 1, 2, 3, or 4 w/v %, wherein the composition has a pH of 5.0, 5.5, 6.0, 6.5, 7, 7.5, or 8.

54. A method of any one of embodiments 1-51, wherein the topical pharmaceutical composition includes Polysorbate 20 at 0.05 w/v %, and wherein the composition has a pH of 6.0.

55. A method of any one of embodiments 1-51, wherein the topical pharmaceutical composition includes 10 mM sodium phosphate; 0.8% w/v NaCl; and Polysorbate 80 at 0.01, 0.05, 0.1, 0.2, 0.4, 0.6, 0.8, 1, 2, 3, or 4 w/v %, wherein the composition has a pH of 5.0, 5.5, 6.0, 6.5, 7, 7.5, or 8.

56. A method of any one of embodiments 1-51 or 55, wherein the pharmaceutical composition includes Polysorbate 80 at 0.05 w/v %, and wherein the composition has a pH of 6.0.

57. A method of any one of embodiments 1-56, wherein the therapeutically effective amount the peptide having at least 80% sequence identity to any of SEQ ID NOs: 1-260 decreases inflammation of the skin or mucosa including erythema, induration, scaling, itching, blistering, ulcerating, dryness, bleeding, peeling and/or necrotization.

58. A method of evaluating a subject to predict the outcome of treatment with a method of any one of embodiments 1-56 including: analyzing Kv1.3 channel expression levels of T-cells from a biological sample of the subject; wherein increased levels of Kv1.3 channel expression relative to a healthy control or reference population is indicative of a patient receptive to treatment with a peptide having at least 80% sequence identity to any one of SEQ ID NOs: 1-224 and SEQ ID NOs:257-260.

59. A method of screening for subjects who may benefit from treatment with a method of any one of embodiments 1-56 including: measuring Kv1.3 channel expression levels of T-cells and/or macrophages from a biological sample of the subject; comparing Kv1.3 channel expression levels of the subject to that of a healthy control or reference population; and determining that the subject will benefit from treatment with a method of any one of embodiments 1-54 if the level of Kv1.3 channel expression in the subject is increased compared to the healthy control or reference population.

60. A method of selecting subjects for a clinical trial including: measuring Kv1.3 channel expression levels of T-cells and/or macrophages from a biological sample of the subject; comparing Kv1.3 channel expression levels of the subject to that of a healthy control or reference population; and selecting the subject for the clinical trial if the subject has increased Kv1.3 channel expression levels compared to the healthy control or reference population, or excluding the subject from the clinical trial if the subject has decreased or unchanged Kv1.3 channel expression levels compared to the healthy control or reference population.

61. A method for screening potential treatments for dermatological conditions in a subject including: measuring Kv1.3 channel expression levels of T-cells and/or macrophages from a biological sample of the subject; comparing Kv1.3 channel expression levels of the subject to that of a healthy control or reference population; and identifying any one of the methods of embodiments 1-54 as a treatment for the subject if the subject has increased Kv1.3 channel expression levels compared to the healthy control or reference population.

62. A method of any one of embodiments 59-61, wherein the Kv1.3 channel expression level is measured using an assay.

63. A method of embodiment 58, wherein the Kv1.3 channel expression level is analyzed using an assay.

64. A method of any one of embodiments 58-63, further including challenging the T-cells with a proinflammatory immune stimulator or T-cell activating agent in the presence of a toxin-based therapeutic peptide.

65. A method of embodiment 64, wherein the peptide is an ShK-based peptide.

66. A method of embodiment 65, wherein the peptide has at least 95% sequence identity to SEQ ID NO: 1, SEQ ID NO:2, SEQ ID NO:208, SEQ ID NO:209, SEQ ID NO:210, SEQ ID NO:217, and/or SEQ ID NO:218.

67. A method of any one of embodiments 58-66, further including measuring proinflammatory cytokine production.

68. A method of embodiment 67, wherein the measured cytokine is Interferon (IFN)-γ, Interleukin (IL)-1a, IL-1b, IL-2, IL-4, IL-6, IL-7, IL-8, IL-10, IL-15, IL-17A, IL-17F, IL-17A/F, IL-21, IL-22, IL-23, Granulocyte macrophage colony-stimulating factor (GM-CSF), Tumor necrosis factor (TNF)-α, metalloprotease (MMP)3, and/or MMP9.

69. A method of any one of embodiments 58-68, further including challenging the T-cells with antigens in the presence or absence of a toxin-based therapeutic peptide.

70. A method of embodiment 69, wherein the peptide is an ShK-based peptide.

71. A method of embodiment 70, wherein the peptide has at least 95% sequence identity to SEQ ID NO: 1, SEQ ID NO:2, SEQ ID NO:208, SEQ ID NO:209, SEQ ID NO:210, SEQ ID NO:217, and/or SEQ ID NO:218.

72. A method of embodiment 71, wherein the peptide has the formula SEQ ID NO:217.

73. A method of embodiment 68, wherein the peptide has the formula SEQ ID NO:210.

74. An exemplary embodiment of any one of embodiments 1-73, wherein the dermatological condition is an inflammatory dermatological condition.

75. A composition for use in the topical treatment of inflammation of the skin or mucosa in a subject in need thereof comprising a therapeutically effective amount of at least one peptide having an amino acid sequence at least 95% identical to any of SEQ ID NOs:1-224 and SEQ ID NOs:257-260 and a dermatological base and/or a pharmaceutically acceptable carrier.

76. A composition for use in the topical treatment of inflammation of the skin or mucosa in a subject in need thereof comprising a therapeutically effective amount of a pharmaceutical composition comprising an peptide having an amino acid sequence at least 95% identical to any one of SEQ ID NOs:208-224 and SEQ ID NOs:257-260 and a dermatological base and/or a pharmaceutically acceptable carrier.

77. The composition according to any one of embodiments 75 and 76, wherein the peptide has at least 95% identity to at least one of SEQ ID NO:1, SEQ ID NO:2, SEQ ID NO:208, SEQ ID NO:209, SEQ ID NO:210, SEQ ID NO:217, and SEQ ID NO:218.

78. A composition for use in the topical treatment of inflammation of the skin or mucosa inflammatory condition in a subject in need thereof comprising a therapeutically effective amount of a pharmaceutical composition comprising a peptide having an amino acid sequence at least 95% identical to any one of SEQ ID NOs: 1 to 224 or 257-260, wherein the inflammation of the skin or mucosa is or is caused by cutaneous lupus erythematosis, dermatomyositis, scleroderma, psoriasis, atopic dermatitis, vasculitis, Bechet's syndrome, Henoch-Schonlein prupura, hypersensitivity reactions, Kawasaki disease, microscopic polyangitis, polyarteritis nodosa, vitiligo, alopecia areata, autoimmune progesterone dermatitis, Henoch-Schonlein purpura, Blau syndrome, bullous penphigoid, Churg-Strauss syndrome, cicatricial pemphigoid, contact dermatitis, Crohn's disease, inflammatory bowel disease, dermatitis herpetiformis, diffuse cutaneous systemic sclerosis, discoid lupus erythematosus, ecaema, eosinophilic fasciitis, epidermolysis bullosa acquisita, erythema nodosum, lichen planus, lupus erythematosus, Majeed syndrome, morphea, Mucha-Habermann disease, Parry-Romberg syndrome, POEMS syndrome, psoriasis, pyoderma gangrenosum, relapsing polychondritis, reactive arthritis, rheumatoid arthritis, scleroderma, systemic lupus erythematosis, vasculitis and Wegener's granulomatosis, Takayasu's arteritis, or ankylosing spondylitis.

79. The composition according to any one of embodiments 75 to 78, wherein the dermatological base comprises a penetration enhancer, a hydrophilic emulsifying agent, a hydrophilic gelling agent, a hydrophobic emulsifying agent, a hydrophobic gelling agent, a preservative and/or an antioxidant.

80. The composition according to embodiment 79, wherein the dermatological base further comprises water, ethanol, 2-propanol, glycerol, propylene glycol, sorbitol, macrogol, dimethyl sulfoxide, acetone, petrolatum, hard paraffin, soft and liquid paraffin, triglycerides, wax, liquid wax ester, a partial glyceride, a silicon oil, an anionic surfactant, a zwitterionic surfactant, a nonionic surfactant, bentonite, carbomer, carmellose sodium, hydroxyethyl cellulose, hydroxypropyl cellulose, hypromellose, alkyl-4-hydroxybenzoates, sorbic acid, benzyl alcohol, phenylethyl alcohol, butylhydroxyanisol, butylated hydroxytoluene, tocopherol, tocopherol acetate, ascorbyl palmitate, and/or combinations of any one or more of the above.

81. The composition according to embodiment 80, wherein the hydrophobic base is a yellow wax, a fluid wax esters, or a silicone oil that contains polyorganoziloxane as the functional group.

82. The composition according to embodiment 79, wherein the emulsifier is nonionic, anionic, cationic and/or zwitterionic.

83. The composition according to embodiment 82, wherein the anionic emulsifier is sodium stearate, aluminum stearate, sodium dodecyl sulfate, sodium cetyl stearyl sulfate, sodium lauryl ether sulfate, and/or sodium dioctyl sulfosuccinate.

84. The composition according to embodiment 82, wherein the cationic emulsifier is cetyl trimethyl ammonium bromide, benzalkonium bromide and/or cetylpyrdinium chloride.

85. The composition according to embodiment 82, wherein the zwitterionic emulsifier is phosphatidylcholine, betaine monohydrate, or Dehyton K®.

86. The composition according to embodiment 82, wherein the nonionic emulsifier is PEG-30 stearate, glycerol monostearate, glycerol monooleate, glycerol monoisostearate, partial glyceride medium chain, Tween®, polyoxyethylene sorbitan fatty acid esters, polyoxyethylene (20) sorbitan monostearate, sorbitan laurate, sorbitan monooleate, sorbitan monopalmitate, sorbitan tristearate, sorbitan sesquiloleate, saccharose monostearate, saccharose cocoate, cetomagrogol 1000, macrogol cetostearyl ether, macrogol oleyl ether, lauromacrogol 400, cholesterol, wool fat, acetylated wool fat, hydrated wool fat, wool fat alcohols, macrogol 1000 glycerol monooleate, macrogol 1000 glycerol monostearate, macrogol 300 glycerol tris hydroxystearate, macrogol 5 glycerol stearate, macrogol glycerol hydroxystearate, triglycerol diisostearate

87. The composition according to embodiment 79, wherein the gelling agent is synthetic polyacrylic acid (carbomer), a semi-synthetic cellulose, xanthan, inorganic bentonite, and/or hypromellose.

88. The composition according to embodiment 79, wherein the preservative is an alkyl-4-hydroxybenzoate, sorbic acid, benzoic acid, benzyl alcohol, phenoxyethanol, phenyl ethyl alcohol, a high concentration of ethanol, a high concentration of 2-propanol and/or a high concentration of propylene glycol.

89. The composition according to embodiment 79, wherein the antioxidant is alpha-tocopherol, ethyl gallate, propyl gallate, octyl gallate, lauryl gallate, decyl ester gallate, butylhydroxyanisol, butylated hydroxytoluene and/or an ascorbic acid ester.

90. The composition according to any one of embodiments 75-78, wherein the composition comprises a penetration enhancer, a buffering agent, a tonicity modifier, and a surfactant.

91. The composition according to embodiment 90, wherein the composition comprises dimethyl sulfoxide at at least 5%, at least 10%, at least 15%, at least 20%, at least 25%, at least 30%, at least 35%, at least 40%, at least 45%, at least 50%, at least 55%, or at least 60%, 10 mM sodium phosphate; 0.8% w/v NaCl; and Polysorbate 20 at 0.01, 0.05, 0.1, 0.2, 0.4, 0.6, 0.8, 1, 2, 3, or 4 w/v %, wherein the composition has a pH of 5.0, 5.5, 6.0, 6.5, 7, 7.5, or 8.

92. The composition according to embodiment 91, wherein the composition comprises Polysorbate 20 at 0.05 w/v %, and wherein the composition has a pH of 6.0.

93. The composition according to claim 90, wherein the composition comprises dimethyl sulfoxide at at least 5%, at least 10%, at least 15%, at least 20%, at least 25%, at least 30%, at least 35%, at least 40%, at least 45%, at least 50%, at least 55%, or at least 60%, 10 mM sodium phosphate; 0.8% w/v NaCl; and Polysorbate 80 at 0.01, 0.05, 0.1, 0.2, 0.4, 0.6, 0.8, 1, 2, 3, or 4 w/v %, wherein the composition has a pH of 5.0, 5.5, 6.0, 6.5, 7, 7.5, or 8.

94. The composition according to embodiment 93, wherein the composition comprises Polysorbate 80 at 0.05 w/v %, and wherein the composition has a pH of 6.0.

95. The composition according to any one of claims 75-94, wherein topical application of a therapeutically effective amount of the peptide having at least 80% sequence identity to any of SEQ ID NOs: 1-260 decreases inflammation of the skin or mucosa including erythema, induration, scaling, itching, blistering, ulcerating, dryness, bleeding, peeling and/or necrotization.

### EXAMPLES

The Examples below describe the optimization of the methods disclosed herein. These Examples are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Example 1. Effect of Topically Administered Kv1.3 Channel Blocking Peptides on the Delayed Type Hypersensitivity Reaction in Rat

This example describes the evaluation of delayed-type hypersensitivity response after topical administration of a peptide of the disclosure, such as ShK-186 (SEQ ID NO:217) or ShK-198 (SEQ ID NO:210).

Model Description: The delayed type-hypersensitivity (DTH) model has been commonly used for years in drug discovery screening in relation to psoriasis and atopic dermatitis. This model is simple, reproducible and particularly useful for evaluation of potent anti-inflammatory therapeutics. It's performed by sensitizing rats on the abdomen with a hapten, in this case oxazolone, and a week later, challenging the pinna of the ear with the same sensitizing agent. This challenge causes an acute and robust DTH response which can be measured in terms of ear thickness and histopathology.

Materials: Female Lewis rats aged 9 weeks were procured from Charles River Labs (Stock #004). The hapten Oxazolone (4-ethoxymethylene-2-phenyl-oxazolin-5-one) and olive oil were purchased from Sigma; Acetone was purchased from EMD Millipore; DMSO (dimethyl sulfoxide) was purchased from Calbiochem; ShK-186 peptide was synthesized by CS Bio/Integrity Bio; ShK-198 peptide was synthesized by Peptides International; vehicle (designated P6N, 10 nM sodium phosphate, 0.8% sodium chloride, 0.05% polysorbate 20, Water for injection, pH 6) was formulated in-house. All P6N excipients were purchased from Sigma.

Topical Solutions: To make 5%, 1% or 0.2% ShK topical solutions, one hour before application, 150, 30 or 6 milligrams (based on lot peptide content) of ShK peptide were added to vehicle formulation of 2.7 milliliters of P6N and 0.3 milliliters DMSO for a final peptide concentration of 50 mg/mL (5% w/v), 10 mg/mL (1% w/v) or 2 mg/mL (0.2% w/v) respectively. The solutions were then mixed thoroughly. The negative control topical solution was made by adding 0.3 milliliters of DMSO to 2.7 milliliters of P6N vehicle to make a final concentration of 10% DMSO. This solution was then mixed by vortex. The final composition of the vehicle following all dilutions was 9 mM sodium phosphate, 0.72% NaCl, 0.045% polysorbate 20 and 10% DMSO. All topical solutions were made once and stored at 4° C. until the time of application. The solutions were used for the entire topical application regimen (48 hours).

Oxazolone Application: All animals were lightly anesthetized using an Isoflurane vaporizer (3%, 1 L/min 02) and the abdomens shaved to expose approximately 5 cm² of skin. For experimental sensitization, 100 microliters of freshly prepared 1% solution of oxazolone dissolved in a solution of 4:1 acetone to olive oil were applied to the shaved site. To obtain a delayed hypersensitivity response; six days after the first sensitizing application, 25 microliters of a freshly made 0.2% oxazolone challenge solution (in 4:1 acetone to olive oil) were applied to the outer aspects of each animal's right pinna. A null solution of 4:1 acetone/olive oil alone was applied in the same manner to each animal's left pinna. Baseline ear thicknesses were measured prior to the challenge using a dial engineer's caliper (SPI, model 24-360-0).

Peptide Treatments: At the time of application, 10 microliters of the 10 mg/mL topical solution (100 μg of peptide) were applied using a microliter pipet (Rainin Pipetman, P-20) in a spreading motion to the inside of the right pinna. Another 10 microliters of the 10 mg/mL (100 μg of peptide) were applied to outside of the right pinna. The total amount of ShK peptide applied to treatment animals per dose was 200 μg. The negative control topical solution was applied in the same manner to the right pinnae of animals in the negative control group.

Clinical Scores: Animals were lightly anesthetized using isoflurane and positioned to visualize pinnae on a lateral plane. Baseline, 24 hour and 48 hour measurements were taken from the same relative area of each ear. Using a dial caliper, the plunger was allowed to settle gently on the pinna in the general area of treatment. Five measurements for each ear at each time point were recorded.

Histology: 8 mm punch biopsies were harvested from the animal's right pinna, hemisectioned and fixed in 10% neutral buffered formalin. All samples were processed by HistoTox Inc. (Boulder, Colo.). Ear sections were paraffin-embedded and stained using an anti-CD8 antibody as well as hematoxylin-eosin (H&E). Slides were then scored at Bolder Biopath Inc. (Boulder, Colo.) by an independent pathologist for ear width, dermal/epidermal inflammation and CD8⁺ cell counts.

General Procedure: Female Lewis rats were caged in 3 treatment groups of 8 animals each. On the day before the application of the sensitizing hapten, all animals were pre-shaved to expose 5 cm² of skin so as not have any irritation from the procedure interfere with the sensitization. The following day (Day 0), the animals were sensitized with a 1% oxazolone solution. The day before challenge, the animals were lightly anesthetized and baseline ear measurements were recorded. On study day 6, the animals were treated with a ShK peptide or vehicle alone on their right ear at 3 hours and 1 hour pre-challenge. After application, the animals were challenged on their right ear with 0.2% oxazolone solution and acetone/olive oil was administered to the left ear to control for any swelling associated with the challenge vehicle. At 1 hour and 3 hours post-challenge, application was again carried out with either ShK or vehicle based on group allocation. On day 7, the animals were dosed 2 times in the morning and then lightly anesthetized for the 24 hour (post-challenge) time point. 24 hour measurements were recorded, and animals were dosed twice (2 hours apart) based on their respective treatment group. On the last day of the study (Day 8), the animals were dosed twice in the morning before the 48 hour post-challenge time point. Measurements were recorded and the animals then sacrificed via exsanguination and tissues harvested.

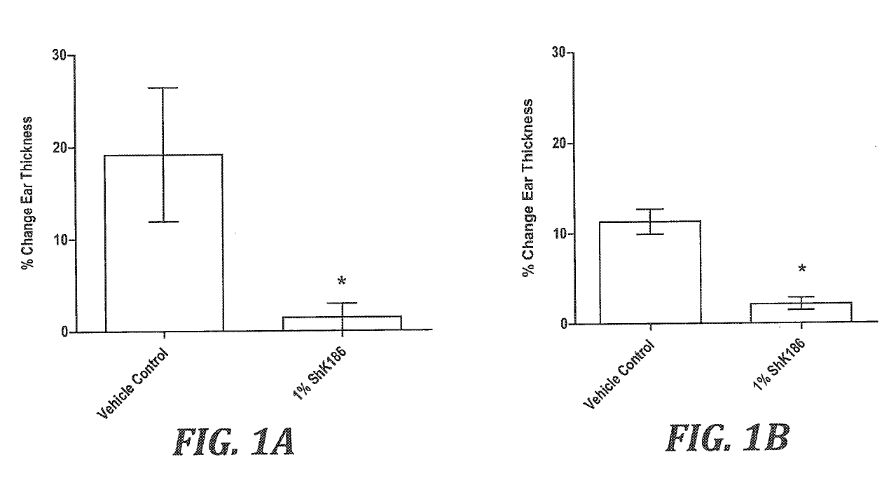

Using exemplary methods described above, results were obtained as shown in the following data. Average clinical scores of animals from 2 separate delayed-type hypersensitivity (DTH) reaction studies (FIG. 1A, FIG. 1B) at 24 hours post-challenge that were treated with either 1% ShK-186 or vehicle (9 mM sodium phosphate, 0.72% NaCl, 0.045% polysorbate 20, 10% DMSO) alone. Animals were topically administered 6 doses. *(P<0.05; t-test)

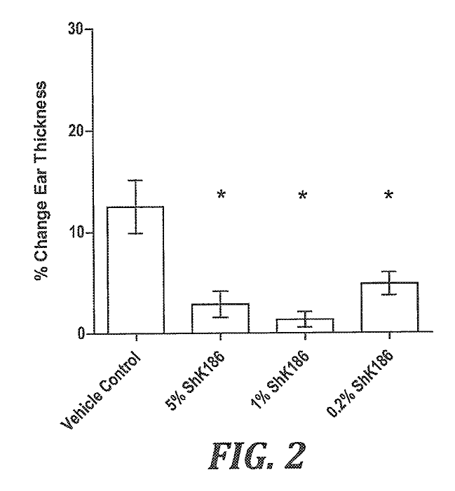

FIG. 2 shows average clinical scores of animals 24 hours post-challenge in a DTH hypersensitivity reaction study that were treated with either a 5%, 1% or 0.2% solution of ShK-186 or vehicle (9 mM sodium phosphate, 0.72% NaCl, 0.045% polysorbate 20, 10% DMSO) alone. Animals were topically administered 6 doses. *(P<0.05; ANOVA)

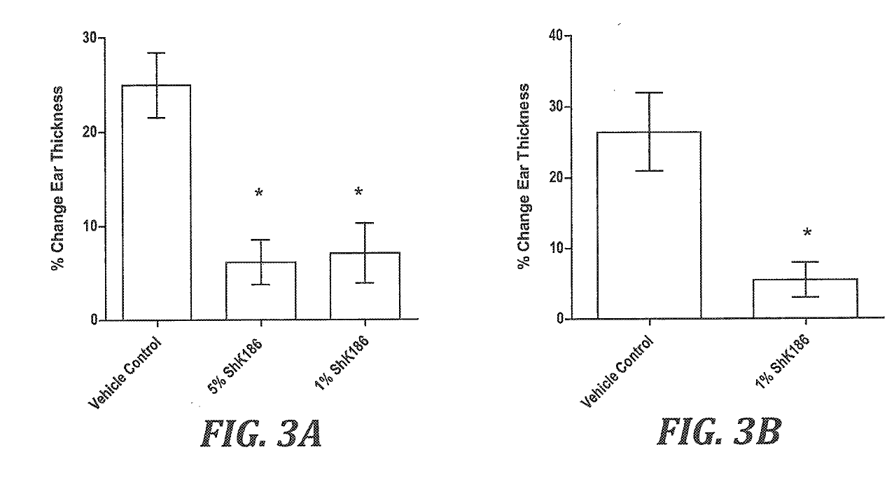

FIG. 3A shows average clinical scores of animals 48 hours post-challenge in a DTH hypersensitivity reaction study that were treated with either 5% or 1% solution of ShK-186 or vehicle (9 mM sodium phosphate, 0.72% NaCl, 0.045% polysorbate 20, 10%/DMSO) alone. Animals were topically administered 10 doses. *(P<0.05; ANOVA) FIG. 3B shows average clinical scores of animals from 48 hours post-challenge that were treated with a 1% solution of ShK-186 or vehicle alone. Animals were topically administered 10 doses *(P<0.05; t-test)

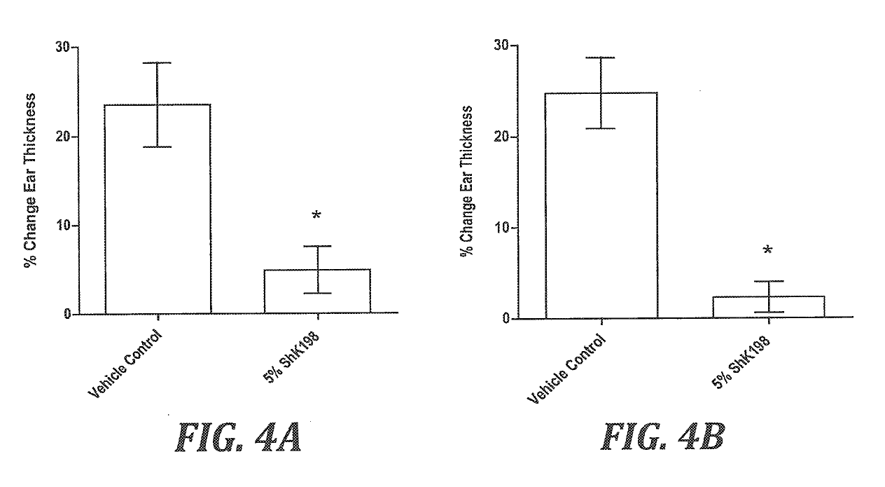

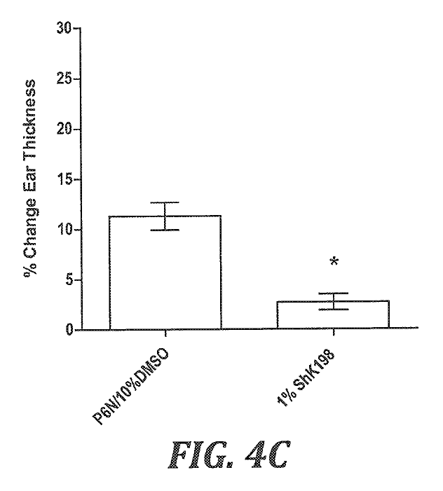

The average clinical scores of animals from 3 separate DTH hypersensitivity reaction studies (FIG. 4A, FIG. 4B, FIG. 4C) at 24 hours post-challenge that were treated with either a 5% or 1% solution of ShK-198 or vehicle (9 mM sodium phosphate, 0.72% NaCl, 0.045% polysorbate 20, 10% DMSO) alone. Animals were topically administered 6 doses. *(P<0.05; t-test)

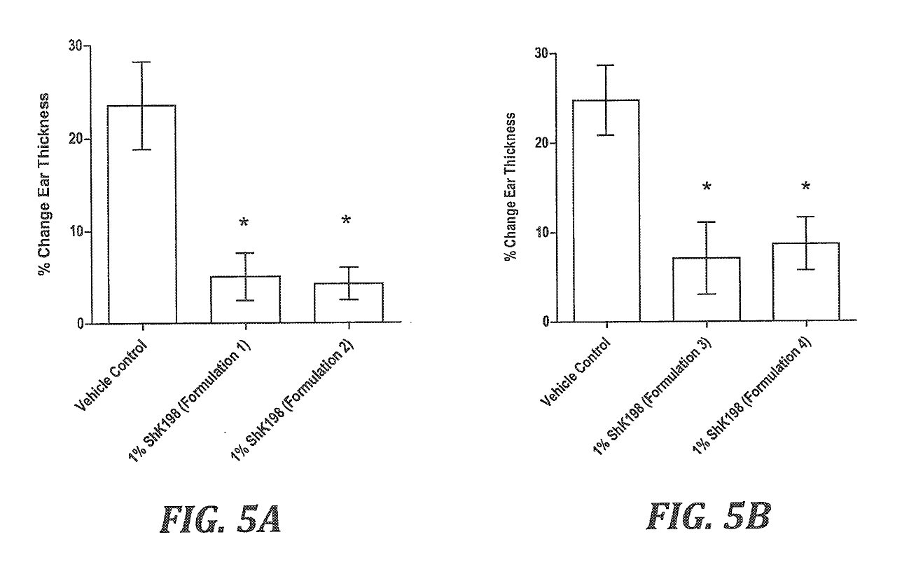

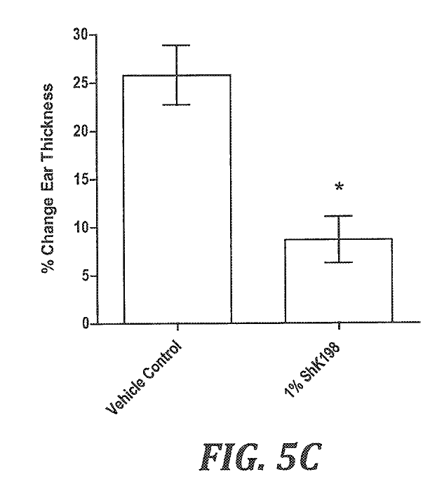

Average clinical scores of animals from 2 separate DTH hypersensitivity studies (FIG. 5A, FIG. 5B) 24 hours post-challenge that were treated with a 1% or solution of ShK-198 in multiple novel formulations or vehicle alone. Animals were topically administered 6 doses. *(P<0.05; ANOVA) FIG. 5C shows average clinical scores of animals from 48 hours post-challenge that were treated with a 1% or solution of ShK-198 or vehicle alone. Animals were topically administered 10 doses. *(P<0.05; t-test)

### Example 2. Topical Formulations for the Localized Delivery of the Disclosed Peptides

This example describes the formulation of the peptides of the disclosure, such as ShK-186 (SEQ ID NO:217) or ShK-198 (SEQ ID NO:210) for topical delivery to the skin.

Materials: ShK-186 toxin-based therapeutic peptide was synthesized by CS Bio/Integrity Bio; ShK-198 toxin-based therapeutic peptide was synthesized by Peptides International. Absolute ethanol was purchased from Fisher Scientific; DMI (Dimethyl Isosorbide) and propylene glycol were purchased from Croda Inc.; Acetone was purchased from EMD Millipore; DMSO (dimethyl sulfoxide) was purchased from Calbiochem; Transcutol (Diethylene glycol monoethyl ether) was purchased from Sigma. P6N vehicle (10 nM sodium phosphate, 0.8% sodium chloride, 0.05% polysorbate 20, Water for injection, pH 6) was formulated in-house. All P6N excipients were purchased from Sigma. Sterile, DNAse and RNAse-free polypropylene microcentrifuge tubes were purchased from Olympus Plastics.

Formulation 1: For 1 mL of a 1% solution, one hour before application: in a 1.8 mL polypropylene microcentrifuge tube, 10 milligrams (based on lot peptide content) of ShK peptide were added to a novel vehicle formulation of 0.45 milliliters of acetone, 0.36 milliliters absolute ethanol, 0.09 milliliters of DMSO and 0.10 milliliters of P6N for a final peptide concentration of 10 mg/mL. The topical composition was then mixed thoroughly for 15 seconds.

Formulation 2: For 1 mL of a 1% solution, one hour before application: in a 1.8 mL polypropylene microcentrifuge tube, 10 milligrams (based on lot peptide content) of ShK peptide were added to a novel vehicle formulation of 0.8 milliliters of DMI and 0.20 milliliters of P6N for a final peptide concentration of 10 mg/mL. The topical composition was then mixed thoroughly for 15 seconds.

Formulation 3: For 1 mL of a 1% solution, one hour before application: in a 1.8 mL polypropylene microcentrifuge tube, 10 milligrams (based on lot peptide content) of ShK peptide were added to a novel vehicle formulation of 0.7 milliliters of absolute ethanol, 0.10 milliliters of DMSO and 0.20 milliliters of P6N for a final peptide concentration of 10 mg/mL. The topical composition was then mixed thoroughly for 15 seconds.

Formulation 4: For 1 mL of a 1% solution, one hour before application: in a 1.8 mL polypropylene microcentrifuge tube, 10 milligrams (based on lot peptide content) of ShK peptide were added to a novel vehicle formulation of 0.25 milliliters of Transcutol, 0.25 milliliters of propylene glycol, 0.10 milliliters of P6N and 0.40 milliliters of P6N for a final peptide concentration of 10 mg/mL. The topical composition was then mixed thoroughly for 15 seconds.

### Example 3. Histology and Immunohistochemical Analysis of the DTH Response in Rats Treated with Topical Compositions of the Disclosure

This example describes the histopathological evaluation of delayed-type hypersensitivity response 48 hours post-challenge with a sensitizing agent after topical administration of a peptide of the disclosure, such as ShK-186 (SEQ ID NO:217) or ShK-198 (SEQ ID NO:210) in a topical composition.

Samples: All samples were from 9 week old Lewis rats that were used in a typical delayed-type hypersensitivity study. From these animals, 8 mm punch biopsies were harvested from the area of treatment and measurement on the right ear. The biopsies were then hemisectioned and fixed in 10% neutral buffered formalin. All samples were processed by HistoTox Inc. (Boulder, Colo.). Ear sections were paraffin-embedded and stained using an anti-CD8 antibody as well as hematoxylin-eosin (H&E). Slides were then scored at Bolder Biopath Inc. (Boulder, Colo.) by an independent board-certified veterinary pathologist for ear width, dermal/epidermal inflammation and CD8+ cell counts.

Morphologic pathology methods: H&E sections were prepared by standard methods. For the immunostaining, formalin-fixed, paraffin-embedded tissues were pretreated with proteinase K (CD8). After enzyme and protein blocking steps, tissues were incubated with primary antibodies (eBioscience, 14-0084 (CD8)) at room temperature. All slides were incubated with a secondary antibody to mouse IgG followed by incubation with Envision+Rabbit goat anti-Rabbit IgG HRP polymer (Dako, K4003). The stain was then developed using DAB+ solution (Dako, K3468). Slides are counterstained with Mayer's Hematoxylin, dehydrated, and permanently cover-slipped.

Ear Width: The ear thickness was measured at 4 sites starting at the base and progressing toward the tip with two equally spaced measurements (1000 μm intervals) taken proximal to the midpoint (toward head), one taken distal to the midpoint and one at the midpoint. Measure #1 is the midpoint, #2 is 1st proximal, #3 is 2nd proximal and #4 is distal (toward tip). This parameter documents edematous thickening and greater widths generally correlate with greater inflammation.

Microscopic Evaluation of Slides:

TABLE 4
Dermal Inflammation Scoring System

| | | |
|---|---|---|
| 0 | Normal | No inflammation |
| 1 | Minimal | Diffuse minor infiltrate, 1-2 inflammatory |
| | | cells/25X25 square μm area |
| 2 | Mild | Diffuse mild infiltrate, 3-5 inflammatory |
| | | cells/25X25 square μm area |
| 3 | Moderate | Diffuse moderate infiltrate of inflammatory cells, 6-8 |
| | | inflammatory cells/25X25 square μm area |
| 4 | Marked | Diffuse marked infiltrate of inflammatory cells, 9-12 |
| | | inflammatory cells/25X25 square μm area |
| 5 | Severe | Diffuse severe infiltrate of inflammatory cells, >12 |
| | | inflammatory cells/25X25 square μm area |

TABLE 5
Epidermal Inflammation Scoring System

| | | |
|---|---|---|
| 0 | Normal | No inflammation |
| 1 | Minimal | 1-2 epidermal accumulations of inflammatory cells or |
| | | scattered single cell accumulations, minor focal |
| | | or multifocal epidermal hyperplasia |
| 2 | Mild | 3-4 epidermal accumulations of inflammatory cells, mild |
| | | diffuse hyperplasia (skin thickness is 20-30 μm) |
| 3 | Moderate | 5-6 epidermal accumulations of inflammatory cells, |
| | | moderate diffuse hyperplasia (skin thickness is 30-50 μm) |
| 4 | Marked | 7-8 epidermal accumulations of inflammatory cells, |
| | | marked diffuse hyperplasia (skin thickness is 50-70 μm) |
| 5 | Severe | >8 epidermal accumulations of inflammatory cells, |
| | | severe diffuse hyperplasia (skin thickness is >70 μm) |

Summed Inflammation Score: The dermal and epidermal inflammation scores were summed.

CD8 Counts: Cells that stain positively for CD8 are counted at 400× in four 25×25 μm fields in the same locations as the ear width measurements. A mean of the four counts are determined for each animal.

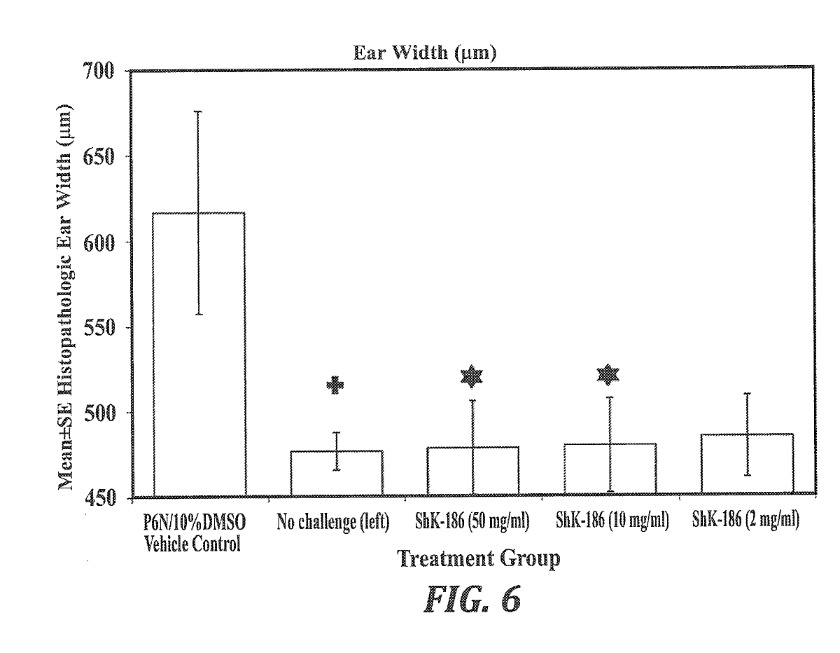

Using exemplary methods described above, results were obtained as shown in the following data. FIG. 6 shows average histopathologic rat ear widths (n=5/group) from animals used in a typical delayed-type hypersensitivity study. This graph represents animals that were treated with 5% (50 mg/mL), 1% (10 mg/mL) or 0.2% (2 mg/mL) ShK-186 peptide topical compositions. Vehicle control animals received P6N/10% DMSO alone. One group of animals were included that were sensitized, but not challenged as control for the model. (*P≤0.05 ANOVA to vehicle control; +P≤0.05 student's t-test to vehicle control (no challenge only)).

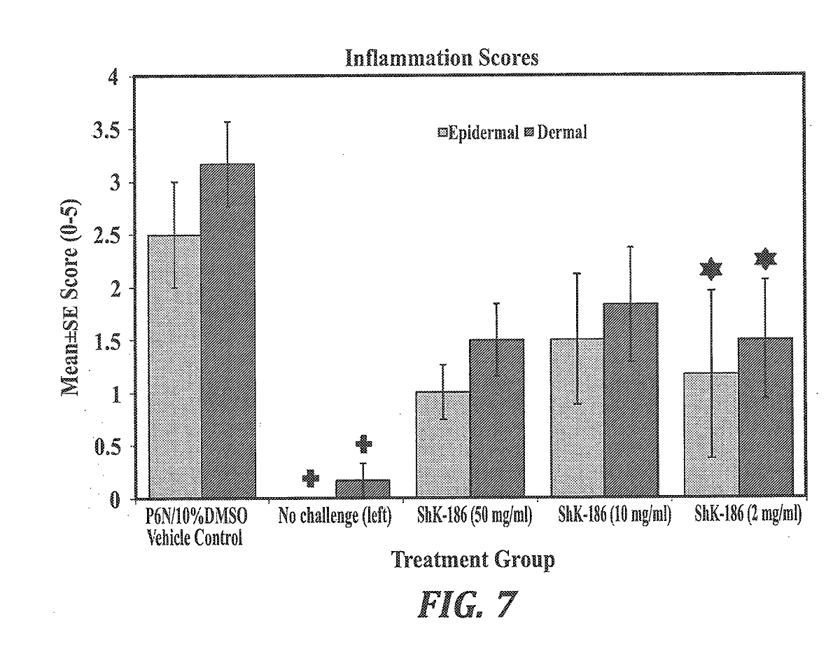

FIG. 7 shows average inflammation scores from animals enrolled in a typical delayed-type hypersensitivity study (n=5/group). Scoring was based on epidermal and dermal scoring scales as described. This graph represents animals that were treated with 5% (50 mg/mL), 1% (10 mg/mL) or 0.2% (2 mg/mL) ShK-186 peptide topical compositions. Vehicle control animals received P6N/10%/a DMSO alone. One group of animals included where the animals were sensitized, but not challenged as a control for the model. (*P≤0.05 ANOVA to vehicle control; +P≤0.05 student's t-test to vehicle control (no challenge only)).

FIG. 8 shows summed histopathology scores from animals used in a typical delayed-type hypersensitivity study (n=5/group). Percent differences from average vehicle control group scores are shown for each group. Scoring was based on the scoring scale provided above. This graph represents animals that were treated with 5% (50 mg/mL), 1% (10 mg/mL) or 0.2% (2 mg/mL) ShK-186 peptide topical solutions. Vehicle control animals received P6N/10% DMSO alone. One group of animals was included where the animals were sensitized, but not challenged as a control for the model. (*P≤0.05 ANOVA to vehicle control; +P≤0.05 student's t-test to vehicle control (no challenge only)).

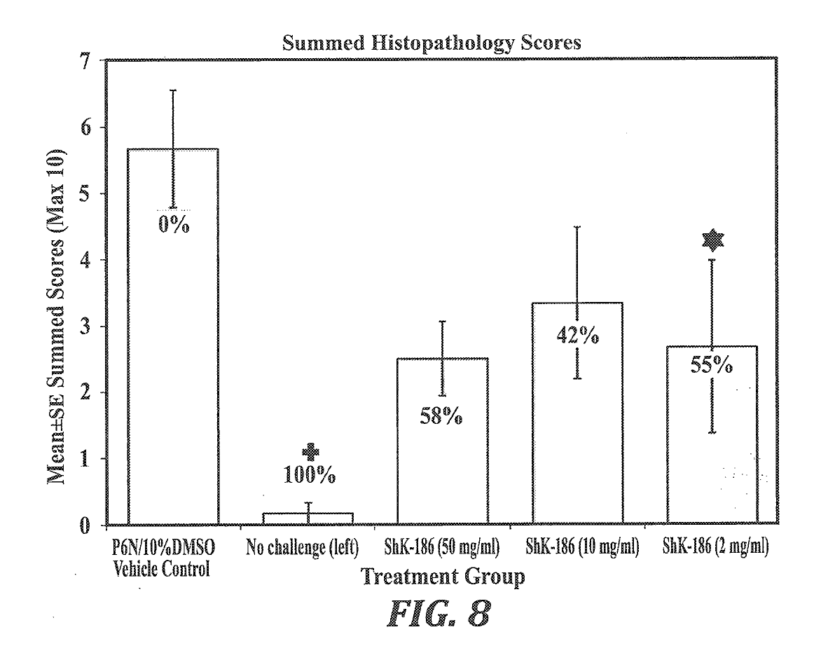

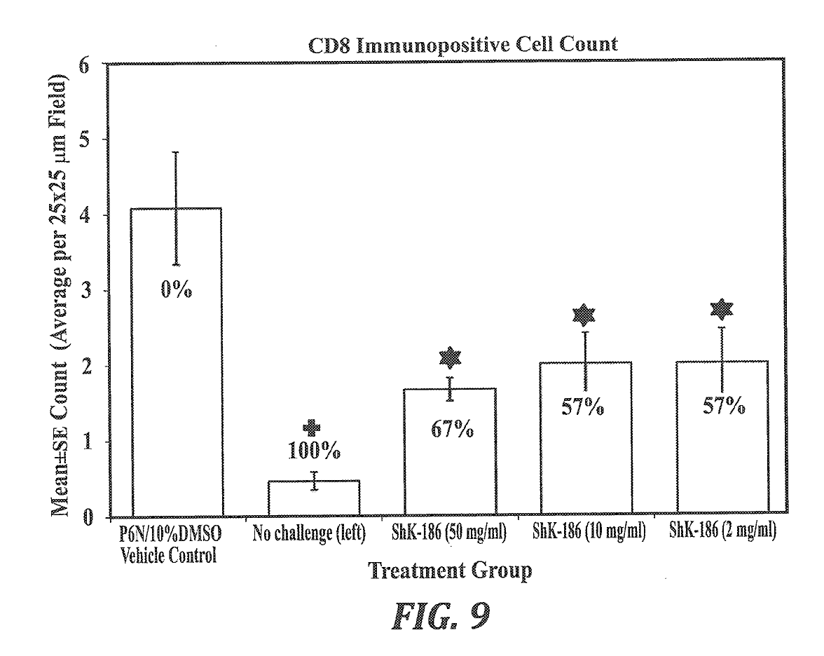

FIG. 9 shows CD8 positive cell counts from animals used in a typical delayed-type hypersensitivity study (n=5/group). Percent differences from average vehicle control group scores are shown for each group. Scoring was carried out as described. This graph represents animals that were treated with 5% (50 mg/mL), 1% (10 mg/mL) or 0.2% (2 mg/mL) ShK-186 peptide topical compositions. Vehicle control animals received P6N/10% DMSO alone. One group of animals were included that were sensitized, but not challenged as control for the model. (*P≤0.05 ANOVA to vehicle control; +P≤0.05 student's t-test to vehicle control (no challenge only)).

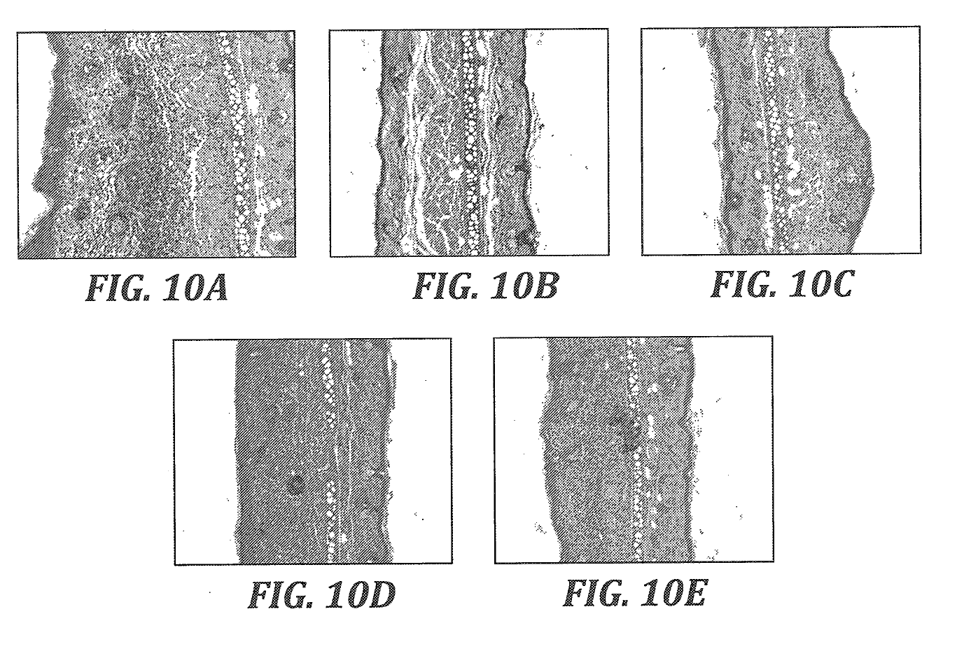

Photomicrographs of representative ears of each treatment group from animals in a typical DTH study: (FIG. 10A) Vehicle control group pinna (H&E, 100×) showing severe swelling and inflammation. (FIG. 10B) No challenge group pinna (H&E, 100×); normal appearance with no indications of inflammation. (FIG. 10C) 5% ShK-186 treatment group pinna (H&E, 100×) (FIG. 10D) 1% ShK-186 treatment group pinna (H&E, 100×). (FIG. 10E) 0.2% ShK-186 treatment group pinna (H&E, 100×).

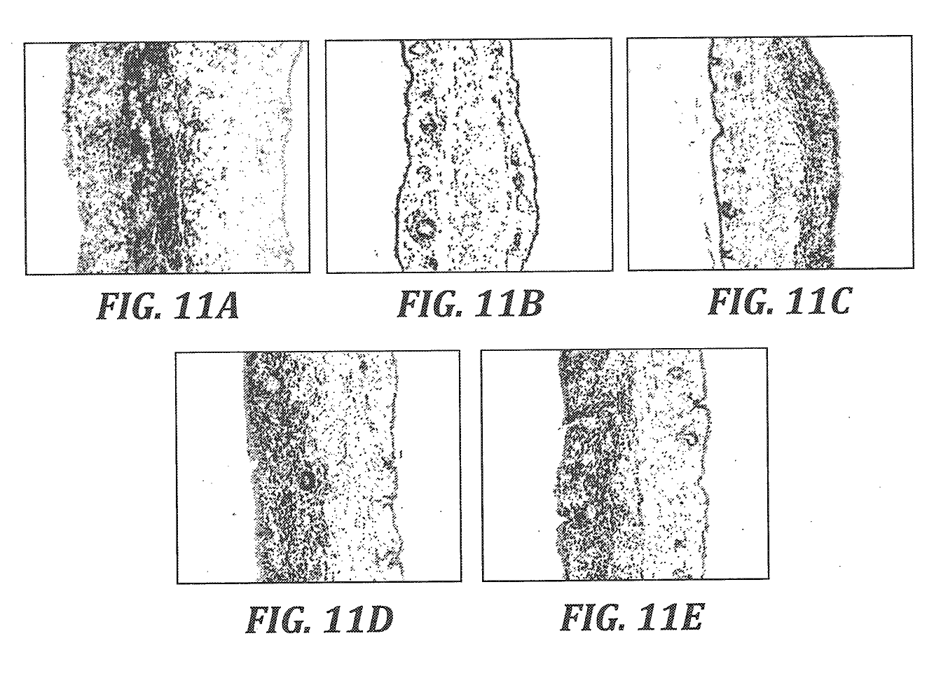

Photomicrographs detailing the dark staining of CD8 positive lymphocytes in representative ears of each treatment group from animals in a typical DTH study: (FIG. 11A) Vehicle control group pinna (CD8, 100×) showing severe CD8+ cell infiltration (dark stain). (FIG. 11B) No challenge group pinna (CD8, 100×); normal appearance with no indications of CD8+ cell infiltration. (FIG. 11C) 5% ShK-186 treatment group pinna (CD8, 100×) (FIG. 11D) 1% ShK-186 treatment group pinna (CD8, 100×). (FIG. 11E) 0.2% ShK-186 treatment group pinna (CD8, 100×).

### Example 4. Measuring Tissue Penetration of Topically Formulated Peptides of the Disclosure by Immunohistochemistry

This example describes the method of detection of toxin-based therapeutic peptides of the disclosure, such as ShK-186 (SEQ ID NO:217) or ShK-198 (SEQ ID NO:210) penetration after topical administration.

Materials: All Bond reagents such Bond HIER 1 (Citrate), Bond HIER 2 (EDTA), Bond Primary antibody diluent, Bond dewax solution, Bond Wash solution, Bond Polymer Refine (DAB) detection rack including peroxide block, polymer/hematoxylin, Bond Alcohol and the Leica Bond Automated Immunostainer were purchased from Leica-microsystems.

ShK specific antibody: Hybridoma fusions were performed using the isolated splenocytes from ShK-186 immunized mice and clones producing specific monoclonal antibodies against ShK-186 and ShK-198 were isolated. These cultures were subjected to a second cloning process using the limited dilution method and selected for stable expression of the specific mAb. Stable clones were expanded, tested for antibody specificity to ShK-186 (and ShK parent) by ELISA, and their isotypes were identified using the Roche Isostrips and confirmed by ELISA. For ELISA testing, culture supernatants were diluted from 1:4-1:256 and the titer was determined to be the last dilution 2 standard deviations above background. Clone P3B3-G11-C12 is purified from hybridoma extinction cultures and concentrated to 1 mg/ml. All clones were archived in liquid nitrogen at Kineta and Fred Hutchinson Cancer Research Center Monoclonal Development Laboratory.

Samples: All samples were harvested from rats. From these animals, 8 mm punch biopsies were harvested from the area of treatment or topical application on the ear. The biopsies were then hemisectioned and fixed in 10%/o neutral buffered formalin. Ear sections were paraffin-embedded and stained using an ShK specific monoclonal antibody.

General staining procedure: Slides were baked for 30 minutes at 60° C. and deparaffinized on the Leica Bond Automated Immunostainer. For antigen retrieval, HIER 1 (Citrate) or HIER 2 (EDTA) were used for 10 or 20 min, at 100° C. Proteinase kinase antigen retrieval was carried out for 15 min. The samples were blocked with normal 10% goat serum for 20 minutes at room temperature. The primary anti-ShK antibody (P3B3-G11-C12) was diluted 1:500 in Leica Primary antibody diluent for 30 minutes at room temperature. Following the primary antibody staining, Leica Bond Peroxide was used as a blocking agent for 5 minutes at room temperature. Leica Bond Mixed Refine (DAB) detection reagent was applied for 10 minutes at room temperature. The slides were hematoxylin counterstained on the Leica Bond Automated Immunostainer and cleared to xylene.

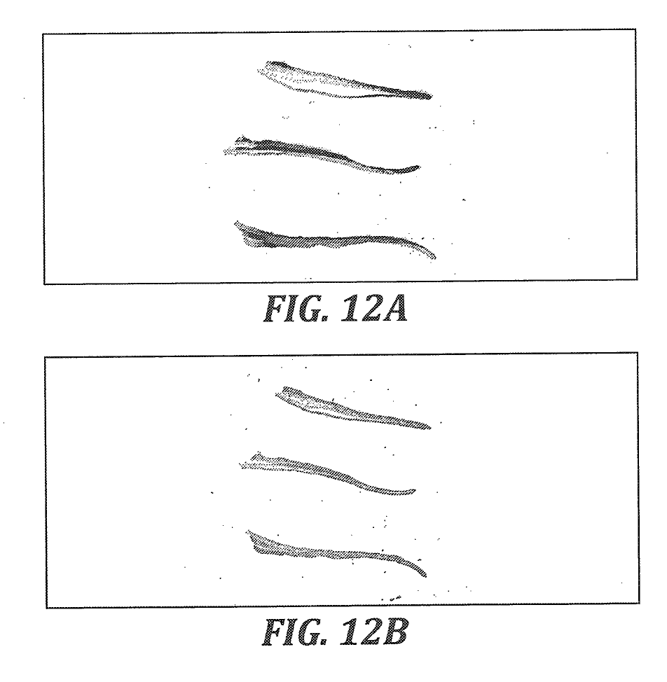

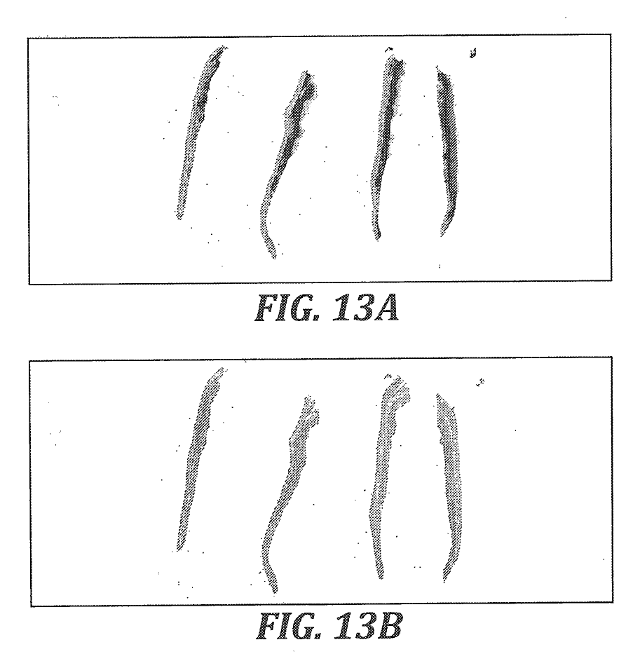

Using exemplary methods described above, results were obtained as shown in the following data. FIG. 12A shows rat pinna sections from an animal 48 hours post-challenge that was treated with a 1% solution of ShK-186 and stained with antibody from clone P3B3, a ShK specific monoclonal antibody. This animal was topically administered 10 doses. The dark staining shows perfuse and extensive penetration by the ShK-186 peptide. FIG. 12B shows rat pinna from same animal stained with isotype control. Lack of dark staining indicates the P3B3 antibody is specific the ShK-186 peptide.

FIG. 13A shows rat pinna sections from an animal 48 hours post-challenge that was treated with a 5% solution of ShK-198 and stained with clone P3B3, a ShK specific monoclonal antibody. This animal was topically administered 10 doses. The dark staining shows perfuse and extensive penetration by the ShK-198 peptide. FIG. 13B shows rat pinna from same animal stained with isotype control. Lack of dark staining indicates the P3B3 antibody is specific the ShK-198 peptide.

As will be understood by one of ordinary skill in the art, each embodiment disclosed herein can comprise, consist essentially of, or consist of its particular stated element, step, ingredient or component. Thus, the terms “include” or “including” should be interpreted to recite: “comprise, consist of, or consist essentially of.” The transitional term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would prevent the particular embodiment from achieving sustained release of a therapeutic protein as “sustained release” is defined herein.

Unless otherwise indicated, all numbers used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; +13% of the stated value; +12% of the stated value; +11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently includes certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to include the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventors expect skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to publications, patents and/or patent applications (collectively “references”) throughout this specification. Each of the cited references is individually incorporated herein by reference for their particular cited teachings.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

In closing, it is to be understood that the embodiments of the invention disclosed herein are illustrative of the principles of the present disclosure. Other modifications that may be employed are within the scope of the disclosed methods and compositions. Thus, by way of example, but not of limitation, alternative configurations of the present disclosure can be utilized in accordance with the teachings herein. Accordingly, the present disclosure is not limited to that precisely as shown and described.

The embodiments of the invention in which an exclusive property or privilege is claimed are defined as follows:

1. A method of treating inflammation of the skin or mucosa in a subject in need thereof comprising administering topically to the subject a therapeutically effective amount of a pharmaceutical composition including at least one ShK-based peptide having the amino acid sequence of any of SEQ ID NOs:1-224 and SEQ ID NOs:257-260 and a pharmaceutically acceptable carrier.

2. A method of treating inflammation of the skin or mucosa in a subject in need thereof including administering topically to the subject a therapeutically effective amount of a pharmaceutical composition comprising an ShK-based peptide having the formula SEQ ID NOs:208-224 and SEQ ID NOs:257-260 and a pharmaceutically acceptable carrier.

3. A method of claim 1 wherein the ShK-based peptide has the formula SEQ ID NO:210.

4. A method of claim 1 wherein the ShK-based peptide has the formula SEQ ID NO:217.

5. A method of treating inflammation of the skin or mucosa in a subject in need thereof comprising administering topically to the subject a therapeutically effective amount of a pharmaceutical composition comprising an ShK-based peptide having a sequence that has at least 95% sequence identity to at least one of SEQ ID NO:1, SEQ ID NO:2, SEQ ID NO:208, SEQ ID NO:209, SEQ ID NO:210, SEQ ID NO:217, and SEQ ID NO:218.

6. A method of claim 1 or 5, wherein the toxin-based therapeutic peptide is natural or synthetic.

7. A method of claim 1 or 5, wherein the toxin-based therapeutic peptide is attached to an organic or inorganic chemical entity that has an anionic charge.

8. A method of claim 1 or 5, wherein the C-terminus of the toxin-based therapeutic peptide is an amide.

9. A method of any one of claims 1-8, wherein the skin or mucosal inflammatory condition is or is caused by cutaneous systemic lupus erythematosis, dermatomyositis, scleroderma, psoriasis, atopic dermatitis, vasculitis, Bechet's syndrome, Henoch-Schonlein prupura, hypersensitivity reactions, Kawasaki disease, microscopic polyangitis, polyarteritis nodosa, vitiligo, alopecia areata, autoimmune progesterone dermatitis, Henoch-Schonlein purpura, Blau syndrome, bullous penphigoid, Churg-Strauss syndrome, cicatricial pemphigoid, contact dermatitis, Crohn's disease, inflammatory bowel disease, dermatitis herpetiformis, diffuse cutaneous systemic sclerosis, discoid lupus erythematosus, ecaema, eosinophilic fasciitis, epidermolysis bullosa acquisita, erythema nodosum, lichen planus, lupus erythematosus, Majeed syndrome, morphea, Mucha-Habermann disease, Parry-Romberg syndrome, POEMS syndrome, psoriasis, pyoderma gangrenosum, relapsing polychondritis, reactive arthritis, rheumatoid arthritis, scleroderma, Sjogren's syndrome, systemic lupus erythematosis, vasculitis and Wegener's granulomatosis, microscopic polyangiitis, polyarteritis nodosa, Takayasu's arteritis, rheumatoid arthritis, relapsing polychondritis or ankylosing spondylitis.

10. A method of any one of claims 1-9, wherein the pharmaceutical composition contains at least one dermatological base selected from: water, ethanol, 2-propanol, glycerol, propylene glycol, sorbitol, macrogol, dimethyl sulfoxide, acetone, petrolatum, hard paraffin, soft and liquid paraffin, triglycerides, wax, liquid wax ester, partial glycerides, silicon oils, anionic surfactants, zwitterionic surfactants, nonionic surfactants, bentonite, carbomer, carmellose sodium, hydroxyethyl cellulose, hydroxypropyl cellulose, hypromellose, alkyl-4-hydroxybenzoates, sorbic acid, benzyl alcohol, phenylethyl alcohol, butylhydroxyanisol, butylated hydroxytoluene, tocopherol, tocopherol acetate and ascorbyl palmitate propylene glycol, yellow wax, fluid wax esters, isopropyl myristate, ethylhexyl palmitate, dimethicone, phenyl methyl polysiloxane and/or cyclomethicone.

11. A method of claim 1 or 5, wherein the therapeutically effective amount decreases inflammation of the skin or mucosa including erythema, induration, scaling, itching, blistering, ulcerating, dryness, bleeding, peeling and/or necrotization.

12. A method of evaluating a subject to predict the outcome of treatment with a method of claim 1 or 5 including: analyzing Kv1.3 channel expression levels of T-cells from a biological sample of the subject; wherein increased levels of Kv1.3 channel expression relative to a healthy control or reference population is indicative of a patient receptive to treatment with an ShK-based peptide having at least 80% sequence identity to any one of SEQ ID NOs: 1-224 and SEQ ID NOs:257-260.

13. A method of determining if a subject will benefit from treatment with a method of claim 1 or 5 including: measuring Kv1.3 channel expression levels of at least one of T-cells and macrophages from a biological sample of the subject; comparing Kv1.3 channel expression levels of the cells to that of at least one of T-cells and macrophages from a healthy control or reference population; and determining that the subject will benefit from treatment with a method of claim 1 or 5 if the level of Kv1.3 channel expression in the subject is increased compared to the healthy control or reference population.

14. A composition for use in the topical treatment of inflammation of the skin or mucosa in a subject in need thereof comprising a therapeutically effective amount of at least one peptide having an amino acid sequence at least 95% identical to any of SEQ ID NOs:1-224 and SEQ ID NOs:257-260 and a dermatological base and/or a pharmaceutically acceptable carrier.

15. A composition for use in the topical treatment of inflammation of the skin or mucosa in a subject in need thereof comprising a therapeutically effective amount of a pharmaceutical composition comprising an peptide having an amino acid sequence at least 95% identical to any one of SEQ ID NOs:208-224 and SEQ ID NOs:257-260 and a dermatological base and/or a pharmaceutically acceptable carrier.

16. The composition according to claim 14, wherein the peptide has at least 95% identity to at least one of SEQ ID NO:1, SEQ ID NO:2, SEQ ID NO:208, SEQ ID NO:209, SEQ ID NO:210, SEQ ID NO:217, and SEQ ID NO:218.

17. A composition for use in the topical treatment of inflammation of the skin or mucosa inflammatory condition in a subject in need thereof comprising a therapeutically effective amount of a pharmaceutical composition comprising a peptide having an amino acid sequence at least 95% identical to any one of SEQ ID NOs: 1 to 224 or 257-260, wherein the inflammation of the skin or mucosa is or is caused by cutaneous lupus erythematosis, dermatomyositis, scleroderma, psoriasis, atopic dermatitis, vasculitis, Bechet's syndrome, Henoch-Schonlein prupura, hypersensitivity reactions, Kawasaki disease, microscopic polyangitis, polyarteritis nodosa, vitiligo, alopecia areata, autoimmune progesterone dermatitis, Henoch-Schonlein purpura, Blau syndrome, bullous penphigoid, Churg-Strauss syndrome, cicatricial pemphigoid, contact dermatitis, Crohn's disease, inflammatory bowel disease, dermatitis herpetiformis, diffuse cutaneous systemic sclerosis, discoid lupus erythematosus, ecaema, eosinophilic fasciitis, epidermolysis bullosa acquisita, erythema nodosum, lichen planus, lupus erythematosus, Majeed syndrome, morphea, Mucha-Habermann disease, Parry-Romberg syndrome, POEMS syndrome, psoriasis, pyoderma gangrenosum, relapsing polychondritis, reactive arthritis, rheumatoid arthritis, scleroderma, systemic lupus erythematosis, vasculitis and Wegener's granulomatosis, Takayasu's arteritis, or ankylosing spondylitis.

18. The composition according to any one of claims 14 to 17, wherein the dermatological base comprises a penetration enhancer, a hydrophilic emulsifying agent, a hydrophilic gelling agent, a hydrophobic emulsifying agent, a hydrophobic gelling agent, a preservative and/or an antioxidant.

19. The composition according to claim 18, wherein the dermatological base further comprises water, ethanol, 2-propanol, glycerol, propylene glycol, sorbitol, macrogol, dimethyl sulfoxide, acetone, petrolatum, hard paraffin, soft and liquid paraffin, triglycerides, wax, liquid wax ester, a partial glyceride, a silicon oil, an anionic surfactant, a zwitterionic surfactant, a nonionic surfactant, bentonite, carbomer, carmellose sodium, hydroxyethyl cellulose, hydroxypropyl cellulose, hypromellose, alkyl-4-hydroxybenzoates, sorbic acid, benzyl alcohol, phenylethyl alcohol, butylhydroxyanisol, butylated hydroxytoluene, tocopherol, tocopherol acetate, ascorbyl palmitate, and/or combinations of any one or more of the above.

20. The composition according to claim 19, wherein the hydrophobic base is a yellow wax, a fluid wax esters, or a silicone oil that contains polyorganoziloxane as the functional group.

21. The composition according to claim 18, wherein the emulsifier is nonionic, anionic, cationic and/or zwitterionic.

22. The composition according to claim 21, wherein the anionic emulsifier is sodium stearate, aluminum stearate, sodium dodecyl sulfate, sodium cetyl stearyl sulfate, sodium lauryl ether sulfate, and/or sodium dioctyl sulfosuccinate.

23. The composition according to claim 21, wherein the cationic emulsifier is cetyl trimethyl ammonium bromide, benzalkonium bromide and/or cetylpyrdinium chloride.

24. The composition according to claim 21, wherein the zwitterionic emulsifier is phosphatidylcholine, betaine monohydrate, or Dehyton K®.

25. The composition according to claim 21, wherein the nonionic emulsifier is PEG-30 stearate, glycerol monostearate, glycerol monooleate, glycerol monoisostearate, partial glyceride medium chain, Tween®, polyoxyethylene sorbitan fatty acid esters, polyoxyethylene (20) sorbitan monostearate, sorbitan laurate, sorbitan monooleate, sorbitan monopalmitate, sorbitan tristearate, sorbitan sesquiloleate, saccharose monostearate, saccharose cocoate, cetomagrogol 1000, macrogol cetostearyl ether, macrogol oleyl ether, lauromacrogol 400, cholesterol, wool fat, acetylated wool fat, hydrated wool fat, wool fat alcohols, macrogol 1000 glycerol monooleate, macrogol 1000 glycerol monostearate, macrogol 300 glycerol tris hydroxystearate, macrogol 5 glycerol stearate, macrogol glycerol hydroxystearate, triglycerol diisostearate

26. The composition according to claim 18, wherein the penetration enhancing agent is a sulfoxide, an azone, a pyrrolidone, a glycol, a surfactant, and/or a terpene.

27. The composition according to claim 18, wherein the gelling agent is synthetic polyacrylic acid (carbomer), a semi-synthetic cellulose, xanthan, inorganic bentonite, and/or hypromellose.

28. The composition according to claim 18, wherein the preservative is an alkyl-4-hydroxybenzoate, sorbic acid, benzoic acid, benzyl alcohol, phenoxyethanol, phenyl ethyl alcohol, a high concentration of ethanol, a high concentration of 2-propanol and/or a high concentration of propylene glycol.

29. The composition according to claim 18, wherein the antioxidant is alpha-tocopherol, ethyl gallate, propyl gallate, octyl gallate, lauryl gallate, decyl ester gallate, butylhydroxyanisol, butylated hydroxytoluene and/or an ascorbic acid ester.

30. The composition according to any one of claims 14-17, wherein the composition comprises a penetration enhancer, a buffering agent, a tonicity modifier, and a surfactant.

31. The composition according to claim 30, wherein the composition comprises dimethyl sulfoxide at at least 5%, at least 10%, at least 15%, at least 20%, at least 25%, at least 30%, at least 35%, at least 40%, at least 45%, at least 50%, at least 55%, or at least 60%, 10 mM sodium phosphate; 0.8% w/v NaCl; and Polysorbate 20 at 0.01, 0.05, 0.1, 0.2, 0.4, 0.6, 0.8, 1, 2, 3, or 4 w/v %, wherein the composition has a pH of 5.0, 5.5, 6.0, 6.5, 7, 7.5, or 8.

32. The composition according to claim 31, wherein the composition comprises Polysorbate 20 at 0.05 w/v %, and wherein the composition has a pH of 6.0.

33. The composition according to claim 30, wherein the composition comprises dimethyl sulfoxide at at least 5%, at least 10%, at least 15%, at least 20%, at least 25%, at least 30%, at least 35%, at least 40%, at least 45%, at least 50%, at least 55%, or at least 60%, 10 mM sodium phosphate; 0.8% w/v NaCl; and Polysorbate 80 at 0.01, 0.05, 0.1, 0.2, 0.4, 0.6, 0.8, 1, 2, 3, or 4 w/v %, wherein the composition has a pH of 5.0, 5.5, 6.0, 6.5, 7, 7.5, or 8.

34. The composition according to claim 33, wherein the composition comprises Polysorbate 80 at 0.05 w/v %, and wherein the composition has a pH of 6.0.

35. The composition according to any one of claims 14-34, wherein topical application of a therapeutically effective amount of the peptide having at least 80% sequence identity to any of SEQ ID NOs: 1-260 decreases inflammation of the skin or mucosa including erythema, induration, scaling, itching, blistering, ulcerating, dryness, bleeding, peeling and/or necrotization.
